(19) United States
(12) Patent Application Publication
(10) Pub. No.: US 2026/0349338 A1
(43) Pub. Date: Nov. 5, 2026

(54) RESIST COMPOSITION, RESIST PATTERN FORMATION METHOD, AND COMPOUND AND INTERMEDIATE THEREFOR

(71) Applicant: TOKYO OHKA KOGYO CO., LTD., Kawasaki-shi (JP)

(72) Inventors: Hiroki KATO, Kawasaki-shi (JP); Takuya UEHARA, Kawasaki-shi (JP)

(21) Appl. No.: 19/104,807

(22) PCT Filed: Aug. 10, 2023
(86) PCT No.: PCT/JP2023/029309

§ 371 Date: Feb. 19, 2025

(30) Foreign Application Priority Data
Aug. 22, 2022 (JP) 2022-132007

(51) Int. Cl. G03F 7/004; C07C 309/12; C07C 321/28; C07D 333/76; G03F 7/038; G03F 7/039; G03F 7/30

(52) U.S. Cl. CPC G03F 7/0046; C07C 309/12; C07C 321/28; C07D 333/76; G03F 7/0382; G03F 7/0388; G03F 7/0397; C07C 2603/88; G03F 7/30

(57) ABSTRACT

A resist composition including a base material component whose solubility in a developing solution is changed by an action of an acid and a compound represented by General Formula (b0). In Formula (b0), M^{m+} represents an m-valent organic cation. R⁰ represents a fluorinated alkyl group or a fluorine atom. Vb⁰ represents an alkylene group, a fluorinated alkylene group, or a single bond. Yb⁰ represents a divalent linking group. Rpg represents an acid dissociable group represented by General Formula (a0-pg). In Formula (a0-pg), A represents a cyclic alkene having only one carbon-carbon unsaturated bond in a ring skeleton. Ra⁰¹ represents a monovalent hydrocarbon group. I represents an iodine atom. m1 represents an integer of 0 to 20. m2 represents an integer of 1 to 20. * represents a bonding site

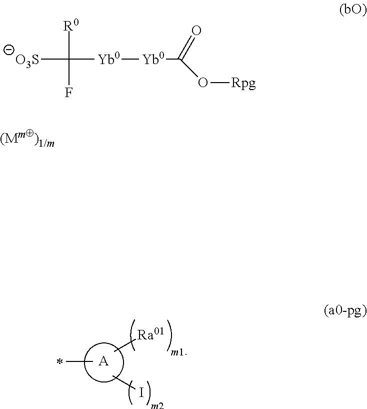

### TECHNICAL FIELD

The present invention relates to a resist composition, a resist pattern formation method, a compound, and an intermediate therefor.

Priority is claimed on Japanese Patent Application No. 2022-132007, filed Aug. 22, 2022, the content of which is incorporated herein by reference.

### BACKGROUND ART

In recent years, in the production of semiconductor elements and liquid crystal display elements, advances in lithography technologies have led to a rapid progress in the field of pattern fining. These pattern miniaturization techniques typically involve shortening the wavelength (increasing the energy) of the exposure light source.

Resist materials are required to have lithography characteristics such as sensitivity to these exposure light sources and resolution that enables reproduction of patterns with minute dimensions.

As a resist material that satisfies these requirements, a chemically amplified resist composition containing a base material component whose solubility in a developing solution is changed by an action of an acid and an acid generator component that generates an acid upon light exposure has been used.

In the resist pattern formation, the behavior of an acid generated from an acid generator component upon light exposure is considered as one factor that has a great influence on lithography characteristics.

As the acid generator that is used in the chemically amplified resist composition, a wide variety of acid generators been suggested in the related art. Examples of such known acid generators include an onium salt-based acid generator such as an iodonium salt or a sulfonium salt, an oxime sulfonate-based acid generator, a diazomethane-based acid generator, a nitrobenzyl sulfonate-based acid generator, an imino sulfonate-based acid generator, and a disulfone-based acid generator.

An alkyl sulfonate ion or a fluorinated alkyl sulfonate ion in which some or all hydrogen atoms of the alkyl group have been substituted with fluorine atoms is typically used in an anion moiety of the onium salt-based acid generator. As a cation moiety of the onium salt-based acid generator, a cation moiety including an onium ion such as triphenylsulfonium is mainly used.

For example, Patent Document 1 discloses a resist composition containing a sulfonium-based acid generator containing an electron-withdrawing group in a cation moiety. According to this resist composition, the sensitivity can be increased.

### CITATION LIST

### Patent Document

Patent Document 1: Japanese Unexamined Patent Application, First Publication No. 2017-227734

### SUMMARY OF INVENTION

### Technical Problem

With further advances in lithography technologies, rapid progress in the field of pattern fining is being achieved together with the expansion of application fields. In association with this, in a case of manufacturing a semiconductor element or the like, there is a demand for a technology that enables formation of a fine pattern in a satisfactory shape. For example, in lithography using extreme ultraviolet (EUV) rays or electron beams (EB), a target is to form a fine pattern with a size of several tens of am. As the pattern dimensions decrease, it is required to improve lithography characteristics such as sensitivity and resolution without trade-off. Further, with the resist pattern fining, a problem of development loss (film reduction) may occur due to excessive dissolution of particularly unexposed portions of the resist film in a developing solution during development.

The present invention has been made in consideration of the above-described circumstances, and an object thereof is to provide a resist composition capable of achieving high sensitivity in a case of formation of a resist pattern and suppressing a decrease in film thickness during development, a resist pattern formation method using the resist composition, and a compound and an intermediate thereof, which are useful as an acid generator component used in the resist composition.

### Solution to Problem

In order to solve the above-described problems, the present invention has adopted the following configurations.

That is, according to a first aspect of the present invention, there is provided a resist composition which generates an acid upon light exposure and whose solubility in a developing solution is changed by an action of the acid, the resist composition including: a base material component (A) whose solubility in a developing solution is changed by the action of the acid; and a compound (B0) represented by General Formula (b0).

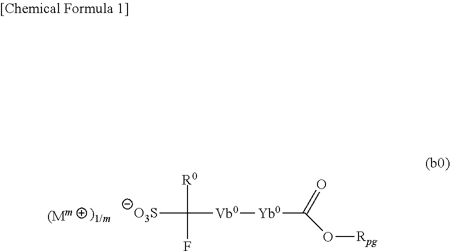

[In Formula (b0), M^{m+} represents an m-valent organic cation. m represents an integer of 1 or greater. R⁰ represents a fluorinated alkyl group having 1 to 5 carbon atoms or a fluorine atom. Vb⁰ represents an alkylene group, a fluorinated alkylene group, or a single bond. Yb⁰ represents a divalent linking group. Rpg represents an acid dissociable group represented by General Formula (a0-pg).]

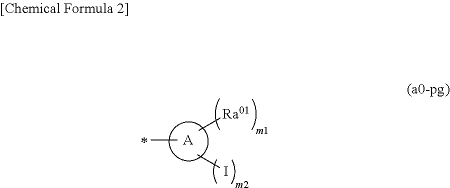

[In Formula (a0-pg), A represents a cyclic alkene having only one carbon-carbon unsaturated bond in a ring skeleton. Ra⁰¹ represents a monovalent hydrocarbon group having 1 to 20 carbon atoms. I represents an iodine atom. m1 represents an integer of 0 to 20. m2 represents an integer of 1 to 20. Here, m1+m2≤(a total number of carbon atoms constituting the ring skeleton of the cyclic alkene as A-1) is satisfied. In a case where m1 represents an integer of 2 or greater, a plurality of Ra⁰¹'s may be the same as or different from each other. * represents a bonding site that is bonded to —O— (oxy group) in —Yb⁰—CO—O—.]

According to a second aspect of the present invention, there is provided a resist pattern formation method including: a step of forming a resist film on a support using the resist composition according to the first aspect; a step of exposing the resist film to light, and a step of developing the resist film exposed to light to form a resist pattern.

According to a third aspect of the present invention, there is provided a compound represented by General Formula (b0),

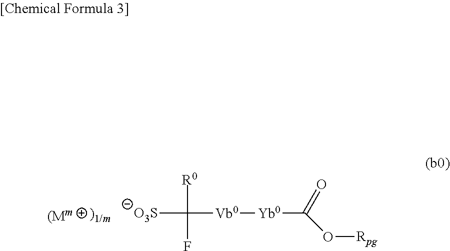

[In Formula (b0), M^{m+} represents an m-valent organic cation, m represents an integer of 1 or greater. R⁰ represents a fluorinated alkyl group having 1 to $ carbon atoms or a fluorine atom. Vb⁰ represents an alkylene group, a fluorinated alkylene group, or a single bond. Yb⁰ represents a divalent linking group. Rpg represents an acid dissociable group represented by General Formula (a0-pg).]

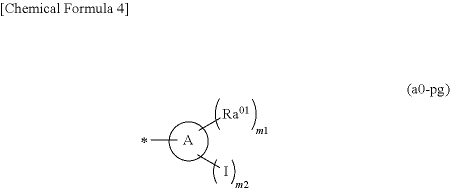

[In Formula (a0-p A represents a cyclic alkene having only one carbon-carbon unsaturated bond in a ring skeleton. Ra⁰¹ represents a monovalent hydrocarbon group having 1 to 20 carbon atoms. I represents an iodine atom. m1 represents an integer of 0 to 20. m2 represents an integer of 1 to 20. Here, m1+m2≤(a total number of carbon atoms constituting the ring skeleton of the cyclic alkene as A-1) is satisfied. In a case where m1 represents an integer of 2 or greater, a plurality of Ra⁰¹'s may be the same as or different from each other. $ represents a bonding site that is bonded to —O— (oxy group) in —Yb⁰—CO—O—.]

According to a fourth aspect of the present invention, there is provided a compound represented by General Formula (b0-p).

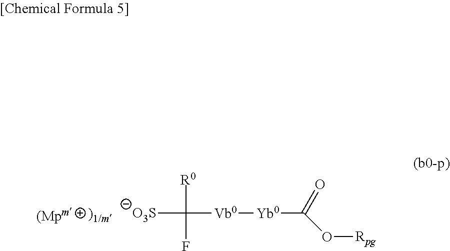

[In Formula (b0-p), Mp^{m′+} represents an organic cation or a metal cation. m′ represents an integer of 1 or greater. R⁰ represents a fluorinated alkyl group having 1 to carbon atoms or a fluorine atom. Vb⁰ represents an alkylene group, a fluorinated alkylene group, or a single bond. Yb⁰ represents a divalent linking group. Rpg represents an acid dissociable group represented by General Formula (a0-pg).]

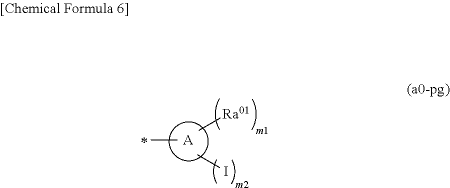

[In Formula (a0-pg), A represents a cyclic alkene having only one carbon-carbon unsaturated bond in a ring skeleton. Ra⁰¹ represents a monovalent hydrocarbon group having 1 to 20 carbon atoms. I represents an iodine atom. m1 represents an integer of 0 to 20. m2 represents an integer of 1 to 20. Here, m1+m2≤(a total number of carbon atoms constituting the ring skeleton of the cyclic alkene as A-1) is satisfied. In a case where m1 represents an integer of 2 or greater, a plurality of Ra⁰¹'s may be the same as or different from each other. * represents a bonding site that is bonded to —O— (oxy group) in —Yb⁰—CO—O—.]

### Advantageous Effects of Invention

According to the present invention, it is possible to provide a resist composition capable of achieving high sensitivity in a case of formation of a resist pattern and suppressing a decrease in film thickness during development, a resist pattern formation method using the resist composition, and a compound and an intermediate thereof, which are useful as an acid generator component used in the resist composition.

### DESCRIPTION OF EMBODIMENTS

In the present specification and the scope of the present claims, the term “aliphatic” is a relative concept used in relation to the term “aromatic”, and defines a group, a compound, or the like that has no aromaticity.

The term “alkyl group” includes a linear, branched, or cyclic monovalent saturated hydrocarbon group unless otherwise specified. The same applies to the alkyl group in an alkoxy group.

The term “alkylene group” includes a linear, branched, or cyclic divalent saturated hydrocarbon group unless otherwise specified.

Examples of “halogen atom” include a fluorine atom, a chlorine atom, a bromine atom, and an iodine atom.

The term “constitutional unit” indicates a monomer unit constituting a polymer compound (a resin, a polymer, or a copolymer).

The expression “may have a substituent” includes both a case where a hydrogen atom (—H) is substituted with a monovalent group and a case where a methylene group (—CH₂—) is substituted with a divalent group.

The term “light exposure” is a general concept for irradiation with radiation.

The term “acid decomposable group” indicates a group having acid decomposability in which at least a part of a bond in the structure of the acid decomposable group can be cleaved by the action of an acid.

Examples of the acid decomposable group whose polarity is increased by the action of an acid include groups which are decomposed by the action of an acid to generate a polar group.

Examples of the polar group include a carboxy group, a hydroxyl group, an amino group, and a sulfo group (—SO₃H).

More specific examples of the acid decomposable group include a group in which the above-described polar group has been protected by an acid dissociable group (such as a group in which a hydrogen atom of the OH-containing polar group has been protected by an acid dissociable group).

Here, the term “acid dissociable group” indicates both a group (i) having an acid dissociation property in which a bond between the acid dissociable group and an atom adjacent to the acid dissociable group can be cleaved by the action of an acid and a group (ii) in which some bonds are cleaved by the action of an acid, a decarboxylation reaction occurs, and thus the bond between the acid dissociable group and the atom adjacent to the acid dissociable group can be cleaved.

It is necessary that the acid dissociable group that constitutes the acid decomposable group is a group which exhibits a lower polarity than that of the polar group generated by the dissociation of the acid dissociable group. Thus, in a case where the acid dissociable group is dissociated by the action of an acid, a polar group exhibiting a higher polarity than that of the acid dissociable group is generated so that the polarity is increased. As a result, the polarity of the entire base material component increases. Due to the increase in the polarity, the solubility in a developing solution is relatively changed such that the solubility is increased in a case where the developing solution is an alkali developing solution and the solubility is decreased in a case where the developing solution is an organic developing solution.

The term “base material component” denotes an organic compound having a film-forming ability. Organic compounds used as the base material component are classified into non-polymers and polymers. As the non-polymers, typically non-polymers having a molecular weight of 500 or greater and less than 4000 (hereinafter, referred to as “low-molecular-weight compounds”) are used. Hereinafter, “resin”, “polymer compound”, or “polymer” indicates a polymer having a molecular weight of 1000 or greater. As the molecular weight of the polymer, the weight-average molecular weight in terms of polystyrene according to gel permeation chromatography (GPC) is used.

The expression “constitutional unit to be derived” denotes a constitutional unit formed by cleavage of a multiple bond between carbon atoms, for example, an ethylenic double bond.

In “acrylic acid ester”, the hydrogen atom bonded to the carbon atom at the α-position may be substituted with a substituent. The substituent (R^{αx}) that substitutes the hydrogen atom bonded to the carbon atom at the α-position is an atom other than the hydrogen atom or a group. Further, itaconic acid diester in which the substituent (R^{αx}) has been substituted with a substituent having an ester bond or α-hydroxyacryl ester in which the substituent (R^{αx}) has been substituted with a hydroxyalkyl group or a group obtained by modifying a hydroxyl group thereof can be described as acrylic acid ester. Further, the carbon atom at the α-position of acrylic acid ester indicates the carbon atom to which the carbonyl group of acrylic acid is bonded, unless otherwise specified.

Hereinafter, acrylic acid ester in which the hydrogen atom bonded to the carbon atom at the α-position has been substituted with a substituent is also referred to as α-substituted acrylic acid ester.

The concept “derivative” includes those obtained by substituting a hydrogen atom at the α-position of a target compound with another substituent such as an alkyl group or a halogenated alkyl group, and derivatives thereof. Examples of the derivatives thereof include those obtained by substituting a hydrogen atom of a hydroxyl group of a target compound, in which the hydrogen atom at the α-position may be substituted with a substituent, with an organic group, and those obtained by bonding a substituent other than a hydroxyl group to a target compound in which the hydrogen atom at the g-position may be substituted with a substituent. Further, the α-position denotes the first carbon atom adjacent to a functional group unless otherwise specified.

Examples of the substituent that substitutes the hydrogen atom at the α-position of hydroxystyrene include those for R^{αx}.

In the present specification and the scope of the present claims, asymmetric carbons may be present and enantiomers or diastereomers may be present depending on the structures of the chemical formulae. In this case, these isomers are represented by one chemical formula. These isomers may be used alone or in the form of a mixture.

### (Resist Composition)

The resist composition according to the first aspect of the present invention is a resist composition that generates an acid upon light exposure and whose solubility in a developing solution is changed by the action of the acid.

According to one embodiment, such a resist composition contains a base material component (A) whose solubility in a developing solution is changed by the action of the acid (hereinafter, also referred to as “component (A)”), and an acid generator component (B) that generates an acid upon light exposure (hereinafter, also referred to as “component (B)”). In the resist composition of the present embodiment, a compound (B0) represented by General Formula (b0) is used as the component (B).

In a case where a resist film is formed using the resist composition according to the present embodiment and the formed resist film is subjected to selective exposure, an acid is generated from the component (B) at exposed portions of the resist film, and the generated acid acts on the component. (A) to change the solubility of the component (A) in a developing solution, whereas the solubility of the component (A) in a developing solution is not changed at unexposed portions of the resist film, and thus a difference in solubility in the developing solution occurs between the exposed portions and the unexposed portions of the resist film. Therefore, in a case where the resist film is developed, the exposed portion of the resist film is dissolved and removed to form a positive-tone resist pattern in a case where the resist composition is of a positive-tone, whereas the unexposed portion of the resist film is dissolved and removed to form a negative-tone resist pattern in a case where the resist composition is of a negative tone.

The resist composition of the present embodiment may be a positive-tone resist composition or a negative-tone resist composition. Further, in the formation of a resist pattern, the resist composition according to the present embodiment can be applied to an alkali developing process using an alkali developing solution in the developing treatment, or a solvent developing process using a developing solution containing an organic solvent (organic developing solution) in the developing treatment.

### <Component (A): Base Material Component>

In the resist composition according to the present embodiment, it is preferable that the component (A) has a resin component (A1) whose solubility in a developing solution is changed by the action of an acid (hereinafter, also referred to as “component (A1)”). Since the polarity of the base material component before and after the light exposure is changed by using the component (A1), an excellent development contrast can be obtained not only in an alkali developing process but also in a solvent developing process.

As the component (A), at least one of other polymer compounds or low-molecular-weight compounds may be used in combination with the component (A1).

In the resist composition according to the present embodiment, the component (A) may be used alone or in combination of two or more kinds thereof.

### In Regard to Component (A1)

The component (A1) is a resin component whose solubility in a developing solution is changed by the action of an acid.

As the component (A1), those having a constitutional unit (a1) containing an acid decomposable group whose polarity is increased by the action of an acid are preferable. Further, the component (A1) may have other constitutional units as necessary in addition to the constitutional unit (a1).

Examples of the other constitutional units include a constitutional unit (a10) represented by General Formula (a10-1) described later; a constitutional unit (a2) containing a lactone-containing cyclic group; and a constitutional unit (a8) derived from a compound represented by General Formula (a8-1) described later.

### In Regard to Constitutional Unit (a1):

The constitutional unit (a1) is a constitutional unit that contains an acid decomposable group whose polarity is increased by the action of an acid.

Examples of the acid decomposable group include a group in which a polar group is protected by an acid dissociable group (for example, a group in which a hydrogen atom of an OH-containing polar group is protected by an acid dissociable group).

Examples of the acid dissociable group are the same as those which have been suggested as the acid dissociable groups of the base resin for a chemically amplified resist composition.

Specific examples of the acid dissociable group of the base resin suggested for a chemically amplified resist composition include “acetal type acid dissociable group”, “tertiary alkyl ester type acid dissociable group”, “tertiary alkyloxycarbonyl acid dissociable group”, and “secondary alkyl ester type acid dissociable group” described below.

### Acetal Type Acid Dissociable Group;

Examples of the acid dissociable group that protects a carboxy group or a hydroxyl group in the polar groups include an acid dissociable group represented by General Formula (a1-r-1) (hereinafter, also referred to as “acetal type acid dissociable group”).

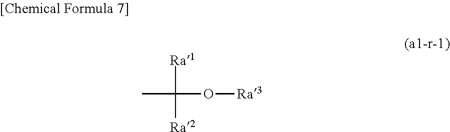

[In the formula, Ra′¹ and Ra′¹ represent a hydrogen atom or an alkyl group. Ra′³ represents a hydrocarbon group, and Ra′³ may be bonded to any of Ra′¹ and Ra′² to form a ring.]

In Formula (a1-r-1), it is preferable that at least one of Ra′¹ and Ra′² represents a hydrogen atom and more preferable that both Ra′¹ and Ra′² represent a hydrogen atom.

In a case where Ra′¹ or Ra′² represents an alkyl group, examples of the alkyl group include the same alkyl groups exemplified as the substituent which may be bonded to the carbon atom at the α-position in the description on α-substituted acrylic acid ester. Among these, an alkyl group having 1 to 5 carbon atoms is preferable. Specific preferred examples thereof include linear or branched alkyl groups. More specific examples thereof include a methyl group, an ethyl group, a propyl group, an isopropyl group, an n-butyl group, an isobutyl group, a tert-butyl group, a pentyl group, an isopentyl group, and a neopentyl group. Among these, a methyl group or an ethyl group is more preferable, and a methyl group is particularly preferable.

In Formula (a1-r-1), examples of the hydrocarbon group as Ra′³ include a linear or branched alkyl group and a cyclic hydrocarbon group.

The linear alkyl group has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group has preferably 3 to 10 carbon atoms and more preferably 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. Among these, an isopropyl group is preferable.

In a case where Ra′³ represents a cyclic hydrocarbon group, the hydrocarbon group may be an alicyclic hydrocarbon group or an aromatic hydrocarbon group and may be a polycyclic group or a monocyclic group.

The alicyclic hydrocarbon group which is a monocyclic group is preferably a group in which one hydrogen atom has been removed from a monocycloalkane. The monocycloalkane has preferably 3 to 6 carbon atoms, and specific examples thereof include cyclopentane and cyclohexane.

The alicyclic hydrocarbon group which is a polycyclic group is preferably a group in which one hydrogen atom has been removed from a polycycloalkane. The polycycloalkane preferably has 7 to 12 carbon atoms, and specific examples thereof include adamantane, norbornane, isobornane, tricyclodecane, and tetracyclododecane.

In a case where the cyclic hydrocarbon group as Ra′³ becomes an aromatic hydrocarbon group, the aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to carbon atoms, still more preferably 6 to 15 carbon atoms, and particularly preferably 6 to 12 carbon atoms.

Specifically, as the aromatic ring, an aromatic hydrocarbon ring such as benzene, naphthalene, anthracene, and phenanthrene; and an aromatic heterocyclic ring in which some carbon atoms constituting the aromatic hydrocarbon ring have been substituted with heteroatoms are exemplary examples. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group as Ra′³ include a group in which one hydrogen atom has been removed from the above-described aromatic hydrocarbon ring or aromatic heterocyclic ring (such as an aryl group or a heteroaryl group); a group in which one hydrogen atom has been removed from an aromatic compound having two or more aromatic rings (such as biphenyl or fluorene); and a group in which one hydrogen atom of the above-described aromatic hydrocarbon ring or aromatic heterocyclic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aromatic hydrocarbon ring or aromatic heterocyclic ring is preferably in a range of 1 to 4, more preferably 1 or 2, and particularly preferably 1.

The cyclic hydrocarbon group as Ra′³ may have a substituent. Examples of the substituent include —R^{P1}, —R^{P2}—O—R^{P1}, —R^{P2}—CO—R^{P1}, —R^{P2}—CO—OR^{P1}, —R^{P2}—O—COR^{P1}, R^{P2}—OH, —R^{P2}—CN, and —R^{P2}—COOH) (hereinafter, these substituents will also be collectively referred to as “Ra^{x5}”).

Here, R^{P1} represents a chain-like monovalent saturated hydrocarbon group having 1 to 10 carbon atoms, a monovalent aliphatic cyclic saturated hydrocarbon group having 3 to 20 carbon atoms, or a monovalent aromatic hydrocarbon group having 6 to carbon atoms. Further, R^{P2} represents a single bond, a chain-like divalent saturated hydrocarbon group having 1 to 10 carbon atoms, a divalent aliphatic cyclic saturated hydrocarbon group having 3 to 20 carbon atoms, or a divalent aromatic hydrocarbon group having 6 to 30 carbon atoms. Some or all hydrogen atoms in the chain-like saturated hydrocarbon group, the aliphatic cyclic saturated hydrocarbon group, and the aromatic hydrocarbon group as R^{P1} and R^{P2} may be substituted with fluorine atoms. The aliphatic cyclic hydrocarbon group may have one or more of one kind of substituents or one or more of each of plural kinds of the substituents.

Examples of the chain-like monovalent saturated hydrocarbon group having 1 to carbon atoms include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.02,6]decanyl group, a tricyclo[3.3.1.13,7] decanyl group, a tetracyclo[6.2.1.13,6.02,7]dodecanyl group, or an adamantyl group.

Examples of the monovalent aromatic hydrocarbon group having 6 to 30 carbon atoms include a group formed by removing one hydrogen atom from an aromatic hydrocarbon ring such as benzene, biphenyl, fluorene, naphthalene, anthracene, or phenanthrene.

In a case where Ra′³ is bonded to any of Ra′¹ and Ra′² to form a ring, the cyclic group is preferably a 4- to 7-membered ring and more preferably a 4- to 6-membered ring. Specific examples of the cyclic group include a tetrahydropyranyl group and a tetrahydrofuranyl group.

Tertiary alkyl ester type acid dissociable group:

Examples of the acid dissociable group that protects a carboxy group among the polar groups include an acid dissociable group represented by General Formula (a1-r-2).

Among examples of the acid dissociable group represented by Formula (a1-r-2), a group formed of an alkyl group is referred to as “tertiary alkyl ester type acid dissociable group” for convenience,

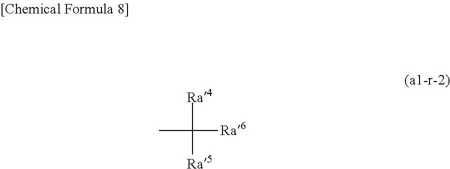

[In the formula, Ra′⁴ to Ra′⁶ each represents a hydrocarbon group, and Ra′⁵ and Ra′⁶ may be bonded to each other to form a ring.]

Examples of the hydrocarbon group as Ra′⁴ include a linear or branched alkyl group, a chain-like or cyclic alkenyl group, a chain-like alkynyl group, and a cyclic hydrocarbon group.

Examples of the linear or branched alkyl group and the cyclic hydrocarbon group (an alicyclic hydrocarbon group which is a monocyclic group, an alicyclic hydrocarbon group which is a polycyclic group, or an aromatic hydrocarbon group) as Ra′⁴ include the same groups as those for Ra′³.

As the chain-like or cyclic alkenyl group as Ra′⁴, an alkenyl group having 2 to carbon atoms is preferable.

Examples of the hydrocarbon group as Ra′⁵ or Ra′⁶ include the same groups as those for Ra′³.

In a case where Ra′⁵ and Ra′⁶ are bonded to each other to form a ring, suitable examples thereof include a group represented by General Formula (a1-r2-1), a group represented by General Formula (a1-+2-2), and a group represented by General Formula (a1+2-3).

Meanwhile, in a case where Ra′⁴ to Ra′⁶ represent an independent hydrocarbon group without being bonded to one another, suitable examples thereof include a group represented by General Formula (a1-r2-4).

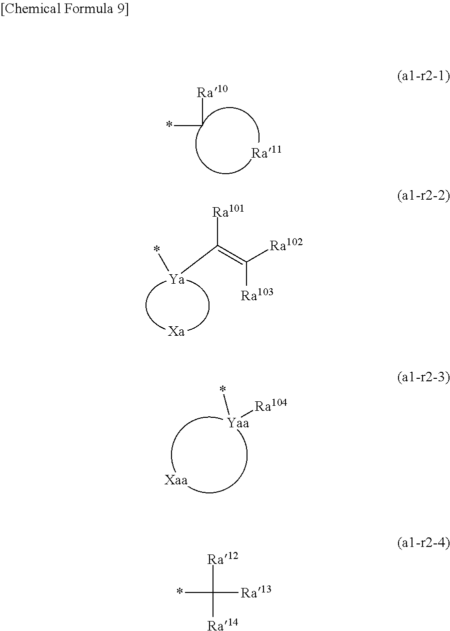

[In Formula (a1-r2-1), Rate represents a linear or branched alkyl group having 1 to 12 carbon atoms, in which a part thereof may be substituted with a halogen atom or a heteroatom-containing group. Ra′¹¹ represents a group that forms an aliphatic cyclic group with the carbon atom to which Ra′¹⁰ has been bonded. In Formula (a1-r2-2), Ya represents a carbon atom. Xa represents a group that forms a cyclic hydrocarbon group with Ya. Some or all hydrogen atoms in this cyclic hydrocarbon group may be substituted. Ra¹⁰¹ to Ra¹⁰³ each independently represent a hydrogen atom, a chain-like monovalent saturated hydrocarbon group having 1 to 10 carbon atoms, or a monovalent aliphatic cyclic saturated hydrocarbon group having 3 to 20 carbon atoms. Some or all hydrogen atoms in the chain-like saturated hydrocarbon group and the aliphatic cyclic saturated hydrocarbon group may be substituted. Two or more of Ra¹⁰¹ to Ra¹⁰³ may be bonded to one another to form a cyclic structure. In Formula (a1-r2-3). Yaa represents a carbon atom. Xaa represents a group that forms an aliphatic cyclic group with Yaa. Ra¹⁰⁴ represents an aromatic hydrocarbon group which may have a substituent. In Formula (a1-r2-4), Ra′¹² and Ra′¹³ each independently represent a monovalent chain-like saturated hydrocarbon group having 1 to 10 carbon atoms. Some or all of hydrogen atoms contained in the chain-like saturated hydrocarbon group may be substituted. Ra′¹⁴ represents a hydrocarbon group which may have a substituent. * represents a bonding site (the same applies hereinafter).]

In Formula (a1-r2-1), Ra′¹⁰ represents a linear or branched alkyl group having 1 to 12 carbon atoms, in which a part thereof may be substituted with a halogen atom or a heteroatom-containing group.

The linear alkyl group as Ra′¹⁰ has 1 to 12 carbon atoms, preferably 1 to 10 carbon atoms, and particularly preferably 1 to 5 carbon atoms.

Examples of the branched alkyl group as Ra′¹⁰ include those for Ra′³ described above.

The alkyl group in Ra′¹⁰ may be partially substituted with a halogen atom or a heteroatom-containing group. For example, some hydrogen atoms constituting the alkyl group may be substituted with a halogen atom or a heteroatom-containing group. Further, some carbon atoms (methylene group or the like) constituting the alkyl group may be substituted with a heteroatom-containing group.

Examples of the heteroatoms here include an oxygen atom, a nitrogen atom, and a sulfur atom. Examples of the heteroatom-containing group include (—O—), —C(═O)—O—, —O—C(═O)—, —C(═O)—, —O—C(═O)—O—, —C(═O)—NH—, —NH—, —S—, —S(═O)₂—, and —S(═O)₂—O—.

In Formula (a1-r2-1), preferred examples of Ra′¹¹ (an aliphatic cyclic group that is formed together with a carbon atom to which Ra′¹⁰ is bonded) include the groups described as the alicyclic hydrocarbon group (alicyclic hydrocarbon group) which is a monocyclic group or a polycyclic group as Ra′³ in Formula (a1-r-1). Among these, it is preferably a monocyclic alicyclic hydrocarbon group, and specifically, it is more preferably a cyclopentyl group or a cyclohexyl group.

In Formula (a1-r2-2), examples of the cyclic hydrocarbon group that is formed by Xa together with Ya include a group in which one or more hydrogen atoms have been further removed from the cyclic monovalent hydrocarbon group (alicyclic hydrocarbon group) as Ra′³ in Formula (a1-r-1).

The cyclic hydrocarbon group that is formed by Xa together with Ya may have a substituent. Examples of the substituent include those which are the same as the substituents which may be included in the cyclic hydrocarbon group as Ra′³.

In Formula (a1-r2-2), examples of the chain-like monovalent saturated hydrocarbon group having 1 to 10 carbon atoms as Ra¹⁰¹ to Ra¹⁰³ include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms as Ra¹⁰¹ to Ra¹⁰³ include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.02,6]decanyl group, a tricyclo[3.3.1.13,7] decanyl group, a tetracyclo[6.2.1.13,6.02.7] dodecanyl group, or an adamantyl group.

From the viewpoint of ease of synthesis, Ra¹⁰¹ to Ra¹⁰³ represent preferably a hydrogen atom or a chain-like monovalent saturated hydrocarbon group having 1 to 10 carbon atoms, more preferably a hydrogen atom, a methyl group, or an ethyl group, and particularly preferably a hydrogen atom.

Examples of the substituent included in the chain-like saturated hydrocarbon group or the aliphatic cyclic saturated hydrocarbon group represented by Ra¹⁰¹ to Ra¹⁰³ are the same as those for Ra^{x5}.

Examples of the group having a carbon-carbon double bond generated by two or more of Ra¹⁰¹ to Ra¹⁰³ being bonded to one another to form a cyclic structure include a cyclopentenyl group, a cyclohexenyl group, a methylcyclopentenyl group, a methylcyclohexenyl group, a cyclopentylidenethenyl group, and a cyclohexylidenethenyl group. Among these, from the viewpoint of ease of synthesis, a cyclopentenyl group, a cyclohexenyl group, or a cyclopentylidenethenyl group is preferable.

In Formula (a1-r2-3), as the aliphatic cyclic group that is formed by Xaa together with Yaa, the group described as the alicyclic hydrocarbon group which is a monocyclic group or a polycyclic group as Ra′³ in Formula (a1-r-1) is preferable.

In Formula (a1-r2-3), examples of the aromatic hydrocarbon group as Ra¹⁰⁴ include a group in which one or more hydrogen atoms have been removed from an aromatic hydrocarbon ring having 5 to 30 carbon atoms. Among the examples, Ra¹⁰⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from benzene or naphthalene, and most preferably a group in which one or more hydrogen atoms have been removed from benzene.

Examples of the substituent which may be included in Ra¹⁰⁴ in Formula (a1-r2-3) include a methyl group, an ethyl group, a propyl group, a hydroxyl group, a carboxyl group, a halogen atom, an alkoxy group (such as a methoxy group, an ethoxy group, a propoxy group, or a butoxy group), and an alkyloxycarbonyl group.

In Formula (a1-r2-4), Ra′¹² and Ra′¹³ each independently represent a monovalent chain-like saturated hydrocarbon group having 1 to 10 carbon atoms. Examples of the monovalent chain-like saturated hydrocarbon group having 1 to 10 carbon atoms as Ra′¹² and Ra′¹³ include the same one as the monovalent chain-like saturated hydrocarbon group having 1 to 10 carbon atoms as Ra¹⁰¹ to Ra¹⁰³ as described above. Some or all of hydrogen atoms contained in the chain-like saturated hydrocarbon group may be substituted.

Ra′¹² and Ra′¹³ represent preferably an alkyl group having 1 to 5 carbon atoms, more preferably an alkyl group having 1 to 5 carbon atoms, still more preferably a methyl group or an ethyl group, and particularly preferably a methyl group.

In a case where the chain-like saturated hydrocarbon group represented by Ra′¹² and Ra′¹³ is substituted, examples of the substituent are those for Ra^{x5} described above.

In Formula (a1-r2-4), Ra′¹⁴ represents a hydrocarbon group which may have a substituent. Examples of the hydrocarbon group as Ra′¹⁴ include a linear or branched alkyl group and a cyclic hydrocarbon group.

The linear alkyl group as Ra′¹⁴ has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group as Ra′¹⁴ has preferably 3 to 10 carbon atoms and more preferably 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. Among these, an isopropyl group is preferable.

In a case where Ra′¹⁴ represents a cyclic hydrocarbon group, the hydrocarbon group may be an alicyclic hydrocarbon group or an aromatic hydrocarbon group and may be a polycyclic group or a monocyclic group.

The alicyclic hydrocarbon group which is a monocyclic group is preferably a group in which one hydrogen atom has been removed from a monocycloalkane. The monocycloalkane has preferably 3 to 6 carbon atoms, and specific examples thereof include cyclopentane and cyclohexane.

The alicyclic hydrocarbon group which is a polycyclic group is preferably a group in which one hydrogen atom has been removed from a polycycloalkane. The polycycloalkane preferably has 7 to 12 carbon atoms, and specific examples thereof include adamantane, norbornane, isobornane, tricyclodecane, and tetracyclododecane.

Examples of the aromatic hydrocarbon group as Ra′¹⁴ include the same groups as those for the aromatic hydrocarbon group as Ra¹⁰⁴. Among these, Ra′¹⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from naphthalene or anthracene, and most preferably a group in which one or more hydrogen atoms have been removed from naphthalene.

Examples of the substituent which may be included in Ra′¹⁴ include the same groups as those for the substituent which may be included in Ra¹⁰⁴.

In a case where Ra′¹⁴ in Formula (a1-r2-4) represents a naphthyl group, the position bonded to the tertiary carbon atom in Formula (a1-r2-4) may be the 1-position or the 2-position of the naphthyl group.

In a case where Ra′¹⁴ in Formula (a1-12-4) represents an anthryl group, the position bonded to the tertiary carbon atom in Formula (a1-r2-4) may be the 1-position, the 2-position, or the 9-position of the anthryl group.

Specific examples of the group represented by Formula (a1-r2-1) are shown below.

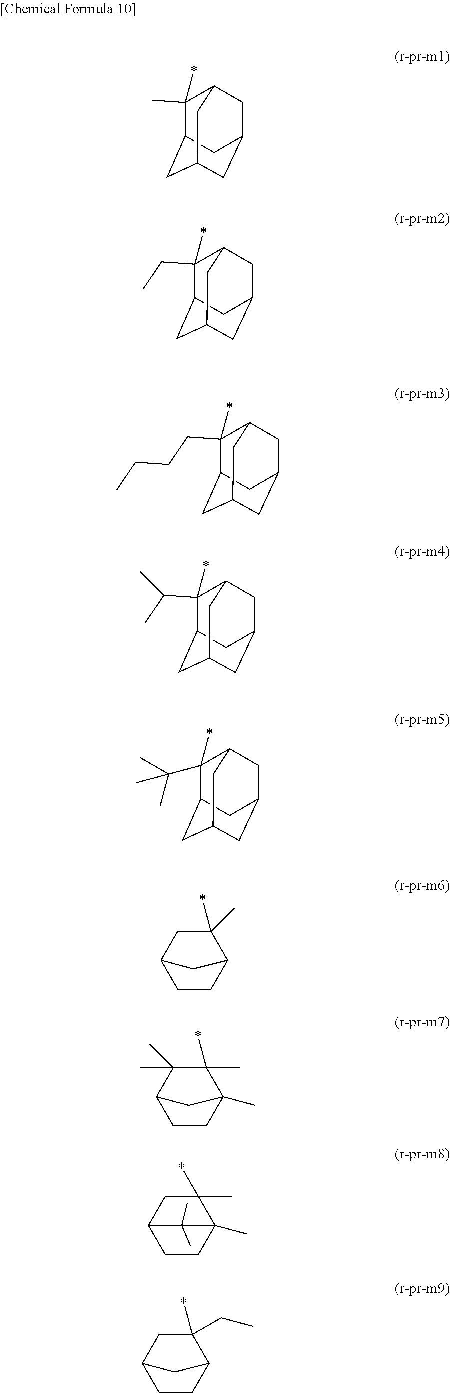

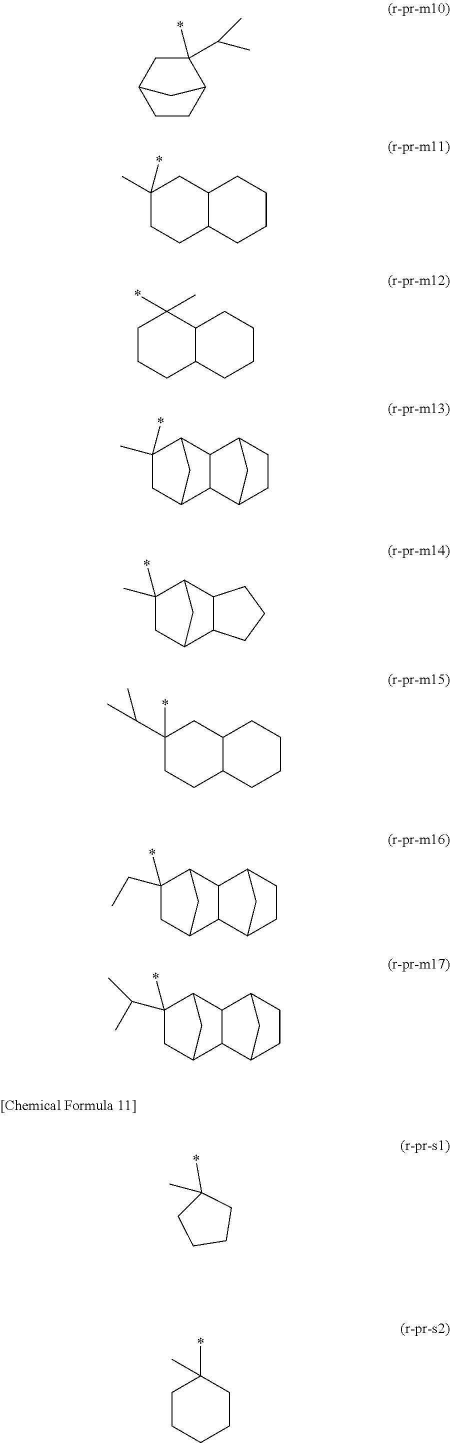

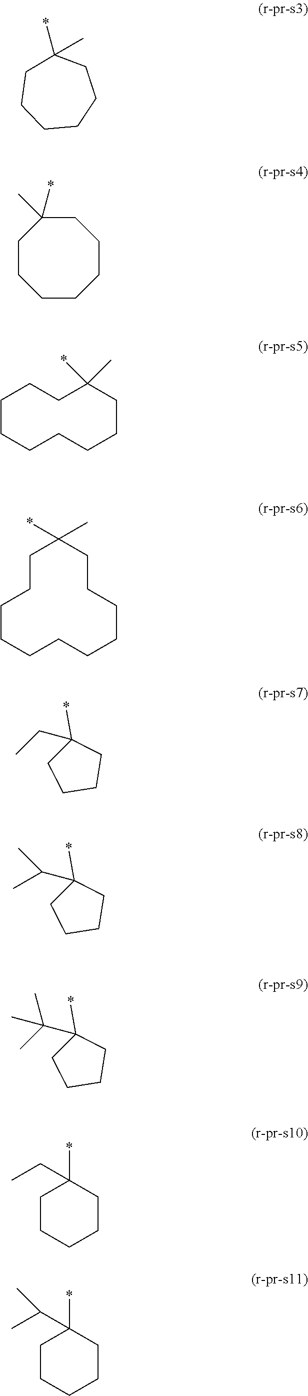

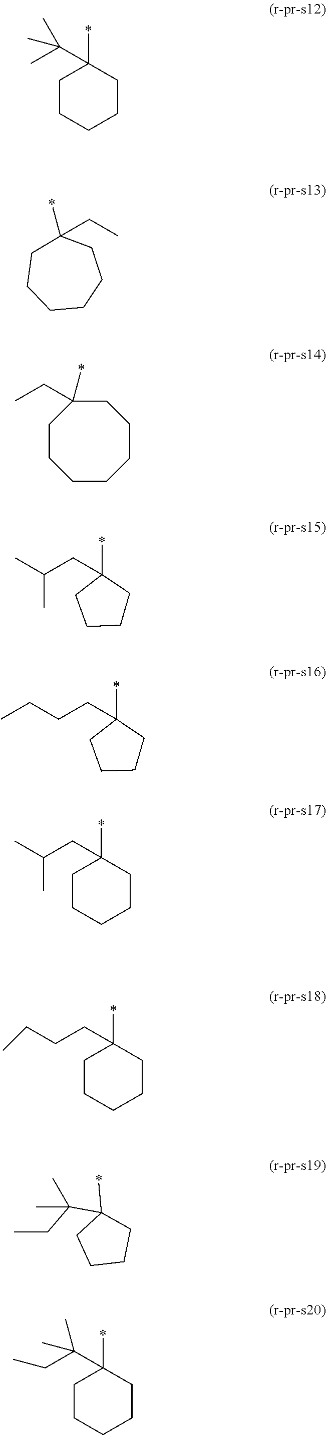

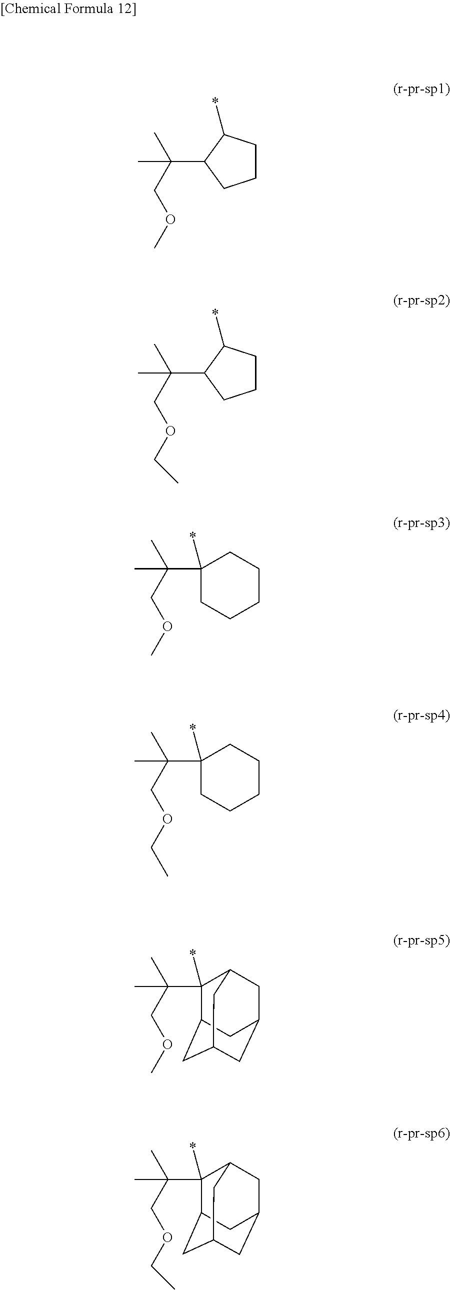 [SMILES: *C1CCCC1C(C)(C)COC ; *C1CCCC1C(C)(C)COCC ; *C1(C(C)(C)COC)CCCCC1 ; *C1(C(C)(C)COCC)CCCCC1 ; *C1(C(C)(C)COC)C2CC3CC(C2)CC1C3 ; *C1(C(C)(C)COCC)C2CC3CC(C2)CC1C3]

Specific examples of the group represented by Formula (a1-r2-2) are shown below.

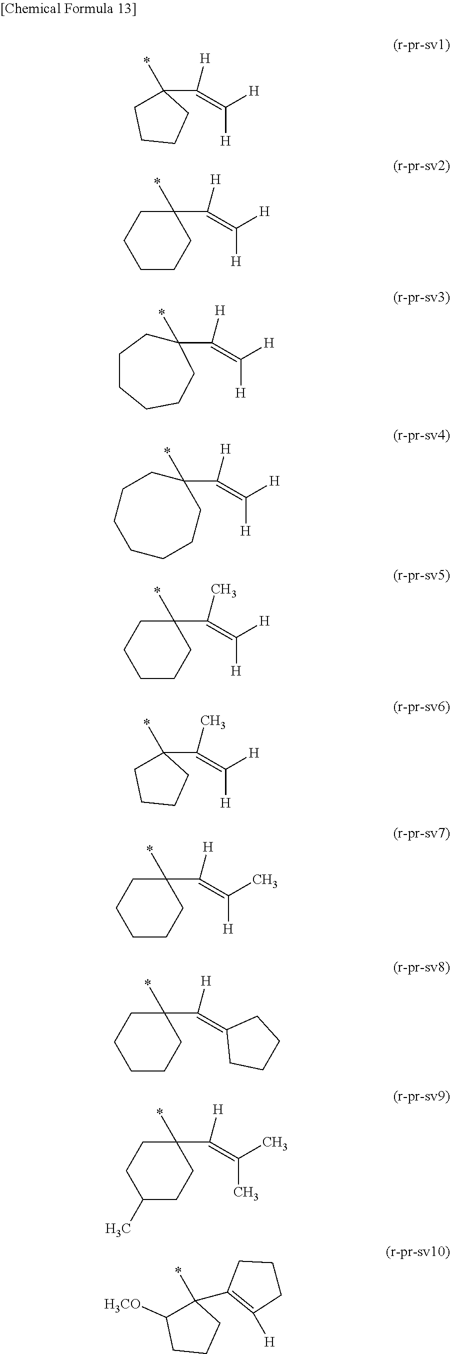

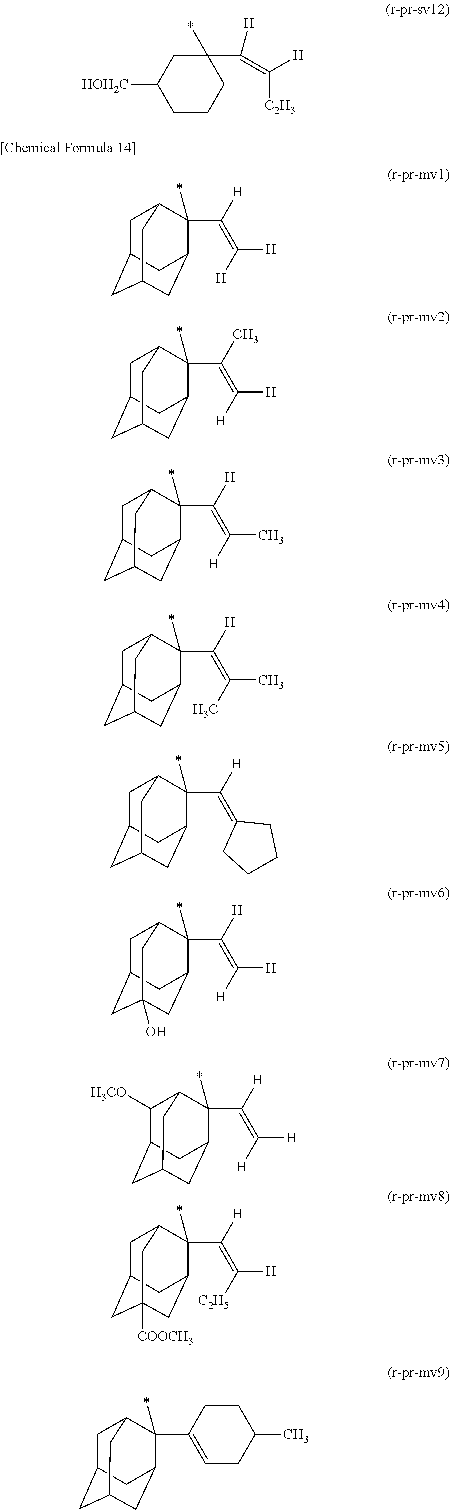

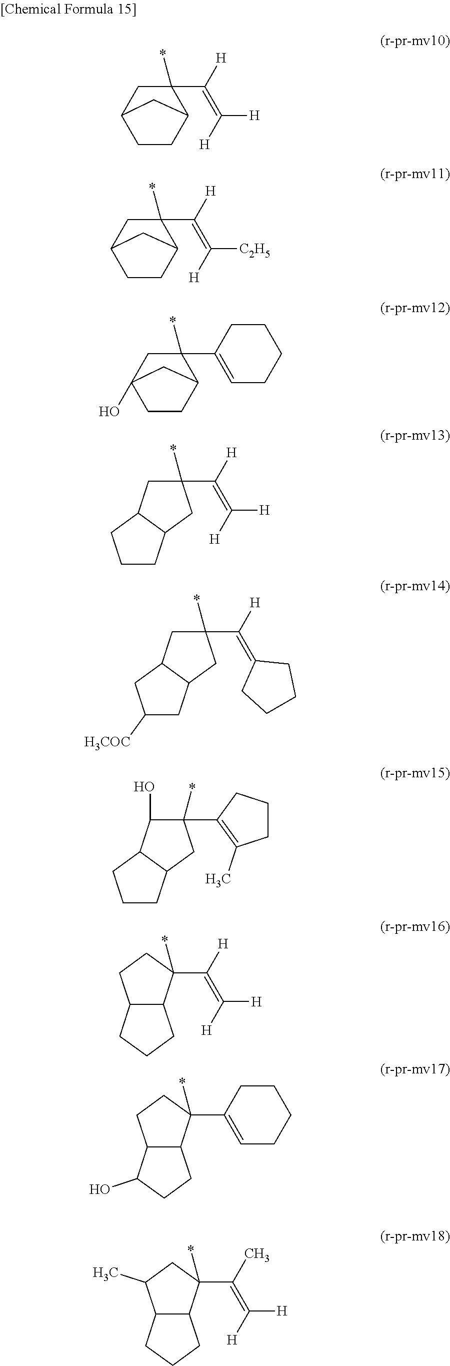

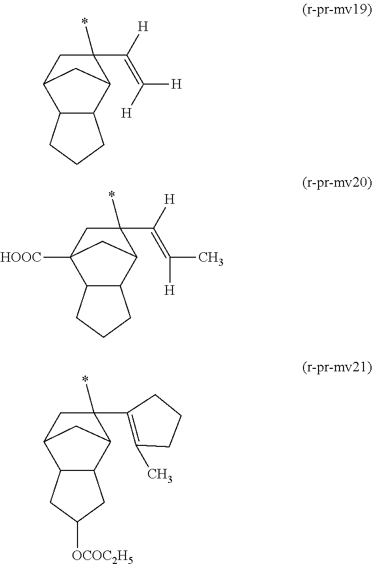

Specific examples of the group represented by Formula (a1-r2-3) are shown below.

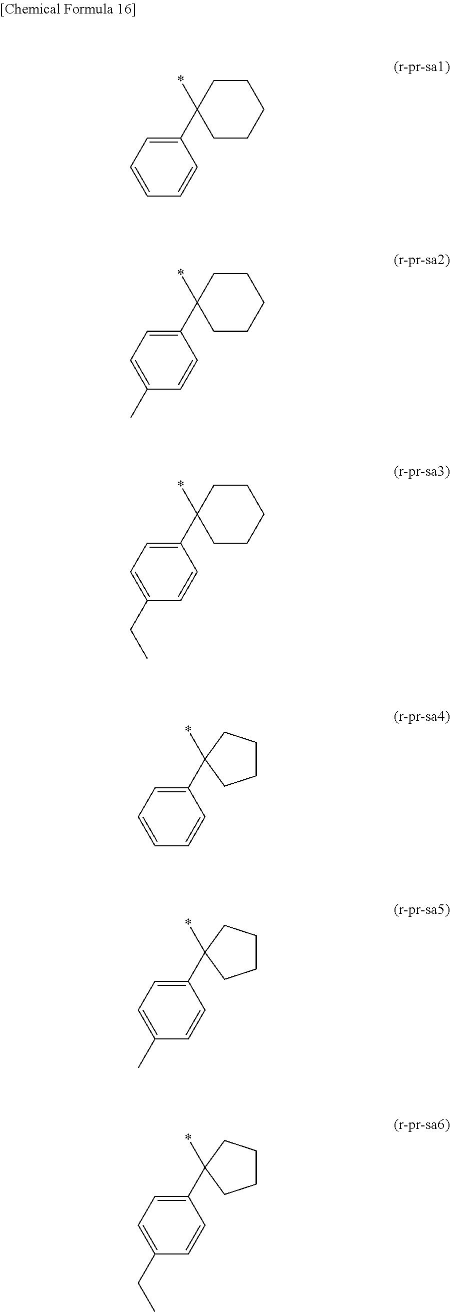 [SMILES: *C1(c2ccccc2)CCCCC1 ; *C1(c2ccc(C)cc2)CCCCC1 ; *C1(c2ccc(CC)cc2)CCCCC1 ; *C1(c2ccccc2)CCCC1 ; *C1(c2ccc(C)cc2)CCCC1 ; *C1(c2ccc(CC)cc2)CCCC1]

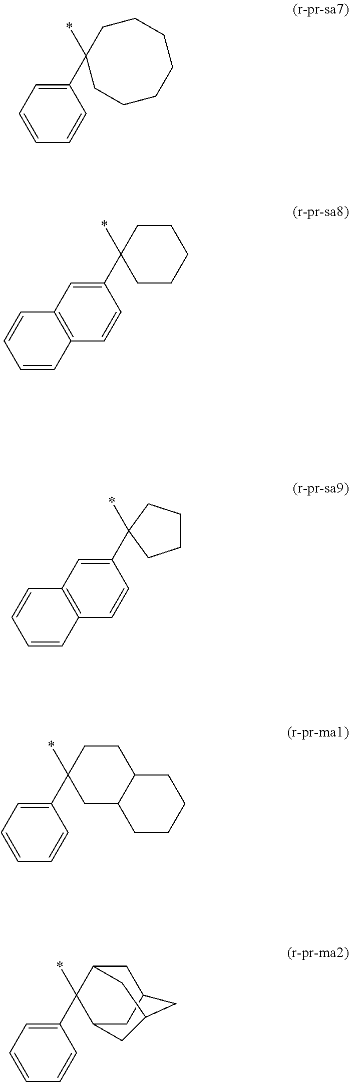 [SMILES: *C1(c2ccccc2)CCCCCCC1 ; *C1(c2ccc3ccccc3c2)CCCCC1 ; *C1(c2ccc3ccccc3c2)CCCC1 ; *C1(c2ccccc2)CCC2CCCCC2C1 ; *C1(c2ccccc2)C2CC3CC(C2)CC1C3]

Specific examples of the group represented by Formula (a1-r2-4) are shown below.

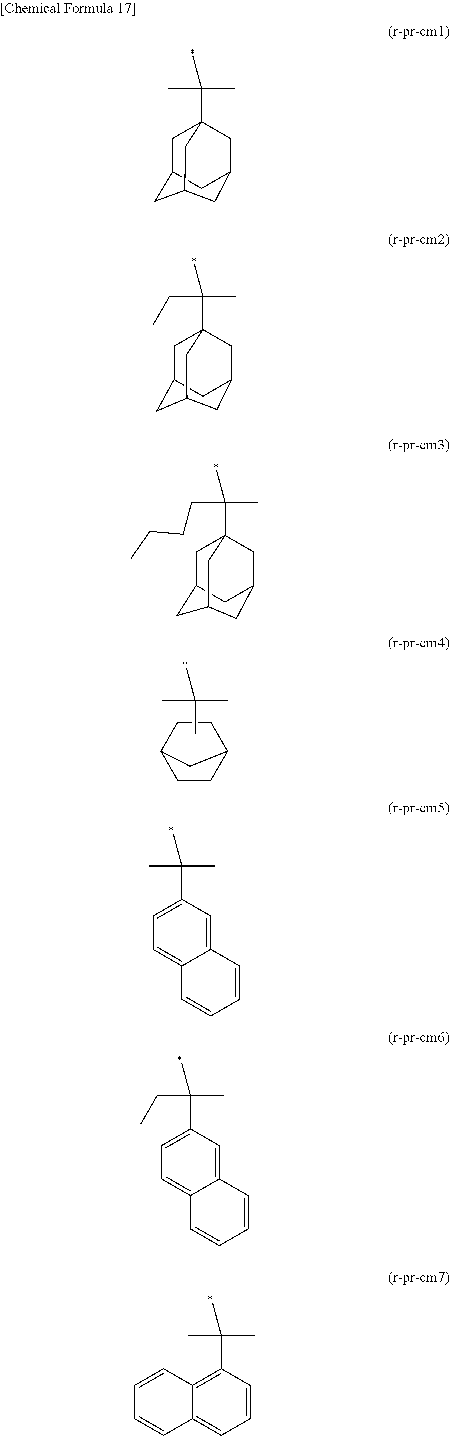

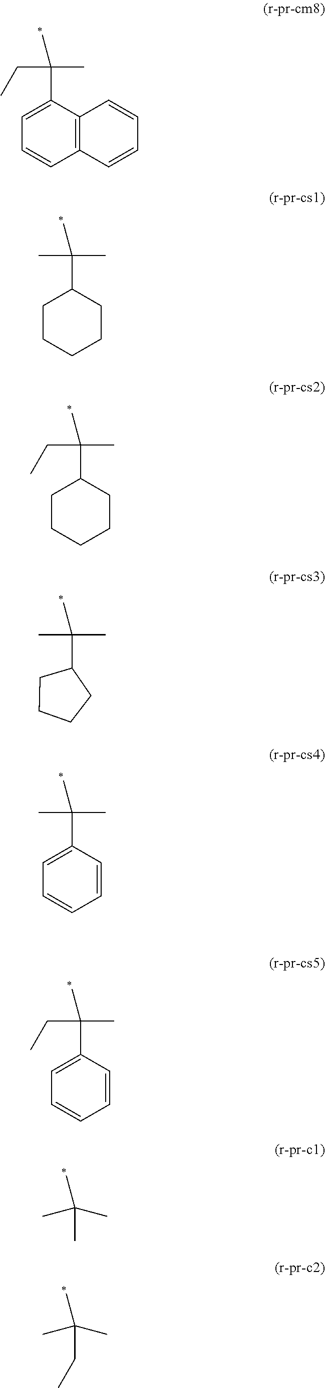

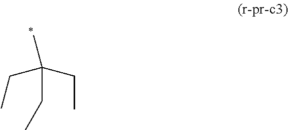

### Tertiary Alkyloxycarbonyl Acid Dissociable Group:

Examples of the acid dissociable group that protects a hydroxyl group among the polar groups include an acid dissociable group (hereinafter, also referred to as “tertiary alkyloxycarbonyl acid dissociable group” for convenience) represented by General Formula (a1-r-3).

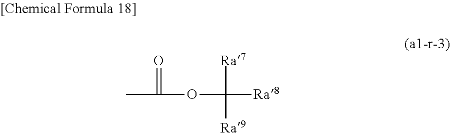

[In the formula, Ra′⁷ to Ra′⁹ each independently represent an alkyl group.]

In Formula (a1-r-3), Ra′⁷ to Ra′⁹ each represent preferably an alkyl group having 1 to 5 carbon atoms and more preferably an alkyl group having 1 to 3 carbon below.

Further, the total number of carbon atoms in each alkyl group is preferably in a range of 3 to 7, more preferably in a range of 3 to 5, and most preferably 3 or 4.

### Secondary Alkyl Ester Type Acid Dissociable Group:

Examples of the acid dissociable group that protects a carboxy group among the polar groups include an acid dissociable group represented by General Formula (a1-r-4).

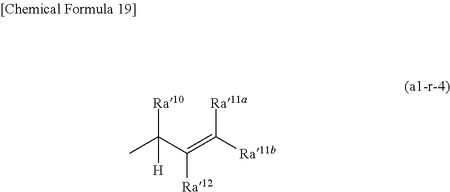

[In the formula, Ra′¹⁰ represents a hydrocarbon group. Ra′^{11a} and Ra′^{11b} each independently represent a hydrogen atom, a halogen atom, or an alkyl group. Ra′¹² represents a hydrogen atom or a hydrocarbon group. Ra′¹⁰ and Ra′^{11a} or Ra′^{11b} may be bonded to each other to form a ring. Ra′^{11a} or Ra′^{11b} and Ra′¹² may be bonded to each other to form a ring.]

Examples of the hydrocarbon group as Ra′³⁰ or Ra′¹² in the formula include the same groups as those for Ra′³.

Examples of the alkyl group as Ra′^{11a} and Ra′^{11b} in the formula include the same groups as those for the alkyl group as Ra′¹.

In the formula, the hydrocarbon group as Ra′¹⁰ or Ra′¹² and the alkyl group as Ra′^{11a} Ha and Ra′^{11b} may have a substituent. Examples of this substituent include Ra^{x5} described above.

Ra′¹⁰ and Ra′^{11a} or Ra′^{11b} may be bonded to each other to form a ring. The ring may be a polycyclic ring or a monocyclic ring, and may be an alicyclic ring or an aromatic ring.

The alicyclic ring and the aromatic ring may have a heteroatom.

Among the examples described above, as the ring formed by Ra′¹⁰ and Ra′^{11a} or Ra′^{11b} being bonded to each other, monocycloalkene, a ring in which some carbon atoms of monocycloalkene are substituted with heteroatoms (such as an oxygen atom and a sulfur atom), or monocycloalkadiene is preferable, cycloalkene having 3 to 6 carbon atoms is preferable, and cyclopentene or cyclohexene is preferable.

The ring formed by Ra′¹⁰ and Ra′^{11a} or Ra′^{11b} being bonded to each other may be a condensed ring. Specific examples of the condensed ring include indane.

The ring formed by Ra′¹⁰ and Ra′^{11a} or Ra′^{11b} being bonded to each other may have a substituent. Examples of this substituent include Ra^{x5} described above.

Ra′^{11a} or Ra′^{11b} and Ra′¹² may be bonded to each other to form a ring, and examples of the ring include the rings formed by Ra′¹⁰ and Ra′^{11a} or Ra′^{11b} being bonded to each other.

Specific examples of the group represented by Formula (a1-r-4) are shown below.

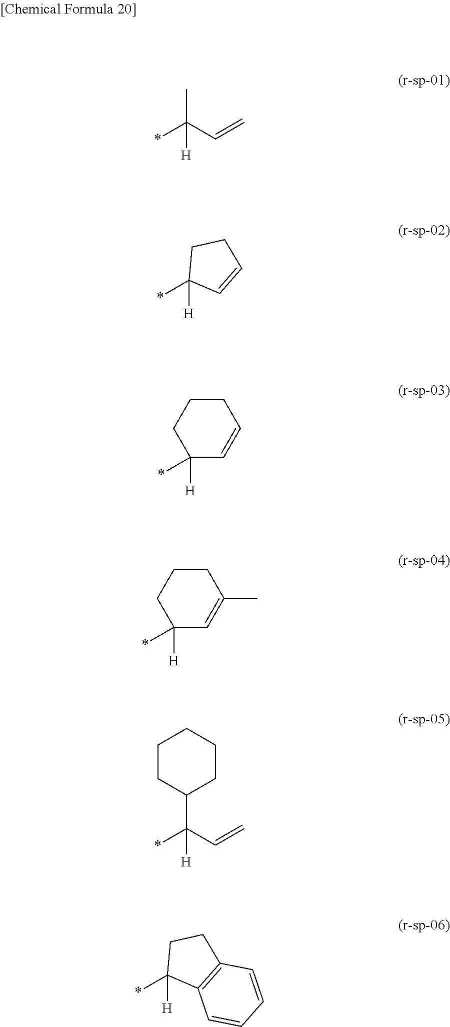

Examples of the constitutional unit (a1) include a constitutional unit derived from acrylic acid ester in which the hydrogen atom bonded to the carbon atom at the α-position may be substituted with a substituent; a constitutional unit derived from acrylamide; a constitutional unit in which at least some hydrogen atoms in a hydroxyl group of a constitutional unit derived from hydroxystyrene or a hydroxystyrene derivative are protected by a substituent containing the acid decomposable group; and a constitutional unit in which at least some hydrogen atoms in —C(═O)—OH of a constitutional unit derived from vinylbenzoic acid or a vinylbenzoic acid derivative are protected by a substituent containing the acid decomposable group.

Among the examples, as the constitutional unit (a1), a constitutional unit derived from acrylic acid ester in which the hydrogen atom bonded to the carbon atom at the α-position may be substituted with a substituent is preferable.

Specific preferred examples of such a constitutional unit (a1) include constitutional units represented by General Formula (a1-1) or (a1-2) shown below.

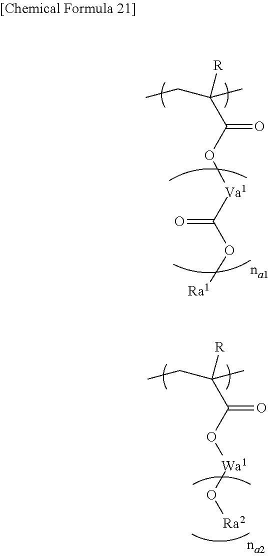

[In the formula, R represents a hydrogen atom, an alkyl group having 1 to 5 carbon atoms, or a halogenated alkyl group having 1 to 5 carbon atoms. Va¹ represents a divalent hydrocarbon group which may contain an ether bond. nₐ₁ represents an integer of 0 to 2. Ra¹ represents an acid dissociable group represented by General Formula (a1-r-1), (a1-r-2), or (a1-r-4). Wa¹ represents a (nₐ₂+1)-valent hydrocarbon group, nₐ₂ represents an integer of 1 to 3, and Ra² represents an acid dissociable group represented by General Formula (a1-r-1) or (a1-r-3)].

In Formula (a1-1), as the alkyl group having 1 to 5 carbon atoms as R, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all hydrogen atoms in the alkyl group having 1 to 5 carbon atoms have been substituted with halogen atoms. As the halogen atom, a fluorine atom is particularly preferable.

R represents preferably a hydrogen atom, an alkyl group having 1 to 5 carbon atoms, or a fluorinated alkyl group having 1 to 5 carbon atoms, and most preferably a hydrogen atom or a methyl group from the viewpoint of the industrial availability.

In Formula (a1-1), the divalent hydrocarbon group as Va¹ may be an aliphatic hydrocarbon group or an aromatic hydrocarbon group.

The aliphatic hydrocarbon group as the divalent hydrocarbon group represented by Va¹ may be saturated or unsaturated. In general, it is preferable that the aliphatic hydrocarbon group is saturated.

More specific examples of the aliphatic hydrocarbon group include a linear or branched aliphatic hydrocarbon group and an aliphatic hydrocarbon group having a ring in the structure thereof.

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

Examples of the aliphatic hydrocarbon group having a ring in the structure thereof include an alicyclic hydrocarbon group (a group in which two hydrogen atoms have been removed from an aliphatic hydrocarbon ring), a group in which the alicyclic hydrocarbon group is bonded to the terminal of the linear or branched aliphatic hydrocarbon group, and a group in which the alicyclic hydrocarbon group is interposed in the middle of the linear or branched aliphatic hydrocarbon group. Examples of the linear or branched aliphatic hydrocarbon group include the same groups as those for the linear aliphatic hydrocarbon group or the branched aliphatic hydrocarbon group.

The alicyclic hydrocarbon group has preferably 3 to 20 carbon atoms and more preferably 3 to 12 carbon atoms.

The alicyclic hydrocarbon group may be monocyclic or polycyclic. The monocyclic alicyclic hydrocarbon group is preferably a group obtained by removing two hydrogen atoms from a monocycloalkane. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples thereof include adamantane, norbornane, isobornane, tricyclodecane, and tetracyclododecane.

The aromatic hydrocarbon group as the divalent hydrocarbon group represented by Va¹ is a hydrocarbon group having an aromatic ring.

The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 12 carbon atoms. Here, the number of carbon atoms in a substituent is not included in the number of carbon atoms.

Specific examples of the aromatic ring contained in the aromatic hydrocarbon group include aromatic hydrocarbon rings such as benzene, biphenyl, fluorene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom.

Specific examples of the aromatic hydrocarbon group include a group in which two hydrogen atoms have been removed from the above-described aromatic hydrocarbon ring (an arylene group); and a group in which one hydrogen atom of a group (an aryl group) formed by removing one hydrogen atom from the aromatic hydrocarbon ring has been substituted with an alkylene group (for example, a group formed by further removing one more hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

In Formula (a1-1), Ra¹ represents an acid dissociable group represented by General Formula (a1-r-1), (a1-r-2), or (a1-r-4).

In Formula (a1-2), the (nₐ₂+1)-valent hydrocarbon group as Wa¹ may be an aliphatic hydrocarbon group or an aromatic hydrocarbon group. The aliphatic hydrocarbon group indicates a hydrocarbon group that has no aromaticity and may be saturated or unsaturated. In general, it is preferable that the aliphatic hydrocarbon group is saturated. Examples of the aliphatic hydrocarbon group include a linear or branched aliphatic hydrocarbon group, an aliphatic hydrocarbon group having a ring in the structure thereof, and a group obtained by combining the linear or branched aliphatic hydrocarbon group and the aliphatic hydrocarbon group having a ring in the structure thereof.

The valency of nₐ₂+1 is preferably divalent, trivalent, or tetravalent and more preferably divalent or trivalent.

In Formula (a1-2), Ra² represents an acid dissociable group represented by General Formula (a1-r-1) or (a1-r-3).

Specific examples of the constitutional unit (a1) are shown below. In the formulae shown below, R^{α} represents a hydrogen atom, a methyl group, or a trifluoromethyl group.

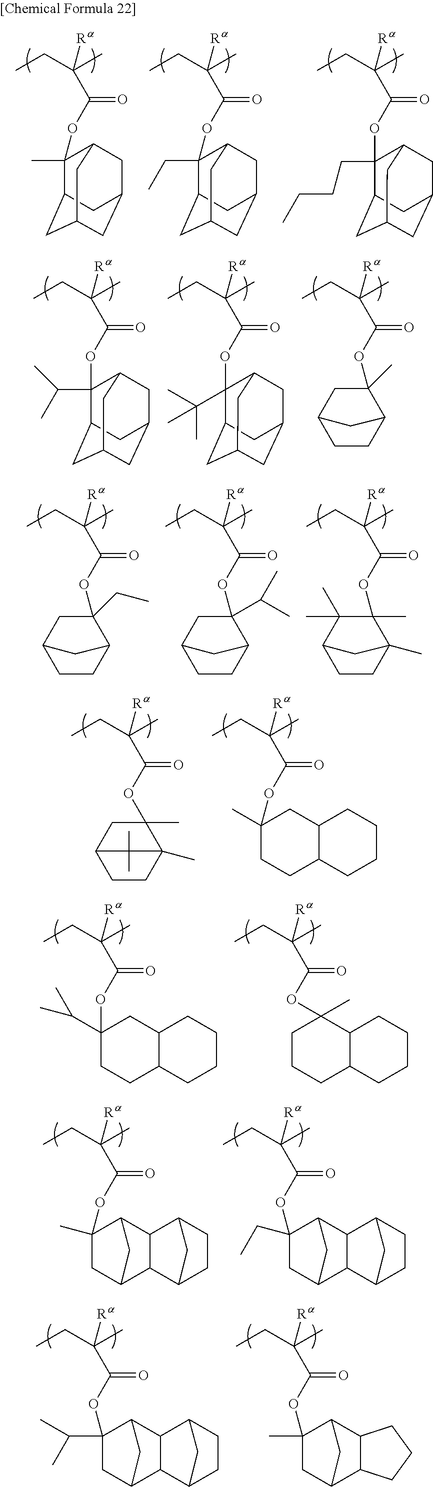

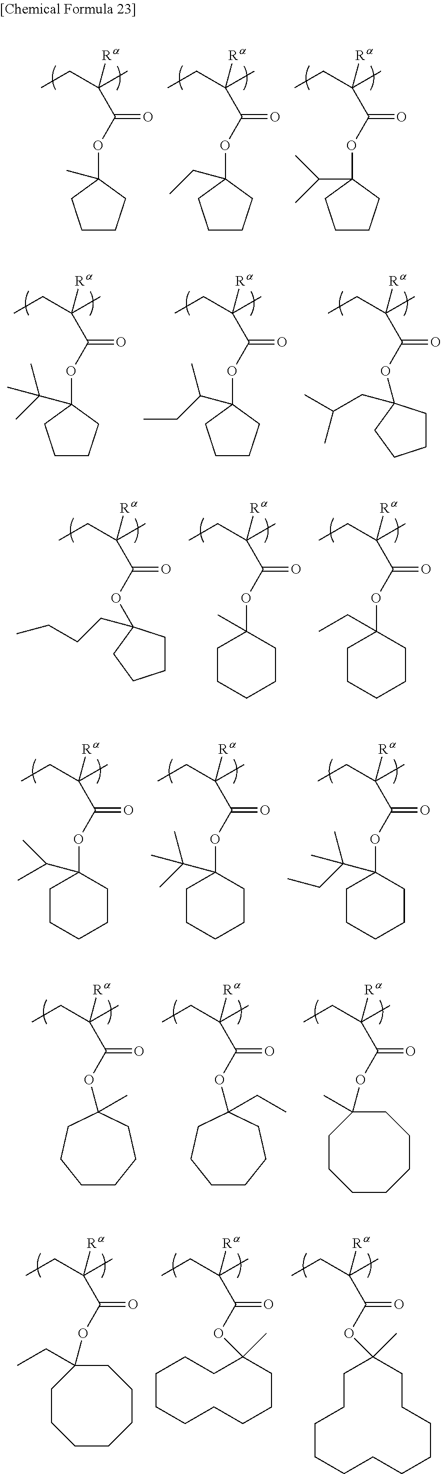

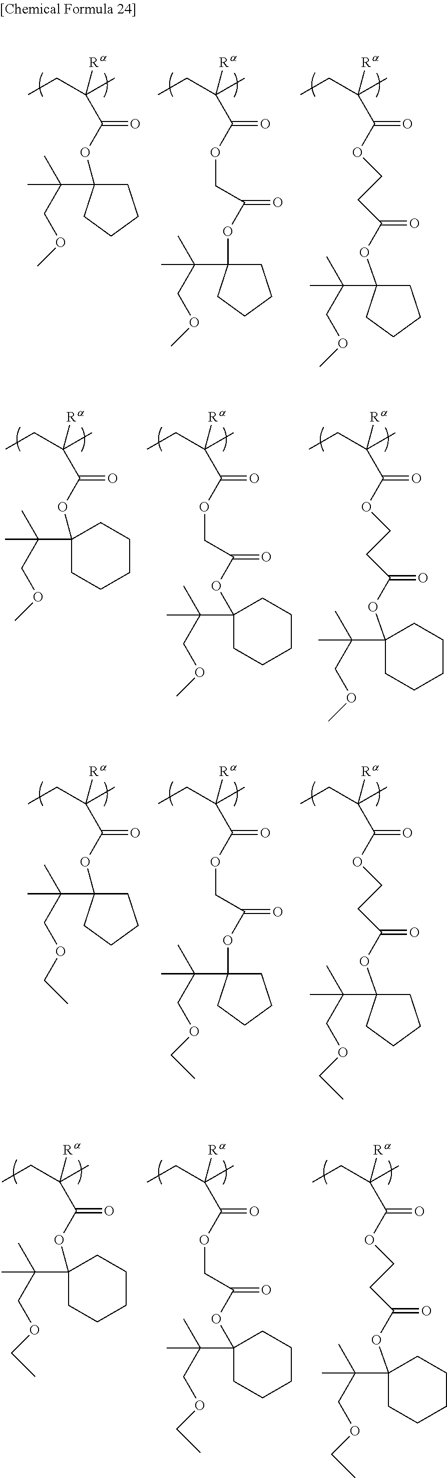

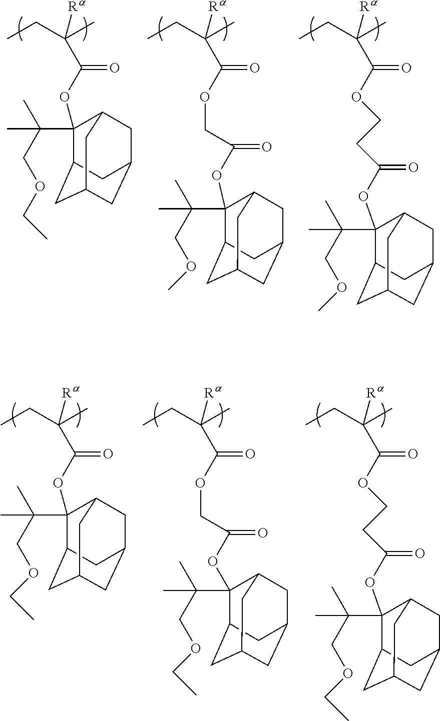

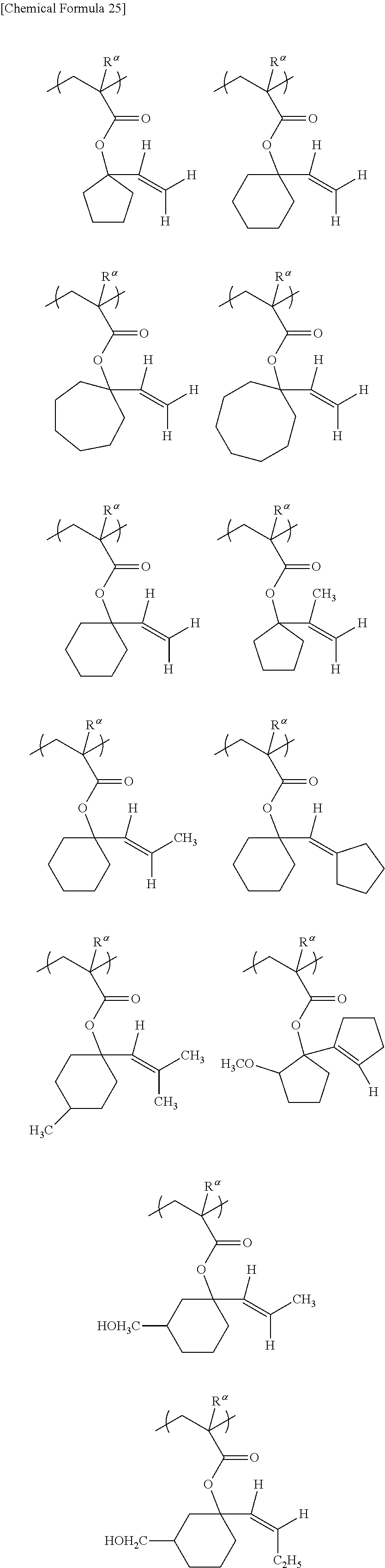

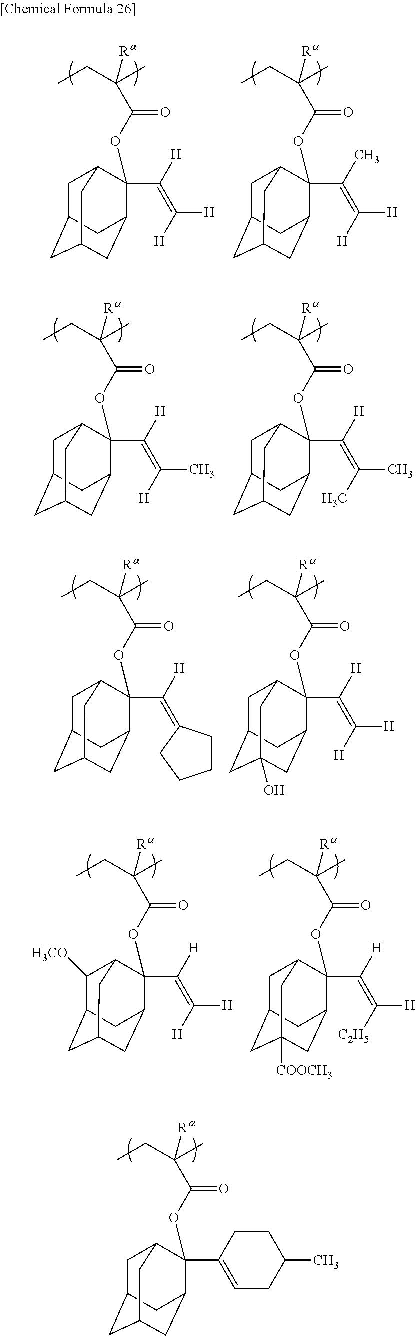

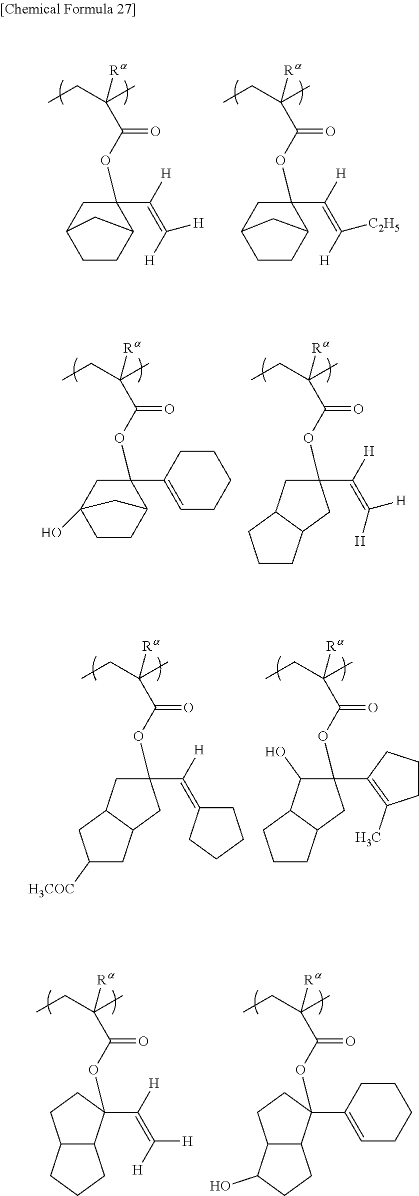

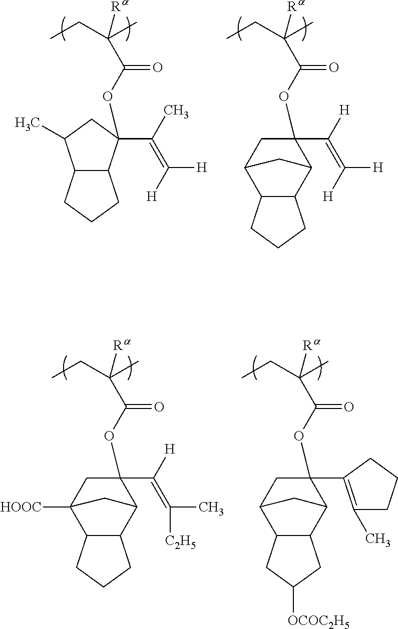

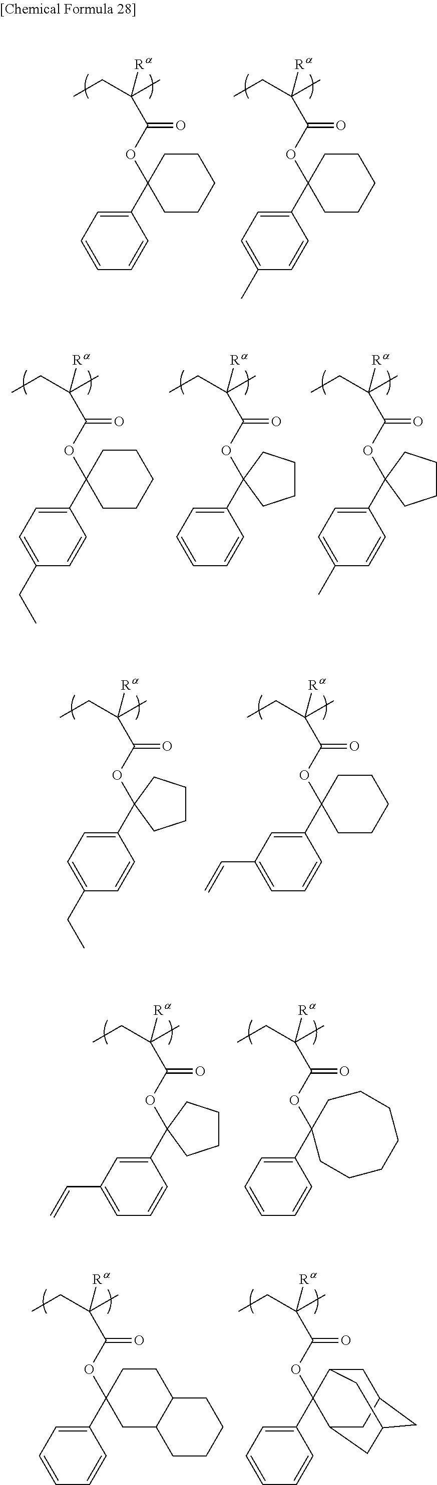

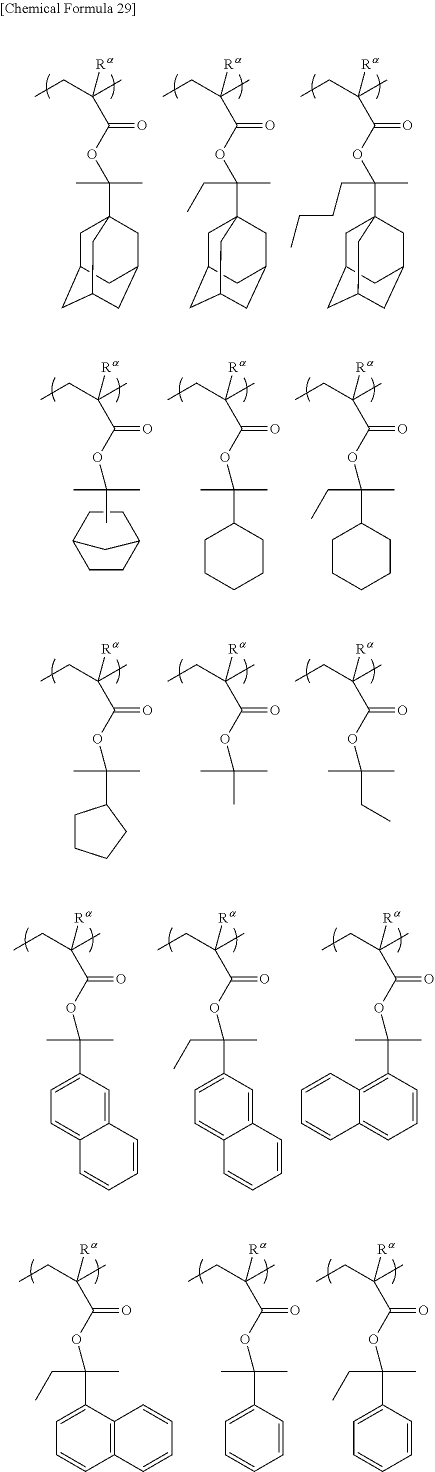

The constitutional unit (a1) included in the component (A1) may be used alone or two or more kinds thereof.

As the constitutional unit (a1), the constitutional unit represented by Formula (a1-1) is more preferable from the viewpoint that the lithography characteristics with electron beams or EUV are more likely to be enhanced.

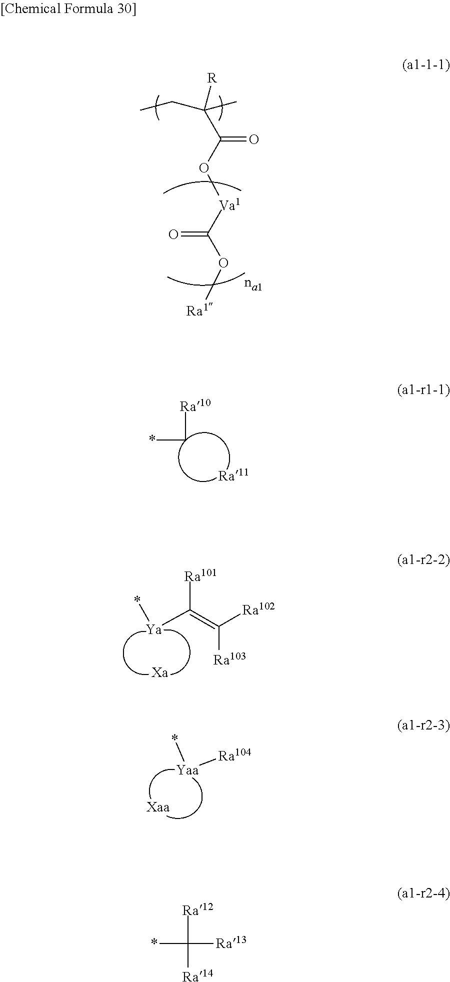

[In the formulae, Ra¹″ represents an acid dissociable group represented by General Formula (a1-r2-1), (a1-r2-2), (a1-r2-3), or (a1-r2-4)]. * represents a bonding site.]

In Formula (a1-1-1), R, Va¹, and nₐ₁ each have the same definition as that for R, Va¹, and nₐ₁ in Formula (a1-1).

The acid dissociable group represented by General Formula (a1-r2-1), (a1-r2-2), (a1-r2-3), or (a1-r2-4) is as described above. Among these, from the viewpoint of further increasing the reactivity for EB or EUV, an acid dissociable group in which the acid dissociable group is a cyclic group is preferably selected, an acid dissociable group represented by General Formula (a1-r2-1) or an acid dissociable group represented by General Formula (a1-r2-2) is more preferable, and an acid dissociable group represented by General Formula (a1-r2-1) is still more preferable.

In a case where the component (A1) has the constitutional unit (a1), the proportion of the constitutional unit (a1) in the component (A1) is preferably in a range of 5% to 95% by mole, more preferably in a range of 10% to 90% by mole, still more preferably in a range of 30% to 80% by mole, and particularly preferably in a range of 40% to 70% by mole with respect to the total amount (100% by mole) of all Constitutional units constituting the component (A1).

In a case where the proportion of the constitutional unit (a1) is set to be greater than or equal to the lower limits of the above-described preferable ranges, lithography characteristics such as the sensitivity, CDU, the resolution, and reduction of the roughness are improved. Further, in a case where the proportion of the constitutional unit (a1) is less than or equal to the upper limits of the above-described preferable ranges, the constitutional unit (a1) and other constitutional units can be balanced, and the lithography characteristics are improved.

In Regard to Constitutional Unit (a10):

The constitutional unit (a10) is a constitutional unit represented by General Formula (a10-1) (here, a constitutional unit corresponding to the constitutional unit (a1) is excluded).

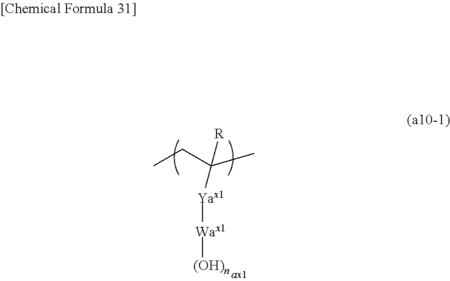

[In the formula, R represents a hydrogen atom, an alkyl group having 1 to 5 carbon atoms, or a halogenated alkyl group having 1 to 5 carbon atoms. Ya^{x1} represents a single bond or a divalent linking group. Wa^{x1} represents an aromatic hydrocarbon group which may have a substituent, nₐₓ₁ represents an integer of 1 or greater.]

In the Formula (a10-1), R represents a hydrogen atom, an alkyl group having 1 to 5 carbon atoms, or a halogenated alkyl group having 1 to 5 carbon atoms.

R represents preferably a hydrogen atom, an alkyl group having 1 to 5 carbon atoms, or a fluorinated alkyl group having 1 to 5 carbon atoms, and from the viewpoint of industrial availability, more preferably a hydrogen atom, a methyl group, or trifluoromethyl group, still more preferably a hydrogen atom or a methyl group, and particularly preferably a hydrogen atom.

In Formula (a10-1), Ya^{x1} represents a single bond or a divalent linking group.

In the chemical formula, the divalent linking group as Ya^{x1} is not particularly limited, and suitable examples thereof include a divalent hydrocarbon group which may have a substituent and a divalent linking group having a heteroatom.

Ya^{x1} represents preferably a single bond, an ester bond [—C(═O)—O— or —O—C(═O)—], an ether bond (—O—), a linear or branched alkylene group, or a combination thereof and more preferably a single bond or an ester bond [—C(═O))—O— or —O—C(═O)—].

In Formula (a10-1), Wa^{x1} represents an aromatic hydrocarbon group which may have a substituent.

Examples of the aromatic hydrocarbon group as Wa^{x1} include a group obtained by removing (nₐₓ₁+1) hydrogen atoms from an aromatic ring which may have a substituent. The aromatic ring is not particularly limited as long as the aromatic ring is a cyclic conjugated system having (4n+2)π electrons. The aromatic ring has preferably 5 to 30 carbon atoms, more preferably 5 to 20 carbon atoms, still more preferably 6 to 15 carbon atoms, and particularly preferably 6 to 12 carbon atoms. Specific examples of the aromatic ring include aromatic hydrocarbon rings such as benzene, naphthalene, anthracene, and phenanthrene; and an aromatic heterocyclic ring obtained by substituting some carbon atoms constituting the above-described aromatic hydrocarbon ring with a heteroatom. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Examples of the aromatic hydrocarbon group as Wa^{x1} also include a group obtained by removing (nₐₓ₁+1) hydrogen atoms from an aromatic compound including an aromatic ring (for example, biphenyl or fluorene) which may have two or more substituents.

Among these, Wa^{x1} represents preferably a group in which (nₐₓ₁+1) hydrogen atoms have been removed from benzene, naphthalene, anthracene, or biphenyl, more preferably a group in which (nₐₓ₁+1) hydrogen atoms have been removed from benzene or naphthalene, and still more preferably a group in which (nₐₓ₁+1) hydrogen atoms have been removed from benzene.

The aromatic hydrocarbon group as Wa^{x1} may have a substituent or may not have a substituent. Examples of substituents include an alkyl group, an alkoxy group, a halogen atom, and a halogenated alkyl group. Examples of the alkyl group, the alkoxy group, the halogen atom, and the halogenated alkyl group as the substituent include the same groups as those for the substituent of the cyclic alicyclic hydrocarbon group as Ya^{x1}. The substituent is preferably a linear or branched alkyl group having 1 to 5 carbon atoms, more preferably a linear or branched alkyl group having 1 to 3 carbon atoms, still more preferably an ethyl group or a methyl group, and particularly preferably a methyl group. It is preferable that the aromatic hydrocarbon group as Wa^{x1} has no substituent.

In Formula (a10)-1), nₐₓ₁ represents an integer of 1 or greater, preferably an integer of 1 to 10, more preferably an integer of 1 to 5, still more preferably 1, 2, or 3, and particularly preferably 1 or 2.

Specific examples of the constitutional unit (a10) represented by Formula (a10-1) are described below.

In the formulae shown below, R^{α} represents a hydrogen atom, a methyl group, or a trifluoromethyl group.

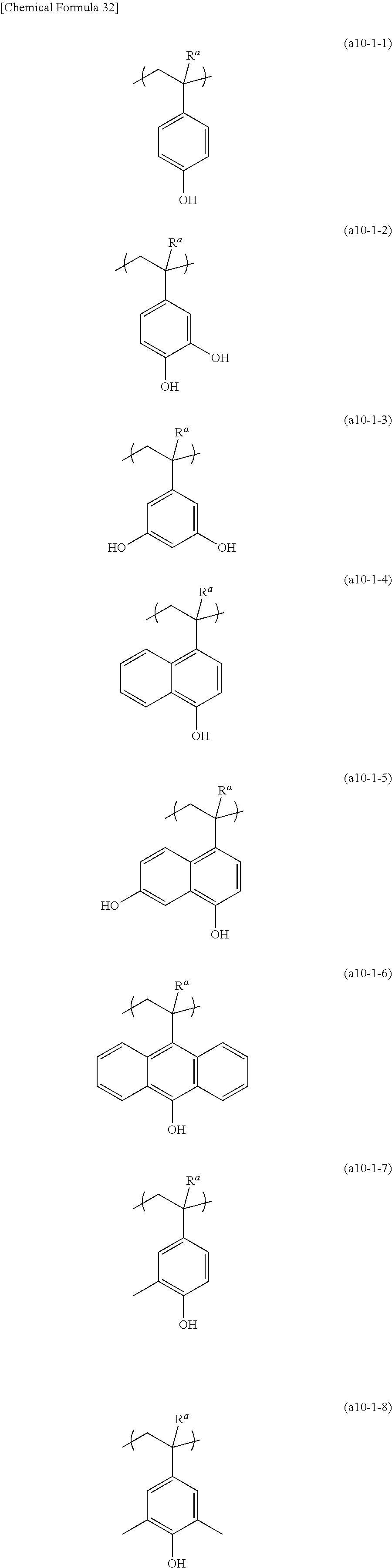

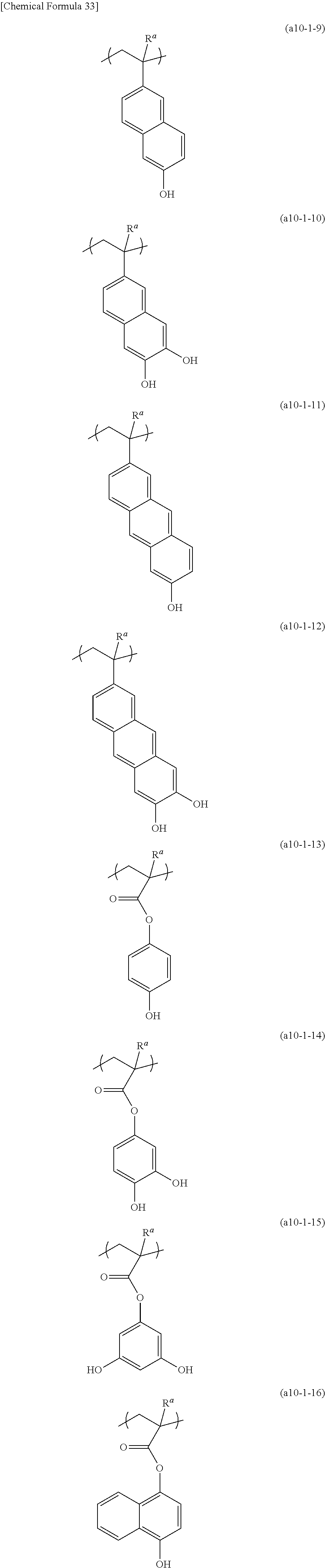

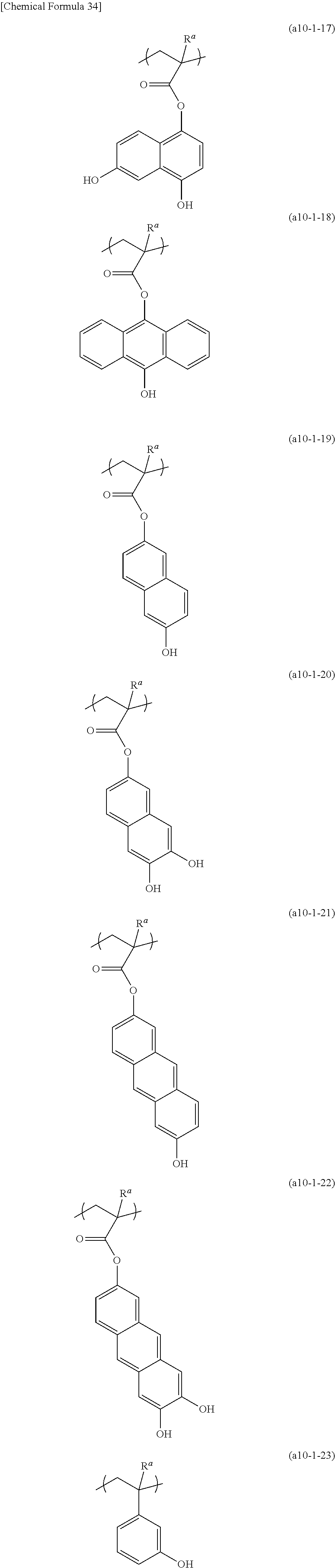

The constitutional unit (a10) included in the component (A1) may be used alone or two or more kinds thereof.

In a case where the component (A1) has the constitutional unit (a10), the proportion of the constitutional unit (a10) in the component (A1) is preferably in a range of 20% to 60% by mole, more preferably in a range of 25% to 55% by mole, still more preferably in a range of 30% to 50% by mole, and particularly preferably in a range of 35% to 45% by mole with respect to the total amount (100% by mole) of all constitutional units constituting the component (A1).

In a case where the proportion of the constitutional unit (a10) is set to be greater than or equal to the above-described lower limits, the sensitivity is likely to be enhanced. Meanwhile, in a case where the proportion thereof is set to be less than or equal to the above-described upper limits, the constitutional unit (a10) and other constitutional units are likely to be balanced.

### In Regard to Constitutional Unit (a2):

The component (A1) may further have a constitutional unit (a2) (here, a constitutional unit corresponding to the constitutional unit (a1) is excluded) containing a lactone-containing cyclic group.

In a case where the component (A1) is used to form a resist film, the lactone-containing cyclic group of the constitutional unit (a2) is effective for increasing the adhesiveness of the resist film to the substrate. Further, in a case where the component (A1) contains the constitutional unit (a2), the lithography characteristics and the like are improved due to the effects of appropriately adjusting the acid diffusion length, increasing the adhesiveness of the resist film to the substrate, and appropriately adjusting the solubility during the development.

The term “lactone-containing cyclic group” indicates a cyclic group that has a ring (lactone ring) containing —O—C(═O)— in the ring skeleton. In a case where the lactone ring is counted as the first ring and the group contains only the lactone ring, the group is referred to as a monocyclic group. Further, in a case where the group has other ring structures, the group is referred to as a polycyclic group regardless of the structures. The lactone-containing cyclic group may be a monocyclic group or a polycyclic group.

The lactone-containing cyclic group in the constitutional unit (a2) is not particularly limited, and an optional constitutional unit can be used. Specific examples thereof include groups each represented by General Formulae (a2-r-1) to (a2-r-7).

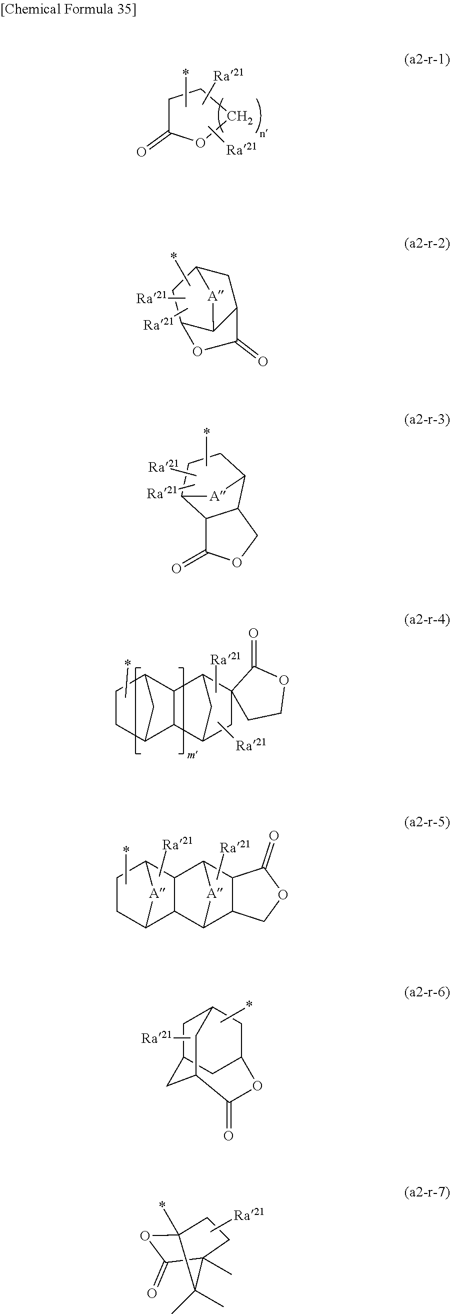

[In the formulae, Ra′²¹'s each independently represent a hydrogen atom, an alkyl group, an alkoxy group, a halogen atom, a halogenated alkyl group, a hydroxyl group, —COOR″c, —OC(═O)R″, a hydroxyalkyl group, or a cyano group, R″ represents a hydrogen atom, an alkyl group, or a lactone-containing cyclic group, A″ represents an alkylene group having 1 to 5 carbon atoms which may have an oxygen atom (—O—) or a sulfur atom (—S—), an oxygen atom, or a sulfur atom, n′ represents an integer of 0 to 2, and m′ represents 0 or 1. * represents a bonding site (the same applies hereinafter).]

In General Formulae (a2-r-1) to (a2-r-7), it is preferable that the alkyl group as Ra′²¹ is an alkyl group having 1 to 6 carbon atom. Further, it is preferable that the alkyl group is linear or branched. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or ethyl group is preferable, and a methyl group is particularly preferable.

It is preferable that the alkoxy group as Ra′²¹ is an alkoxy group having 1 to 6 carbon atoms. Further, it is preferable that the alkoxy group is linear or branched. Specific examples of the alkoxy groups include a group formed by linking the above-described alkyl group exemplified as the alkyl group represented by Ra′²¹ to an oxygen atom (—O—).

As the halogen atom as Ra′²¹, a fluorine atom is preferable.

Examples of the halogenated alkyl group as Ra′²¹ include groups in which some or all hydrogen atoms in the alkyl group as Ra′²¹ have been substituted with the halogen atoms. As the halogenated alkyl group, a fluorinated alkyl group is preferable, and a perfluoroalkyl group is particularly preferable.

In —COOR″ and —OC(═O) R″ as Ra′²¹, each R″ represents a hydrogen atom, an alkyl group, or a lactone-containing cyclic group.

The alkyl group as R″ may be linear, branched, or cyclic and has preferably 1 to 15 carbon atoms.

In a case where R″ represents a linear or branched alkyl group, an alkyl group having 1 to 10 carbon atoms is preferable, an alkyl group having 1 to 5 carbon atoms is more preferable, and a methyl group or an ethyl group is particularly preferable.

In a case where R″ represents a cyclic alkyl group, the number of carbon atoms thereof is preferably in a range of 3 to 15, more preferably in a range of 4 to 12, and most preferably in a range of 5 to 10. Specific examples thereof include groups in which one or more hydrogen atoms have been removed from a monocycloalkane, which may or may not be substituted with a fluorine atom or a fluorinated alkyl group; and groups in which one or more hydrogen atoms have been removed from a polycycloalkane such as bicycloalkane, tricycloalkane, or tetracycloalkane. More specific examples thereof include groups in which one or more hydrogen atoms have been removed from a monocycloalkane such as cyclopentane or cyclohexane; and groups in which one or more hydrogen atoms have been removed from a polycycloalkane such as adamantane, norbornane, isobornane, tricyclodecane, or tetracyclododecane.

Examples of the lactone-containing cyclic group as R″ include the same groups as those for the groups each represented by General Formulae (a2-r-1) to (a2-r-7).

As the hydroxyalkyl group as Ra′²¹, a hydroxyalkyl group having 1 to 6 carbon atoms is preferable, and specific examples thereof include a group in which at least one hydrogen atom in the alkyl group as Ra′²¹ has been substituted with a hydroxyl group.

Among the examples, it is preferable that Ra′²¹'s each independently represent a hydrogen atom or a cyano group.

In General Formulae (a2-r-2), (a2-r-3) and (a2-r-5), as the alkylene group having 1 to 5 carbon atoms as A″, a linear or branched alkylene group is preferable, and examples thereof include a methylene group, an ethylene group, an n-propylene group, and an isopropylene group. Specific examples of the alkylene groups that contain an oxygen atom or a sulfur atom include a group obtained by interposing —O— or —S— in the terminal of the alkylene group or between the carbon atoms of the alkylene group, and examples thereof include —O—CH₂—, —CH₂—O—CH₂, —S—CH₂—, and —CH₂S—CH₂—. A″ represents preferably an alkylene group having 1 to 5 carbon atoms or —O—, more preferably an alkylene group having 1 to 5 carbon atoms, and most preferably a methylene group.

Specific examples of the groups each represented by General Formulae (a2-r-1) to (a2-r-7) are shown below.

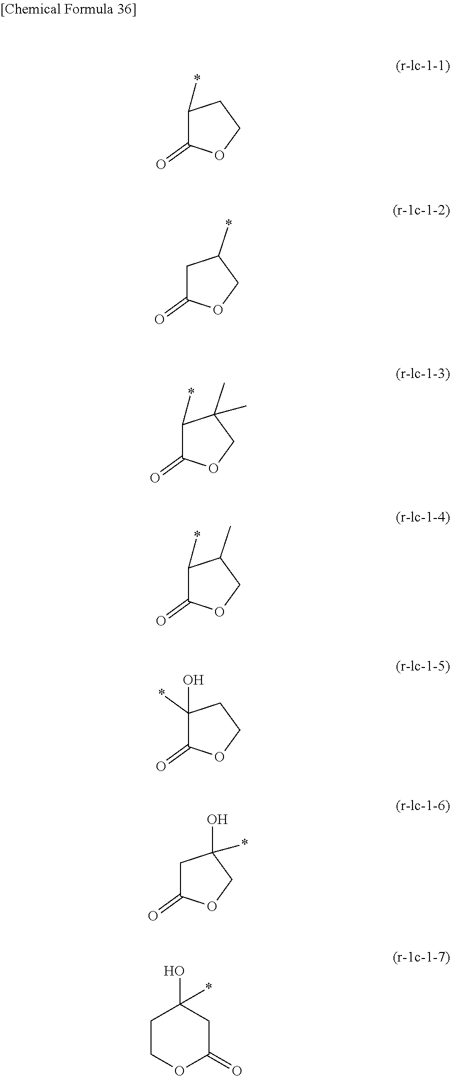

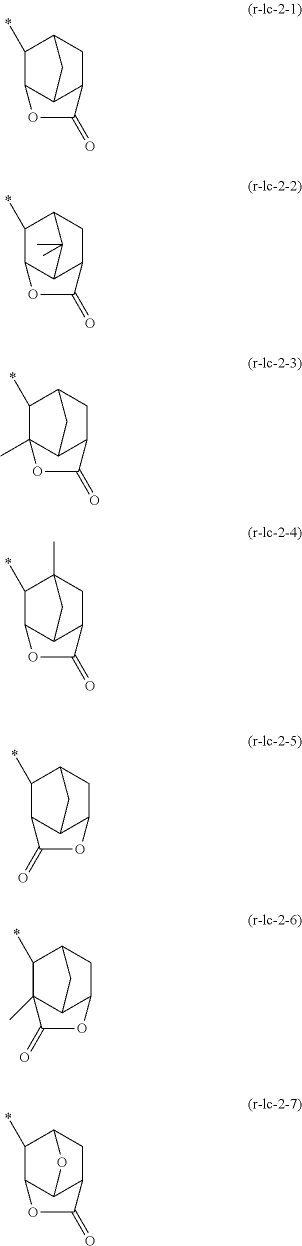

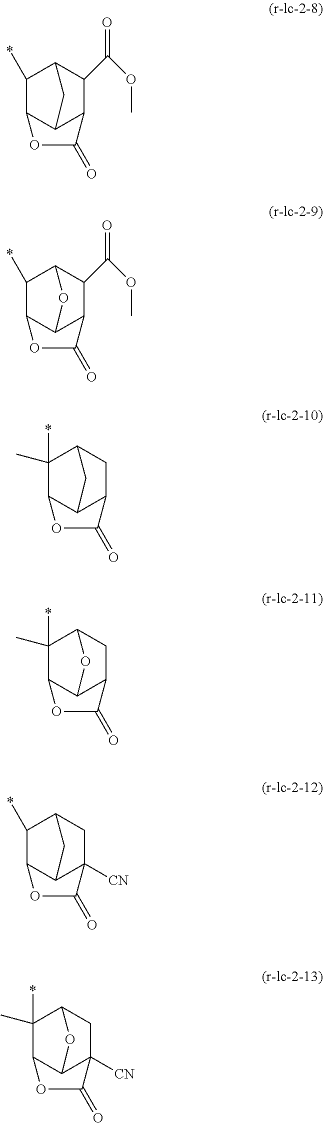

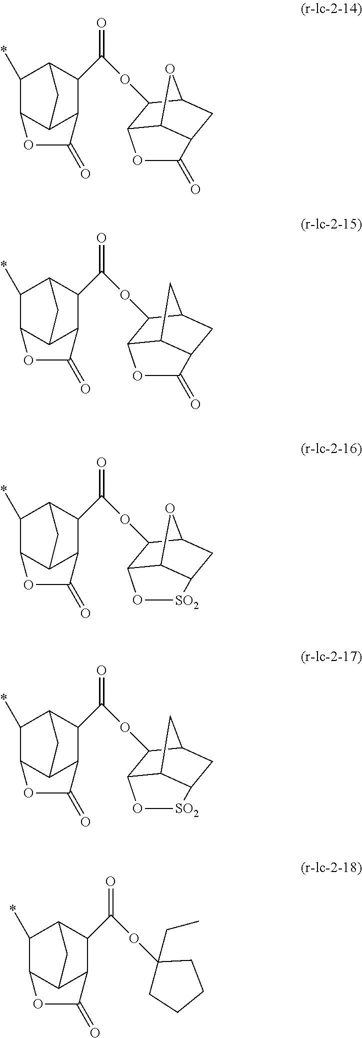

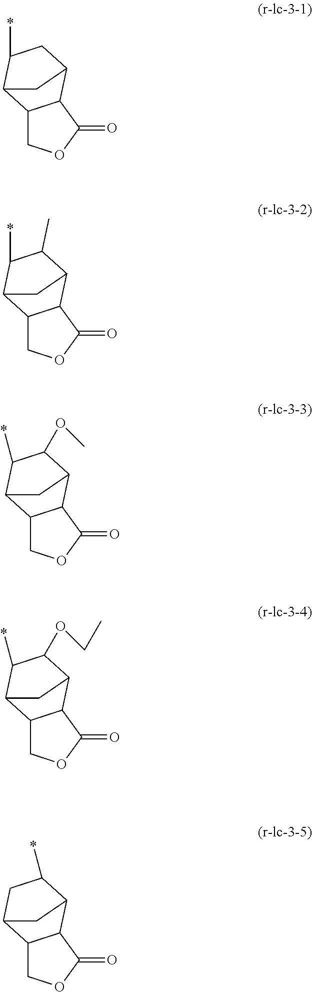

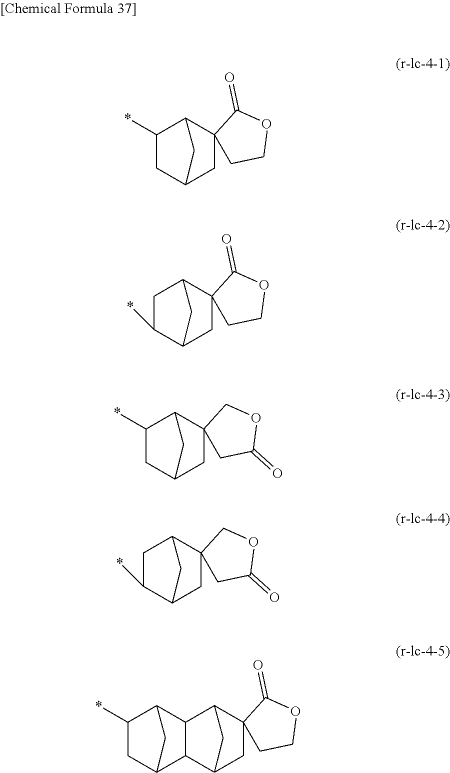

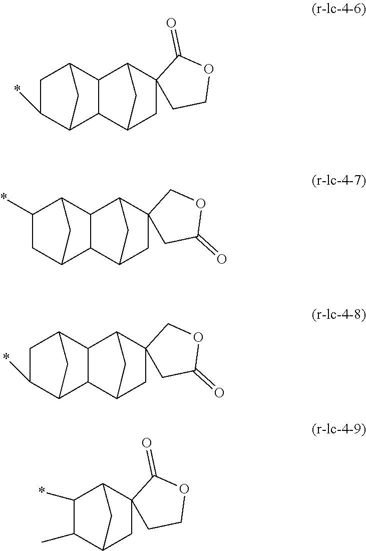

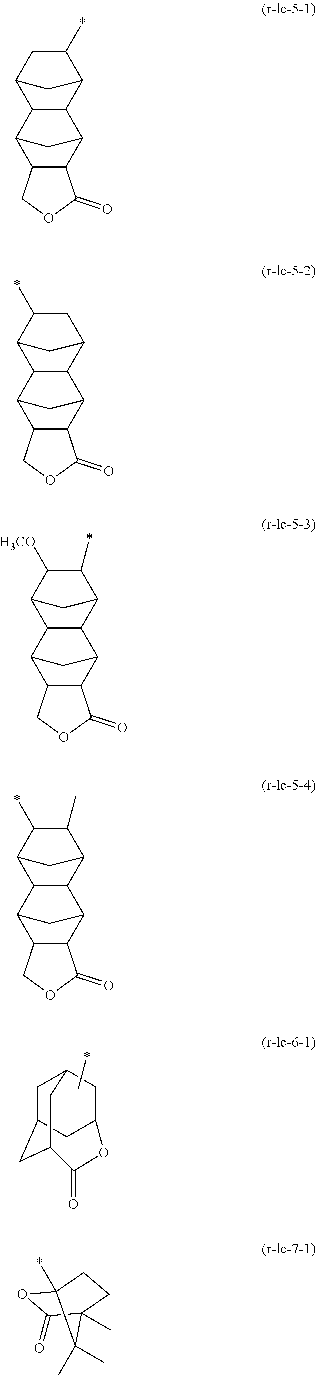

As the constitutional unit (a2), a constitutional unit derived from acrylic acid ester in which the hydrogen atom bonded to the carbon atom at the α-position may be substituted with a substituent is preferable.

It is preferable that such a constitutional unit (a2) is a constitutional unit represented by General Formula (a2-1).

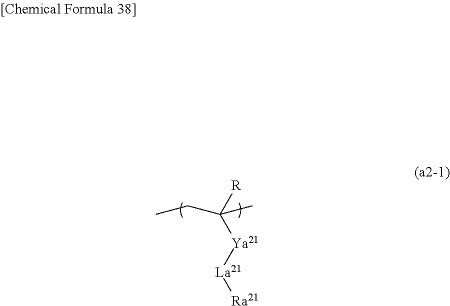

[In the formula, R represents a hydrogen atom, an alkyl group having 1 to 5 carbon atoms, or a halogenated alkyl group having 1 to 5 carbon atoms, Ya²¹ represents a single bond or a divalent linking group. La²¹ represents —O—, —COO—, —CON(R′)—, —OCO—, —CONHCO—, or —CONHCS—, and R′ represents a hydrogen atom or a methyl group. In a case where La²¹ represents —O—, Ya²¹ does not represent —CO—. Ra²¹ represents a lactone-containing cyclic group.]

In Formula (a2-1), R has the same definition as described above. R represents preferably a hydrogen atom, an alkyl group having 1 to 5 carbon atoms, or a fluorinated alkyl group having 1 to 5 carbon atoms and particularly preferably a hydrogen atom or a methyl group from the viewpoint of the industrial availability.

In Formula (a2-1), the divalent linking group as Ya²¹ is not particularly limited, and suitable examples thereof include a divalent hydrocarbon group which may have a substituent and a divalent linking group having heteroatoms.

It is preferable that Ya²¹ represents a single bond, an ester bond [—C(═O)—O—], an ether bond (—O—), a linear or branched alkylene group, or a combination thereof.

In Formula (a2-1), it is preferable that Ya²¹ represents a single bond and La²¹ represents —COO— or —OCO—.

In Formula (a2-1), Ra²¹ represents a lactone-containing cyclic group.

Suitable examples of the lactone-containing cyclic group as Ra²¹ include groups each represented by General Formulae (a2-r-1) to (a2-r-7).

The constitutional unit (a2) included in the component (A1) may be used alone or two or more kinds thereof.

In a case where the component (A1) has the constitutional unit (a2), the proportion of the constitutional unit (a2) is preferably in a range of 1% to 20% by mole, more preferably in a range of 1% to 15% by mole, and still more preferably in a range of 1% to 10% by mole with respect to the total amount (100% by mole) of all constitutional units constituting the component (A1).

In a case where the proportion of the constitutional unit (a2) is set to be greater than or equal to the lower limits of the above-described preferable ranges, the effect to be obtained by allowing the component (A1) to have the constitutional unit (a2) is sufficiently obtained by the above-described effects. Further, in a case where the proportion thereof is set to be less than or equal to the upper limits of the above-described preferable ranges, the constitutional unit (a2) and other constitutional units can be balanced, and the lithography characteristics are improved.

### In Regard to Constitutional Unit (a8):

The constitutional unit (a8) is a constitutional unit derived from a compound represented by General Formula (a8-1).

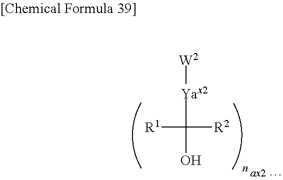

[In the formula, W² represents a polymerizable group-containing group. Ya^{x2} represents a single bond or an (nₐₓ₂+1)-valent linking group. Ya^{x2} and W² may form a condensed ring. R¹ represents a fluorinated alkyl group having 1 to 12 carbon atoms. R² represents an organic group having 1 to 12 carbon atoms, which may have a fluorine atom, or a hydrogen atom. R² and Ya^{x2} may be bonded to each other to form a ring structure by being bonded to each other. nₐₓ₂ represents an integer of 1 to 3.]

The term “polymerizable group” in the polymerizable group-containing group as W² is a group that enables a compound having the polymerizable group to be polymerized by radical polymerization or the like, and refers to a group containing a multiple bond between carbon atoms, such as an ethylenic double bond.

The polymerizable group-containing group may be a group formed of only a polymerizable group or a group formed of a polymerizable group and a group other than the polymerizable group. Examples of the group other than the polymerizable group include a divalent hydrocarbon group which may have a substituent and a divalent linking group containing a heteroatom.

Suitable examples of the polymerizable group-containing group include a group represented by a chemical formula: C(R^{X11}) (R^{X12})═C(R^{X13})-Ya^{x0}-.

In the chemical formula, R^{X11}, R^{X12}, and R^{X13} each represent a hydrogen atom, an alkyl group having 1 to 5 carbon atoms, or a halogenated alkyl group having 1 to 5 carbon atoms, and Ya^{x0} represents a single bond or a divalent linking group.

Examples of the condensed ring formed by Ya^{x2} and W² include a condensed ring formed by a polymerizable group of the W² moiety and by Ya^{x2} and a condensed ring formed by a group other than the polymerizable group of the W² moiety and by Ya^{x2}.

The condensed ring formed by Ya^{x2} and W² may have a substituent.

Specific examples of the constitutional unit (a8) are shown below.

In the following formulae. R^{α} represents a hydrogen atom, a methyl group, or a trifluoromethyl group.

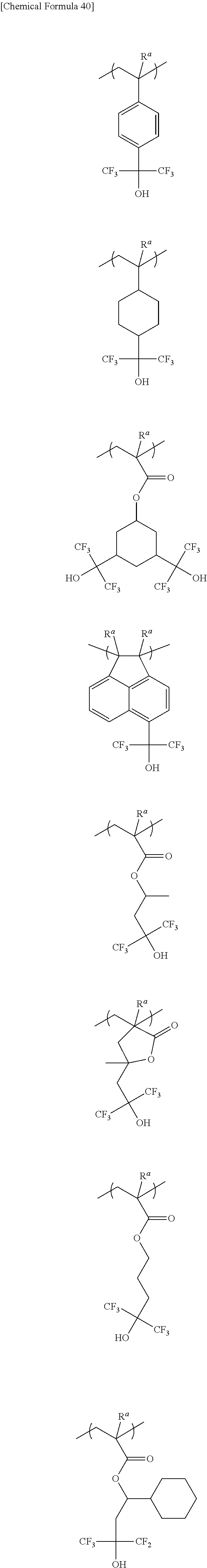

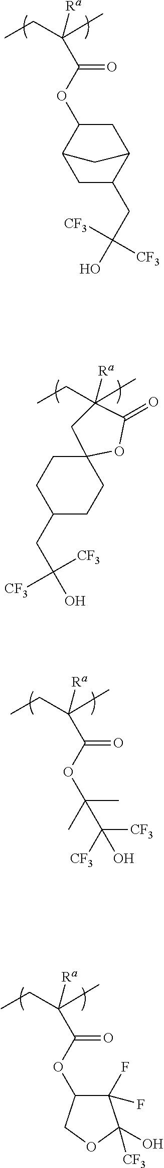

Among the examples, the constitutional unit (a8) is preferably at least one selected from the group consisting of constitutional units each represented by Chemical Formulae (a8-1-01) to (a8-1-04), (a8-1-06), (a8-1-08), (a8-1-09), and (a8-1-10) and more preferably at least one selected from the group consisting of constitutional units each represented by Chemical Formulae (a8-1-01) to (a8-1-04) and (a8-1-09).

The constitutional unit (a8) contained in the component (A1) may be used alone or two or more kinds thereof.

The proportion of the constitutional unit (a8) in the component (A1) is preferably 50% by mole or less and more preferably in a range of 0% to 30% by mole with respect to the total amount (100% by mole) of all constitutional units constituting the component (A1).

The component (A1) contained in the resist composition may be used alone or in combination of two or more kinds thereof.

In the resist composition of the present embodiment, preferred examples of the component (A1) include a polymer compound having a repeating structure of the constitutional unit (a1).

Among the examples, suitable examples of the component (A1) include a polymer compound having a repeating structure of the constitutional unit (a1) and the constitutional unit (a10).

In the polymer compound having a repeating structure of the constitutional unit (a1) and the constitutional unit (a10), the proportion of the constitutional unit (a1) is preferably in a range of 40% to 80% by mole, more preferably in a range of 45% to 75% by mole, still more preferably in a range of 50% to 70% by mole, and particularly preferably in a range of 55% to 65% by mole with respect to the total amount (100% by mole) of all constitutional units constituting the polymer compound.

In addition, the proportion of the constitutional unit (a10) in the polymer compound is preferably in a range of 20% to 60% by mole, more preferably in a range of 25% to 55% by mole, still more preferably in a range of 30% to 50% by mole, and particularly preferably in a range of 35% to 45% by mole with respect to the total amount (100% by mole) of all constitutional units constituting the polymer compound.

Such a component (A1) can be produced by dissolving a monomer, from which each constitutional unit is derived, in a polymerization solvent and adding a radical polymerization initiator such as azobisisobutyronitrile (AIBN) or dimethyl azobisisobutyrate (for example, V-601) to the solution so that the polymerization is carried out.

Alternatively, the component (A1) can be produced by dissolving, in a polymerization solvent, a monomer from which the constitutional unit (a1) is derived and, as necessary, a monomer from which a constitutional unit (for example, the constitutional unit (a10)) other than the constitutional unit (a1) is derived, adding thereto a radical polymerization initiator as described above to carry out polymerization, and then carrying out a deprotection reaction.

Further, a —C(CF₃)₂—OH group may be introduced into the terminal of the component (A1) during the polymerization using a chain transfer agent such as HS—CH₂—CH₂—CH₂—C(CF₃)₂—OH in combination. As described above, a copolymer into which a hydroxyalkyl group, formed by substitution of some hydrogen atoms in the alkyl group with fluorine atoms, has been introduced is effective for reducing development defects and reducing line edge roughness (LER: uneven irregularities of a line side wall).

The weight-average molecular weight (Mw) (in terms of polystyrene according to gel permeation chromatography (GPC)) of the component (A1) is not particularly limited, but is preferably in a range of 1000 to 50000, more preferably in a range of 2000 to 30000, and still more preferably in a range of 3000 to 20000.

In a case where the Mw of the component (A1) is less than or equal to the upper limits of the above-described preferable ranges, the resist composition exhibits a satisfactory solubility in a resist solvent for a resist enough to be used as a resist. On the contrary, in a case where the Mw of the component (A1) is greater than or equal to the lower limits of the above-described preferable ranges, the dry etching resistance and the cross-sectional shape of the resist pattern are excellent.

Further, the dispersity (Mw/Mn) of the component (A1) is not particularly limited, but is preferably in a range of 1.0 to 4.0, more preferably in a range of 1.0 to 3.0, and particularly preferably in a range of 1.0 to 2.0. Further, Mn represents the number average molecular weight.

### In Regard to Component (A2)

In the resist composition of the present embodiment, a base material component (hereinafter, also referred to as “component (A2)”) which does not correspond to the component (A1) and whose solubility in a developing solution is changed by the action of an acid may be used in combination as the component (A).

The component (A2) is not particularly limited and may be optionally selected from a plurality of components of the related art which have been known as base material components for a chemically amplified resist composition and used.

As the component (A2), a polymer compound or a low-molecular-weight compound may be used alone or in combination of two or more kinds thereof.

The proportion of the component (A1) in the component (A) is preferably 25% by mass or greater, more preferably 50% by mass or greater, and still more preferably 75% by mass or greater, and may be 100% by mass with respect to the total mass of the component (A). In a case where the proportion thereof is 25% by mass or greater, a resist pattern having various excellent lithography characteristics such as high sensitivity, resolution, and roughness reduction is likely to be formed.

In the resist composition of the present embodiment, the content of the component (A) may be adjusted according to the thickness of the resist film intended to be formed.

### <Component (B): Acid Generator Component>

The resist composition according to the present embodiment further contains, in addition to the component (A), an acid generator component (component (B)) that generates an acid upon light exposure.

In the present embodiment, the component (B) contains a compound (B0) (hereinafter, also referred to as “component (B0)”) represented by General Formula (b0).

### In Regard to Component (B0)

The component (B0) is a compound represented by General Formula (b0).

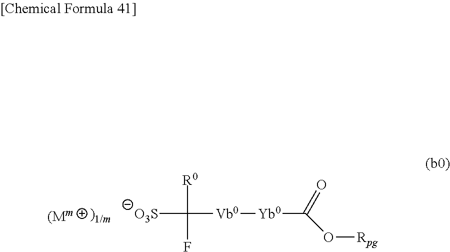

[In Formula (b0), M^{m+} represents an m-valent organic cation. m represents an integer of 1 or greater. R⁰ represents a fluorinated alkyl group having 1 to 5 carbon atoms or a fluorine atom. Vb⁰ represents an alkylene group, a fluorinated alkylene group, or a single bond. Yb⁰ represents a divalent linking group. Rpg represents an acid dissociable group represented by General Formula (a0-pg).]

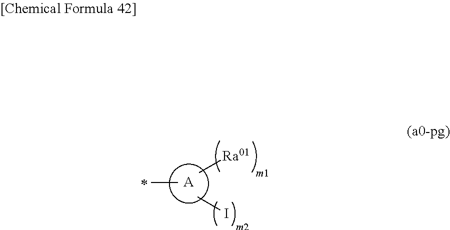

[In Formula (a0-pg), A represents a cyclic alkene having only one carbon-carbon unsaturated bond in a ring skeleton. Ra⁰¹ represents a monovalent hydrocarbon group having 1 to 20 carbon atoms. I represents an iodine atom. m1 represents an integer of 0 to 20. m2 represents an integer of 1 to 20. Here, m1+m2≤(a total number of carbon atoms constituting the ring skeleton of the cyclic alkene as A-1) is satisfied. In a case where m1 represents an integer of 2 or greater, a plurality of Ra⁰¹'s may be the same as or different from each other. * represents a bonding site that is bonded to —O— (oxy group) in —Yb⁰—CO—O—.]

### [Cation Moiety of Component (B0)]

In Formula (b0), M^{m+} represents an m-valent organic cation. m represents an integer of 1 or greater.

As the organic cation represented by M^{m+}, an onium cation is preferable, and among the examples, a sulfonium cation or an iodonium cation is more preferable.

Examples of the organic cation as M^{m+} include organic cations each represented by General Formulae (ca-1) to (ca-3).

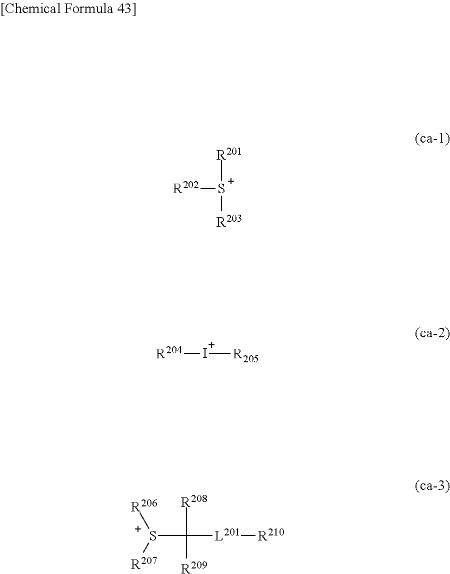

[In the formulae, R²⁰¹ to R²⁰⁷ each independently represent an aryl group, an alkyl group, or an alkenyl group, each of which may have a substituent. R²⁰¹ to R²⁰³, and R²⁰⁶ and R²⁰⁷ may be bonded to each other to form a ring together with the sulfur atoms in the formulae. R²⁰⁸ and R²⁰⁹ each independently represent a hydrogen atom or an alkyl group having 1 to 5 carbon atoms. R²¹⁰ represents an aryl group which may have a substituent, an alkyl group which may have a substituent, an alkenyl group which may have a substituent, or a —SO₂-containing cyclic group which may have a substituent. L²⁰¹ represents —C(═O)— or —C(═O)—O—.]

In Formulae (ca-1) to (ca-3), examples of the aryl group as R²⁰¹ to R²⁰⁷ include an unsubstituted aryl group having 6 to 20 carbon atoms, and a phenyl group or a naphthyl group is preferable.

The alkyl group as R²⁰¹ to R²⁰⁷ is preferably a chain-like or cyclic alkyl group which has 1 to 30 carbon atoms.

The alkenyl group as R²⁰¹ to R²⁰⁷ preferably has 2 to 10 carbon atoms.

Examples of the substituent which may be contained in R²⁰¹ to R²⁰⁷ and R²¹⁰ include an alkyl group, a halogen atom, a halogenated alkyl group, a carbonyl group, a cyano group, an amino group, an aryl group, and groups each represented by General Formulae (ca-r-1) to (ca-r-7).

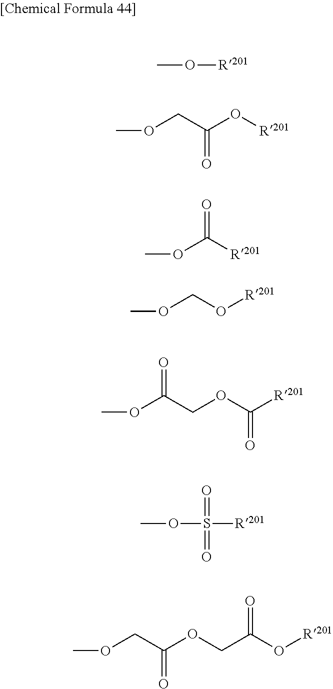

[In the formulae, R′²⁰¹'s each independently represents a hydrogen atom, a cyclic group which may have a substituent, a chain-like alkyl group which may have a substituent, or a chain-like alkenyl group which may have a substituent.]

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. Further, the aliphatic hydrocarbon group may be saturated or unsaturated. In general, it is preferable that the aliphatic hydrocarbon group is saturated.

The aromatic hydrocarbon group as R′²⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring contained in the aromatic hydrocarbon group as R′²⁰¹ include benzene, fluorene, naphthalene, anthracene, phenanthrene, biphenyl, or an aromatic heterocyclic ring in which some carbon atoms constituting any of these aromatic rings have been substituted with heteroatoms. Examples of the heteroatom in the aromatic heterocyclic rings include an oxygen atom, a sulfur atom, and a nitrogen atom.

Specific examples of the aromatic hydrocarbon group as R′²⁰¹ include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R′²⁰¹ include an aliphatic hydrocarbon group having a ring in the structure thereof.

Examples of the aliphatic hydrocarbon group having a ring in the structure thereof include an alicyclic hydrocarbon group (group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclodecane, or tetracyclododecane; and a polycycloalkane having a condensed ring polycyclic skeleton such as a cyclic group having a steroid skeleton are preferable as the polycycloalkane.

Among these examples, as the cyclic aliphatic hydrocarbon group as R′²⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is particularly preferable, and an adamantyl group is most preferable.

The linear or branched aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and particularly preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂; alkyltrimethylene groups such as —CH(CH₃)CH₂CH₂—, and —CH₂CH(CH₃)CH₂—; and alkyltetramethylene groups such as —CH(CH₃)CH₂CH₂CH₂—, and —CH₂CH(CH₃)CH₂CH₂— are exemplary examples. As the alkyl group in the alkylalkylene group, a linear alkyl group having 1 to 5 carbon atoms is preferable.

Further, the cyclic hydrocarbon group as R′²⁰¹ may have a heteroatom such as a heterocyclic ring. Specific examples thereof include lactone-containing cyclic groups each represented by General Formulae (a2-r-1) to (a2-r-7), —SO₂-containing cyclic groups each represented by General Formulae (b5-r-1) to (b5-r-4), and other heterocyclic groups each represented by Chemical Formulae (r-hr-1) to (r-hr-16).

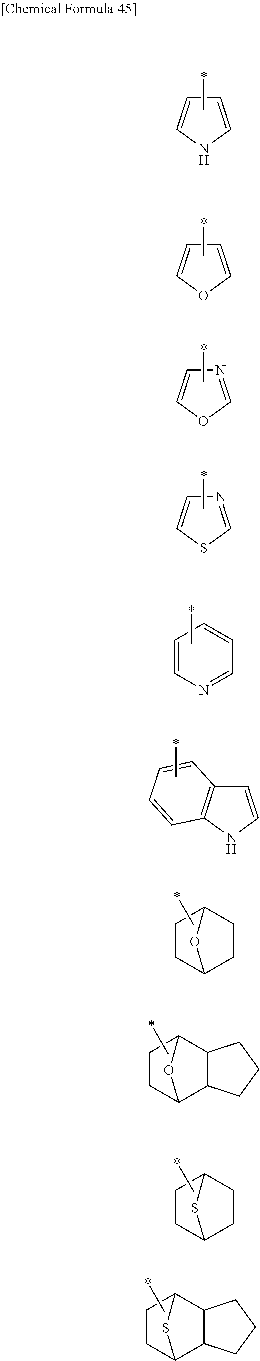

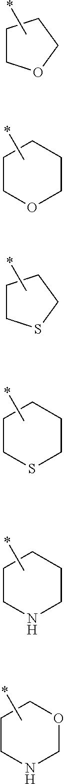

Examples of the substituent for the cyclic group as R′²⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

As the halogen atom as a substituent, a fluorine atom is preferable.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group have been substituted with the above-described halogen atoms.

The carbonyl group as the substituent is a group that substitutes a methylene group (—CH₂—) constituting the cyclic hydrocarbon group.

Chain-Like Alkyl Group which May have Substituent:

The chain-like alkyl group as R′²⁰¹ may be linear or branched.

The linear alkyl group has preferably 1 to 20 carbon atoms, more preferably 1 to carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R′²⁰¹ may be linear or branched, and has preferably 2 to 10 carbon atoms, more preferably 2 to 5 carbon atoms, still more preferably 2 to 4 carbon atoms, and particularly preferably 3 carbon atoms. Examples of the linear alkenyl group include a vinyl group, a propenyl group (allyl group), and a butenyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R′²⁰¹ include an alkoxy group, a halogen atom, a halogenated alkyl group, a hydroxyl group, a carbonyl group, a nitro group, an amino group, and a cyclic group as R′²⁰¹.

Examples of the cyclic group which may have a substituent, the chain-like alkyl group which may have a substituent, and the chain-like alkenyl group which may have a substituent as R′²⁰¹ include those for the acid dissociable group represented by Formula (a1-r-2) which are the exemplary examples of the cyclic group which may have a substituent and the chain-like alkyl group which may have a substituent, in addition to those described above.

Among the examples, R′²⁰¹ represents preferably a cyclic group which may have a substituent and more preferably a cyclic hydrocarbon group which may have a substituent. More specific preferred examples thereof include a phenyl group, a naphthyl group, a group in which one or more hydrogen atoms have been removed from a polycycloalkane, a lactone-containing cyclic group represented by any of General Formulae (a2-r-1) to (a2-r-7), and a —SO₂-containing cyclic group represented by any of General Formulae (b5-r-1) to (b5-r-4).

In General Formulae (ca-1) to (ca-3), in a case where R²⁰¹ to R³⁰³ and R²⁰⁶ and R²⁰⁷ are bonded to each other to form a ring with a sulfur atom in the formula, these groups may be bonded to each other via a heteroatom such as a sulfur atom, an oxygen atom, or a nitrogen atom, or a functional group such as a carbonyl group, —SO—, —SO₂—, —SO₃—, —COO—, —CONH—, or —N(R_{N})— (here, R_{N} represents an alkyl group having 1 to 5 carbon atoms). As a ring to be formed, a ring containing the sulfur atom in the formula in the ring skeleton thereof is preferably a 3- to 10-membered ring and particularly preferably a 5- to 7-membered ring containing the sulfur atom. Specific examples of the ring to be formed include a thiophene ring, a thiazole ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

R²⁰⁸ and R²⁰⁹ each independently represent a hydrogen atom or an alkyl group having 1 to 5 carbon atoms and preferably a hydrogen atom or an alkyl group having 1 to 3 carbon atoms. In a case where R²⁰⁸ and R²⁰⁹ each represent an alkyl group, R²⁰⁸ and R²⁰⁹ may be bonded to each other to form a ring.

R²¹⁰ represents an aryl group which may have a substituent, an alkyl group which may have a substituent, an alkenyl group which may have a substituent, or a —SO₂— containing cyclic group which may have a substituent.

Examples of the aryl group as R²¹⁰ include an unsubstituted aryl group having 6 to 20 carbon atoms, and a phenyl group or a naphthyl group is preferable.

As the alkyl group as R²¹⁰, a chain-like or cyclic alkyl group having 1 to 30 carbon atoms is preferable.

The alkenyl group as R²¹⁰ preferably has 2 to 10 carbon atoms.

The —SO₂-containing cyclic group which may have a substituent as R²¹⁰ is preferably “—SO₂-containing polycyclic group” and more preferably a group represented by General Formula (b5-r-1).

Specific examples of the suitable cation represented by Formula (ca-1) include cations each represented by the following chemical formulae.

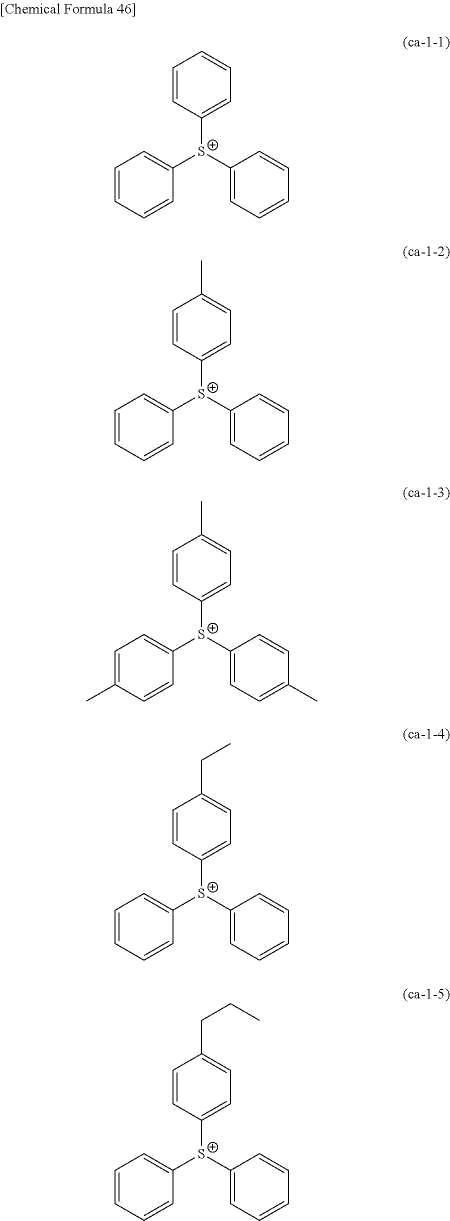

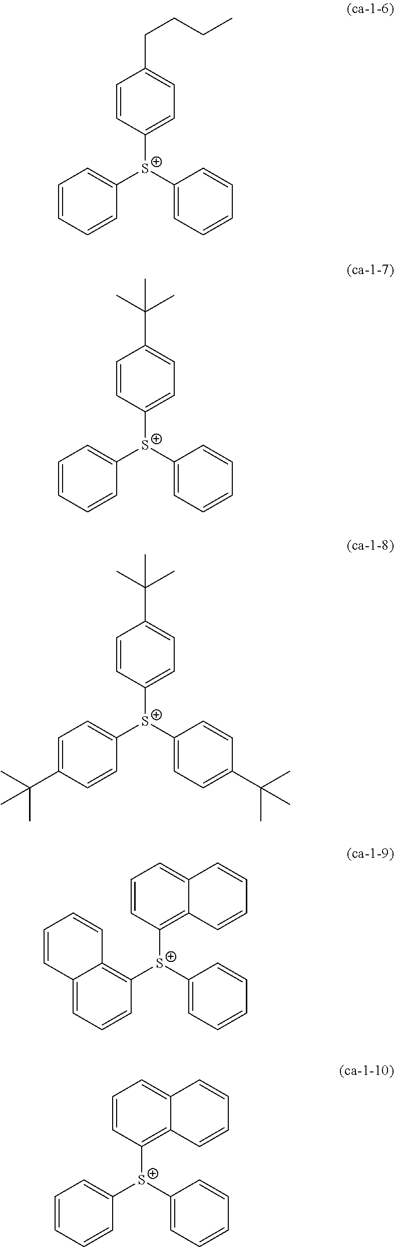 [SMILES: CCCCc1ccc([S+](c2ccccc2)c2ccccc2)cc1 ; CC(C)(C)c1ccc([S+](c2ccccc2)c2ccccc2)cc1 ; CC(C)(C)c1ccc([S+](c2ccc(C(C)(C)C)cc2)c2ccc(C(C)(C)C)cc2)cc1 ; c1ccc([S+](c2cccc3ccccc23)c2cccc3ccccc23)cc1 ; c1ccc([S+](c2ccccc2)c2cccc3ccccc23)cc1]

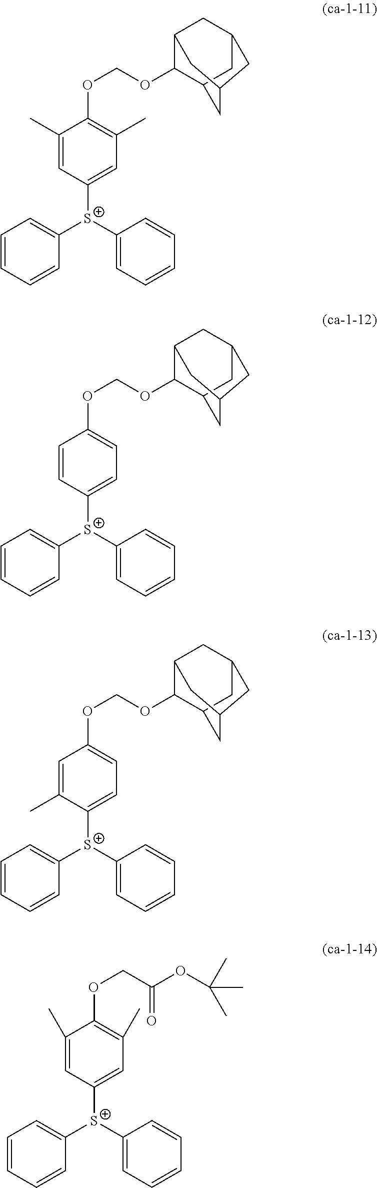 [SMILES: Cc1cc([S+](c2ccccc2)c2ccccc2)cc(C)c1OCOC1C2CC3CC(C2)CC1C3 ; c1ccc([S+](c2ccccc2)c2ccc(OCOC3C4CC5CC(C4)CC3C5)cc2)cc1 ; Cc1cc(OCOC2C3CC4CC(C3)CC2C4)ccc1[S+](c1ccccc1)c1ccccc1 ; Cc1cc([S+](c2ccccc2)c2ccccc2)cc(C)c1OCC(=O)OC(C)(C)C]

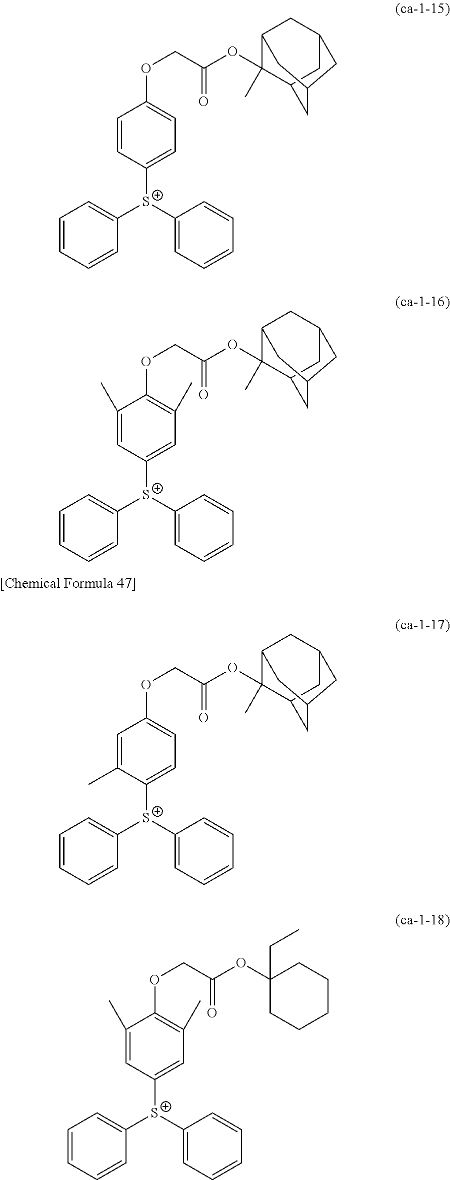 [SMILES: CC1(OC(=O)COc2ccc([S+](c3ccccc3)c3ccccc3)cc2)C2CC3CC(C2)CC1C3 ; Cc1cc([S+](c2ccccc2)c2ccccc2)cc(C)c1OCC(=O)OC1(C)C2CC3CC(C2)CC1C3 ; Cc1cc(OCC(=O)OC2(C)C3CC4CC(C3)CC2C4)ccc1[S+](c1ccccc1)c1ccccc1 ; CCC1(OC(=O)COc2c(C)cc([S+](c3ccccc3)c3ccccc3)cc2C)CCCCC1]

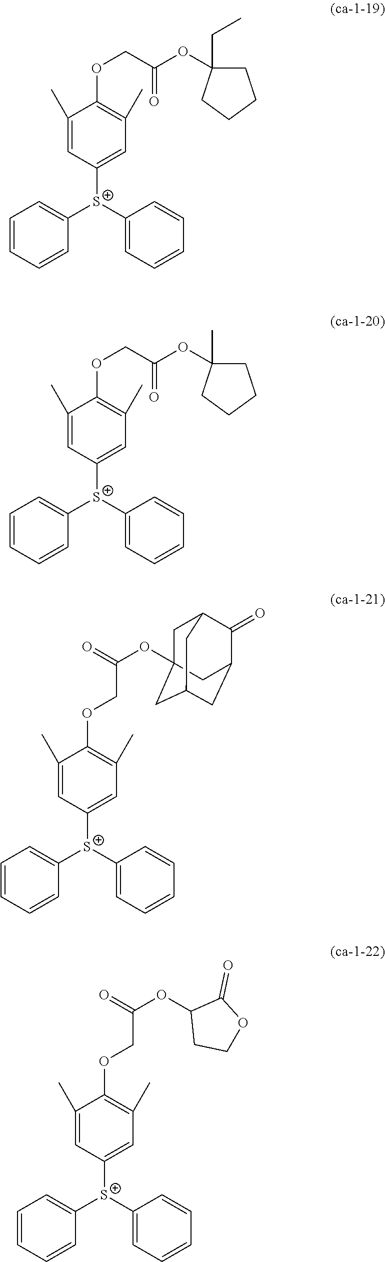 [SMILES: CCC1(OC(=O)COc2c(C)cc([S+](c3ccccc3)c3ccccc3)cc2C)CCCC1 ; Cc1cc([S+](c2ccccc2)c2ccccc2)cc(C)c1OCC(=O)OC1(C)CCCC1 ; Cc1cc([S+](c2ccccc2)c2ccccc2)cc(C)c1OCC(=O)OC12CC3CC(C1)C(=O)C(C3)C2 ; Cc1cc([S+](c2ccccc2)c2ccccc2)cc(C)c1OCC(=O)OC1CCOC1=O]

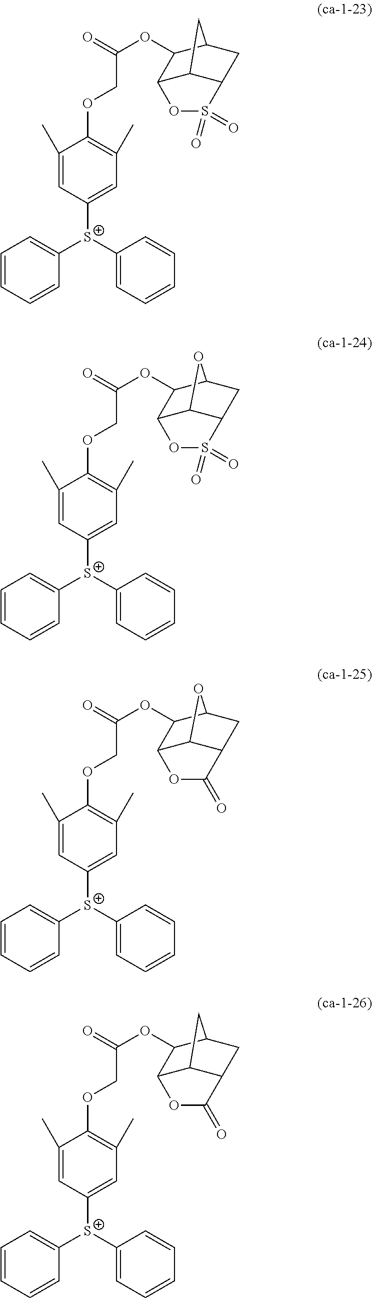 [SMILES: Cc1cc([S+](c2ccccc2)c2ccccc2)cc(C)c1OCC(=O)OC1C2CC3C1OS(=O)(=O)C3C2 ; Cc1cc([S+](c2ccccc2)c2ccccc2)cc(C)c1OCC(=O)OC1C2CC3C(O2)C1OS3(=O)=O ; Cc1cc([S+](c2ccccc2)c2ccccc2)cc(C)c1OCC(=O)OC1C2CC3C(=O)OC1C3O2 ; Cc1cc([S+](c2ccccc2)c2ccccc2)cc(C)c1OCC(=O)OC1C2CC3C(=O)OC1C3C2]

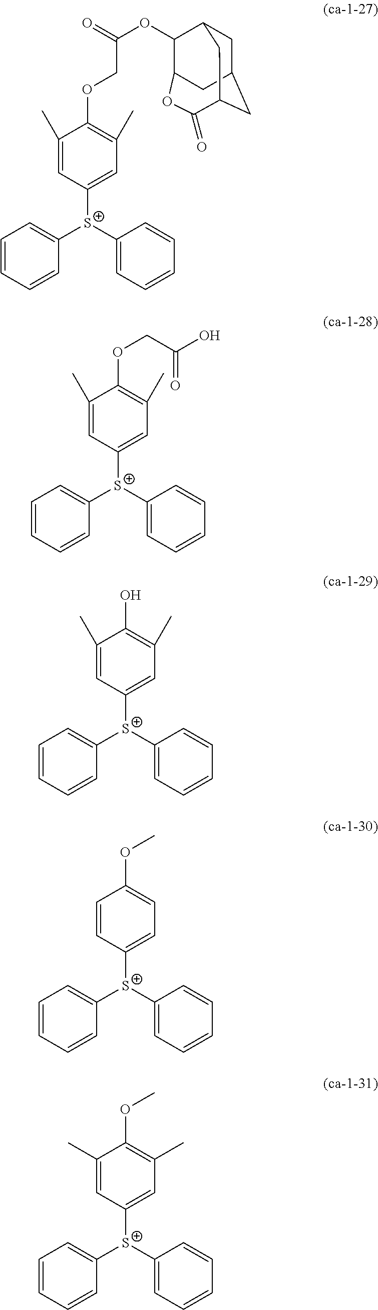 [SMILES: Cc1cc([S+](c2ccccc2)c2ccccc2)cc(C)c1OCC(=O)OC1C2CC3CC(C2)C(=O)OC1C3 ; Cc1cc([S+](c2ccccc2)c2ccccc2)cc(C)c1OCC(=O)O ; Cc1cc([S+](c2ccccc2)c2ccccc2)cc(C)c1O ; COc1ccc([S+](c2ccccc2)c2ccccc2)cc1 ; COc1c(C)cc([S+](c2ccccc2)c2ccccc2)cc1C]

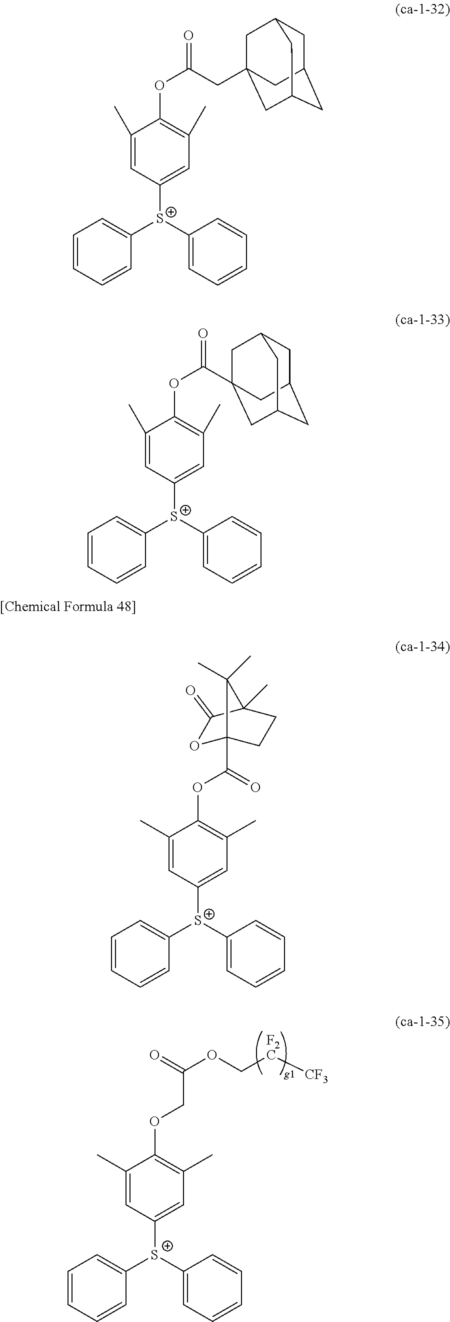

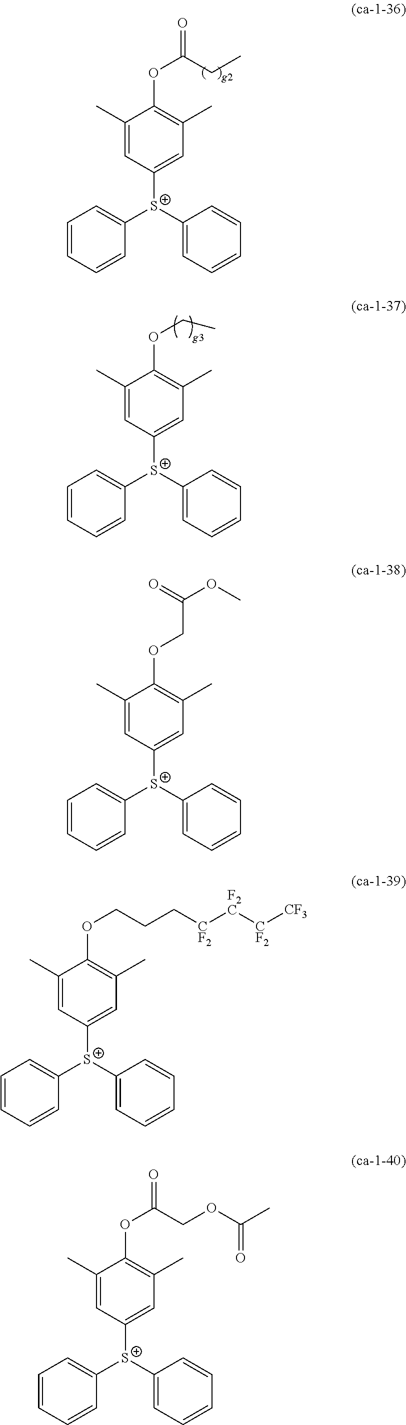

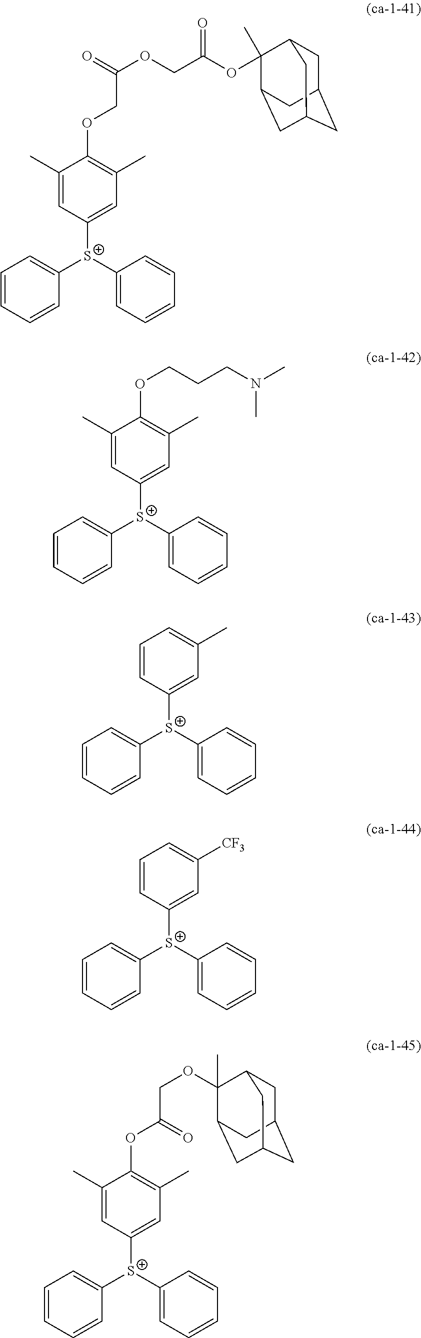 [SMILES: Cc1cc([S+](c2ccccc2)c2ccccc2)cc(C)c1OCC(=O)OCC(=O)OC1(C)C2CC3CC(C2)CC1C3 ; Cc1cc([S+](c2ccccc2)c2ccccc2)cc(C)c1OCCCN(C)C ; Cc1cccc([S+](c2ccccc2)c2ccccc2)c1 ; FC(F)(F)c1cccc([S+](c2ccccc2)c2ccccc2)c1 ; Cc1cc([S+](c2ccccc2)c2ccccc2)cc(C)c1OC(=O)COC1(C)C2CC3CC(C2)CC1C3]

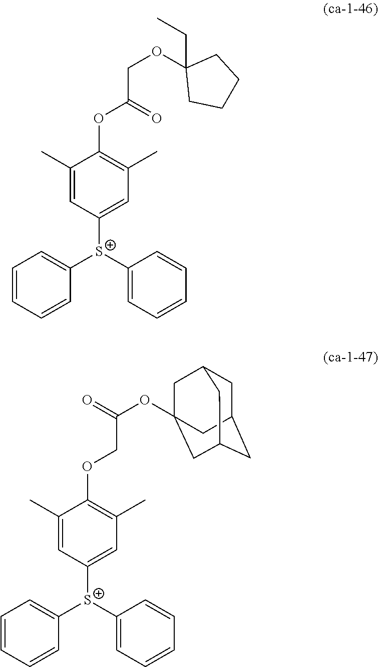 [SMILES: CCC1(OCC(=O)Oc2c(C)cc([S+](c3ccccc3)c3ccccc3)cc2C)CCCC1 ; Cc1cc([S+](c2ccccc2)c2ccccc2)cc(C)c1OCC(=O)OC12CC3CC(CC(C3)C1)C2]

[In the formulae, g1, g2, and g3 represent a repeating number, g1 represents an integer of 1 to 5, g2 represents an integer of 0 to 20, and g3 represents an integer of 0 to 20.]

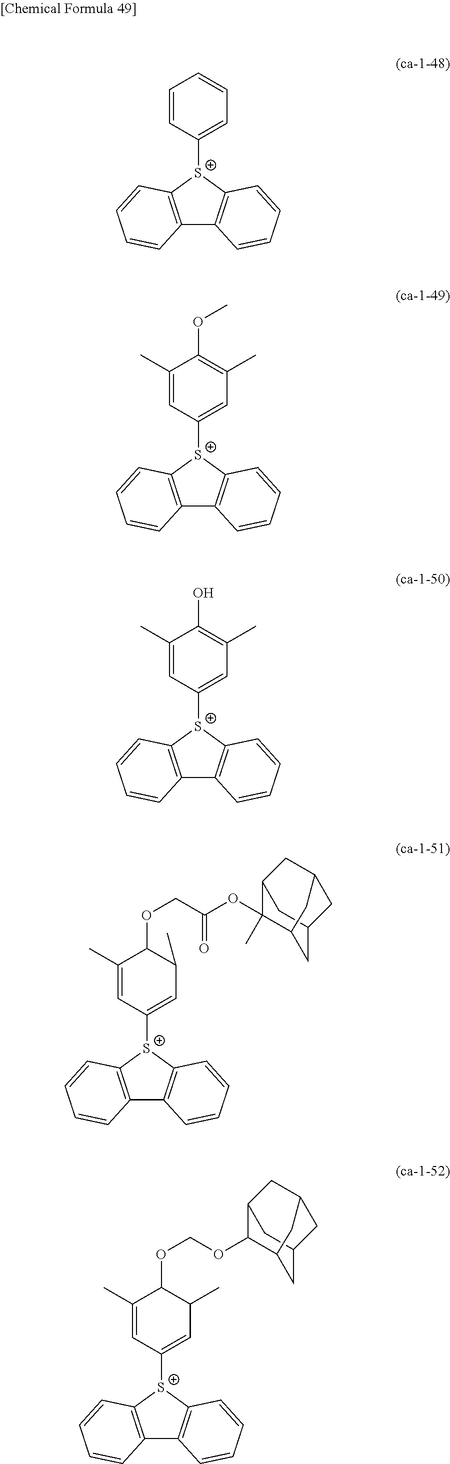 [SMILES: c1ccc(-[s+]2c3ccccc3c3ccccc32)cc1 ; COc1c(C)cc(-[s+]2c3ccccc3c3ccccc32)cc1C ; Cc1cc(-[s+]2c3ccccc3c3ccccc32)cc(C)c1O ; CC1=CC([s+]2c3ccccc3c3ccccc32)=CC(C)C1OCC(=O)OC1(C)C2CC3CC(C2)CC1C3 ; CC1=CC([s+]2c3ccccc3c3ccccc32)=CC(C)C1OCOC1C2CC3CC(C2)CC1C3]

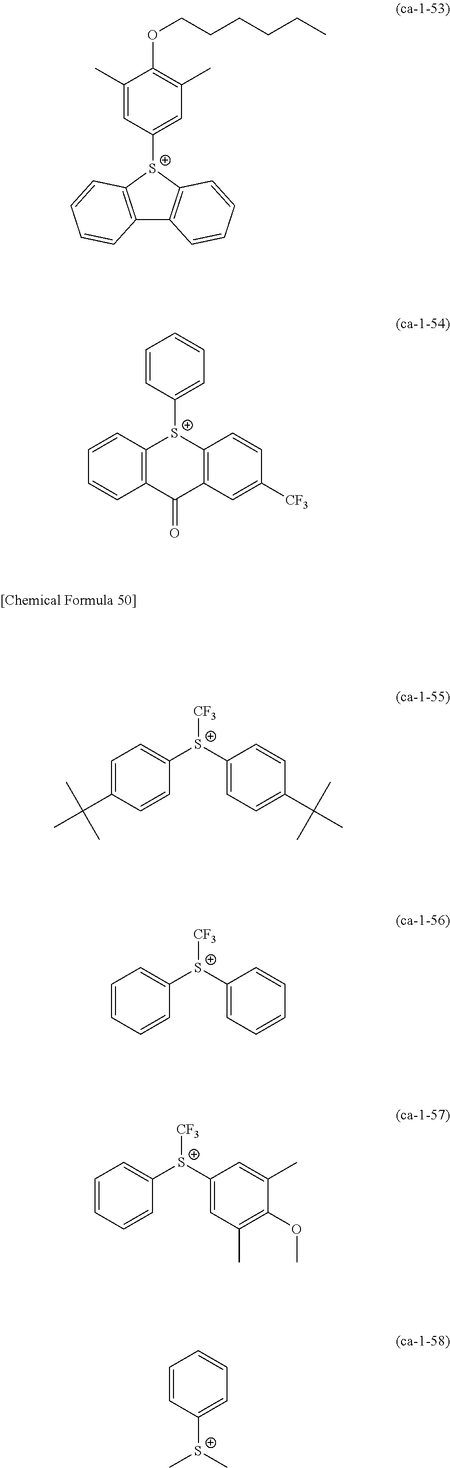

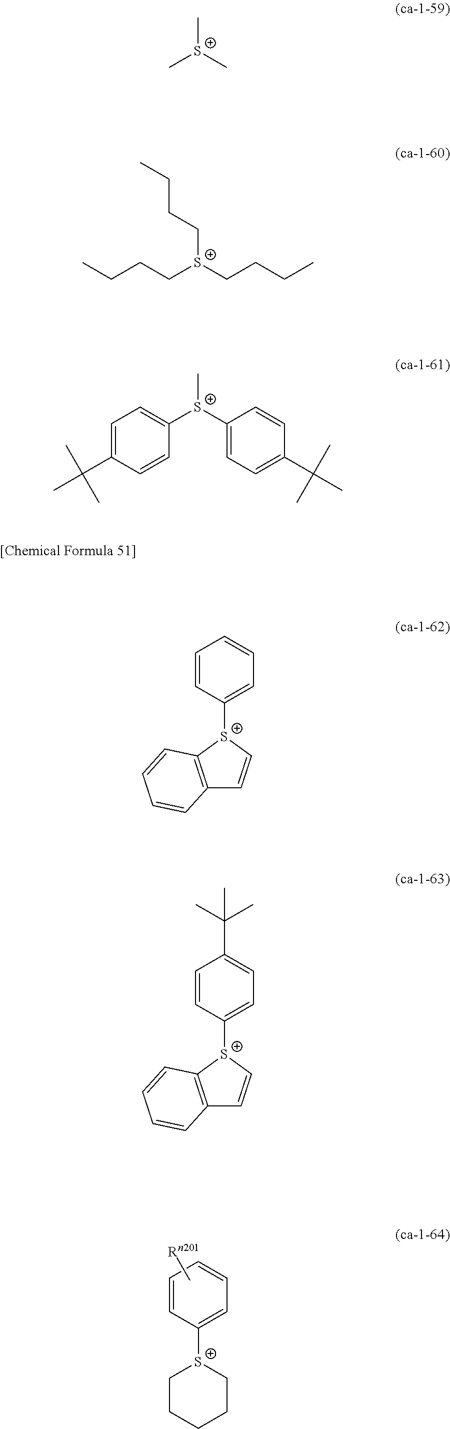

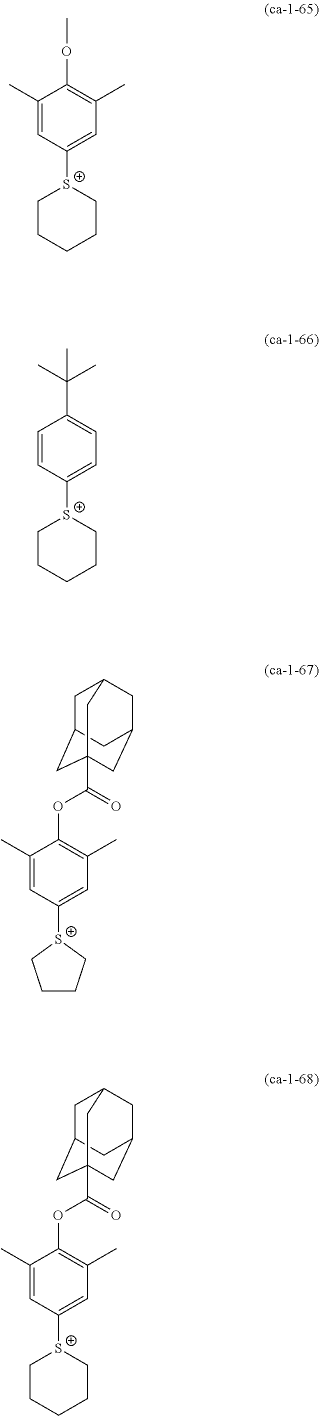

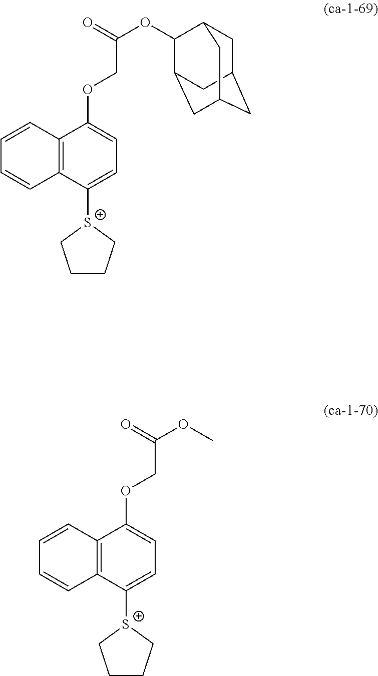 [SMILES: O=C(COc1ccc([S+]2CCCC2)c2ccccc12)OC1C2CC3CC(C2)CC1C3 ; COC(=O)COc1ccc([S+]2CCCC2)c2ccccc12]

[In the formulae, R″²⁰¹ represents a hydrogen atom or a substituent, and examples of the substituent include the same groups as those for the substituents which may be included in R²⁰¹ to R²⁰⁷ and R²¹⁰ to R²¹².]

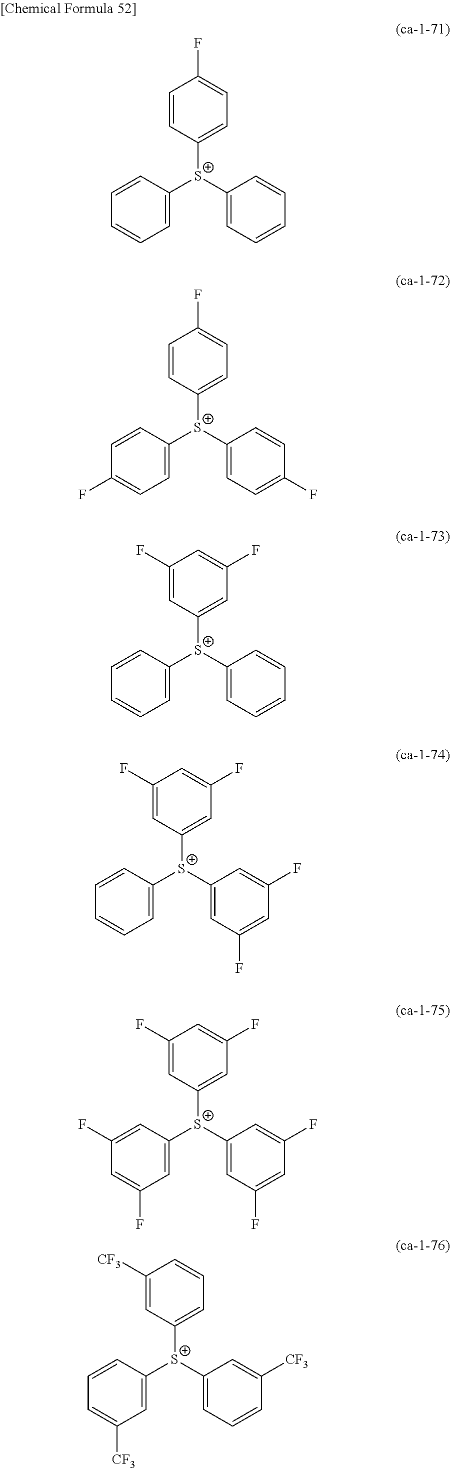 [SMILES: Fc1ccc([S+](c2ccccc2)c2ccccc2)cc1 ; Fc1ccc([S+](c2ccc(F)cc2)c2ccc(F)cc2)cc1 ; Fc1cc(F)cc([S+](c2ccccc2)c2ccccc2)c1 ; Fc1cc(F)cc([S+](c2ccccc2)c2cc(F)cc(F)c2)c1 ; Fc1cc(F)cc([S+](c2cc(F)cc(F)c2)c2cc(F)cc(F)c2)c1 ; FC(F)(F)c1cccc([S+](c2cccc(C(F)(F)F)c2)c2cccc(C(F)(F)F)c2)c1]

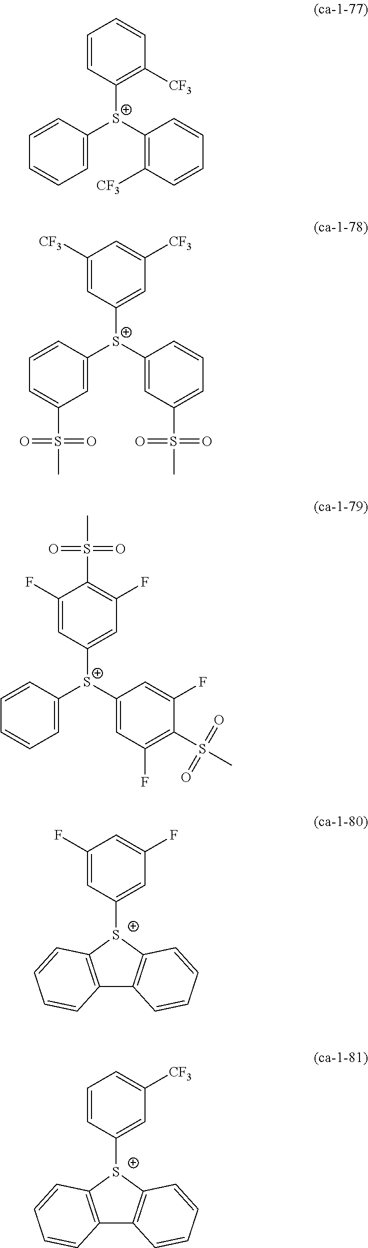 [SMILES: FC(F)(F)c1ccccc1[S+](c1ccccc1)c1ccccc1C(F)(F)F ; CS(=O)(=O)c1cccc([S+](c2cc(C(F)(F)F)cc(C(F)(F)F)c2)c2cccc(S(C)(=O)=O)c2)c1 ; CS(=O)(=O)c1c(F)cc([S+](c2ccccc2)c2cc(F)c(S(C)(=O)=O)c(F)c2)cc1F ; Fc1cc(F)cc(-[s+]2c3ccccc3c3ccccc32)c1 ; FC(F)(F)c1cccc(-[s+]2c3ccccc3c3ccccc32)c1]

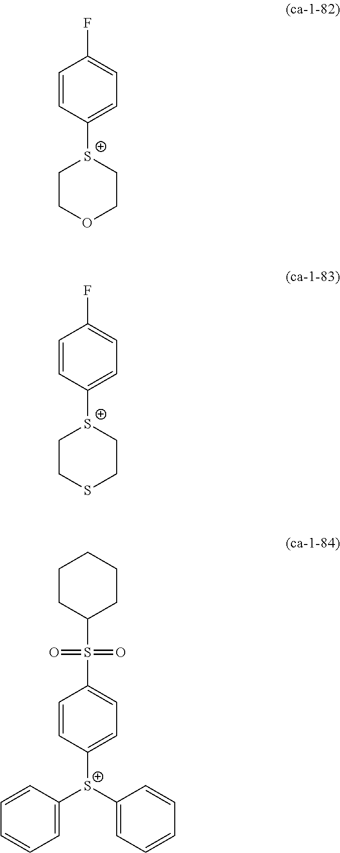 [SMILES: Fc1ccc([S+]2CCOCC2)cc1 ; Fc1ccc([S+]2CCSCC2)cc1 ; O=S(=O)(c1ccc([S+](c2ccccc2)c2ccccc2)cc1)C1CCCCC1]

Specific examples of suitable cations represented by Formula (ca-2) include a diphenyliodonium cation and a bis(4-tert-butylphenyl) iodonium cation.

Specific examples of suitable cations represented by Formula (ca-3) include cations each represented by Formulae (ca-3-1) to (ca-3-6).

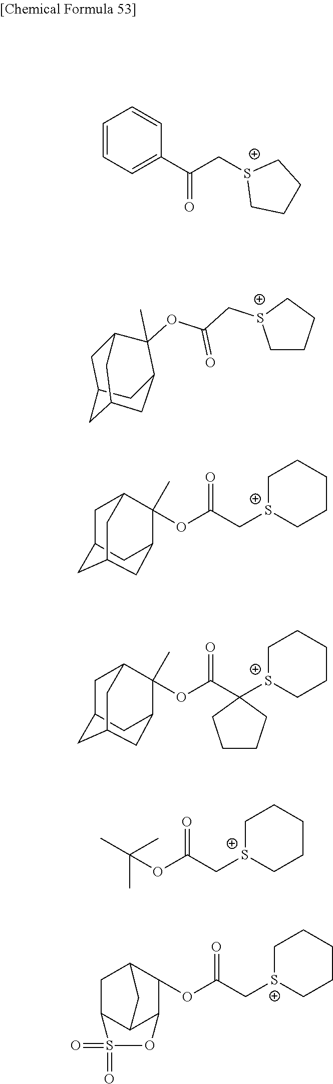 [SMILES: O=C(C[S+]1CCCC1)c1ccccc1 ; CC1(OC(=O)C[S+]2CCCC2)C2CC3CC(C2)CC1C3 ; CC1(OC(=O)C[S+]2CCCCC2)C2CC3CC(C2)CC1C3 ; CC1(OC(=O)C2([S+]3CCCCC3)CCCC2)C2CC3CC(C2)CC1C3 ; CC(C)(C)OC(=O)C[S+]1CCCCC1 ; O=C(C[S+]1CCCCC1)OC1C2CC3C1OS(=O)(=O)C3C2]

The organic cation as M^{m+} is preferably at least one selected from the group consisting of cations each represented by General Formulae (ca-1) to (ca-3). Among these, a cation represented by General Formula (ca-1) is more preferable.

In particular, from the viewpoint of increasing sensitivity, the suitable cation represented by Formula (ca-1) preferably has, as a substituent, an electron-withdrawing group such as a fluorine atom, a fluorinated alkyl group, or a sulfonyl group and is, for example, particularly preferably a cation selected from the group consisting of cations each represented by Chemical Formulae (ca-1-44) and (ca-1-71) to (ca-1-84).

### [Anion Moiety of Component (B0)]

In Formula (b0), R⁰ represents a fluorinated alkyl group having 1 to 5 carbon atoms or a fluorine atom, preferably a perfluoroalkyl group having 1 to 5 carbon atoms or a fluorine atom, and more preferably a fluorine atom.

In Formula (b0), Vb⁰ represents an alkylene group, a fluorinated alkylene group, or a single bond. The alkylene group and the fluorinated alkylene group as Vb⁰ have preferably 1 to 5 carbon atoms and more preferably 1 to 3 carbon atoms. Among these, it is particularly preferable that Vb⁰ represents an alkylene group having 1 to 3 carbon atoms or a fluorinated alkylene group having 1 to 3 carbon atoms. In a case of the fluorinated alkylene group having 1 to 3 carbon atoms, the number of fluorine atoms is preferably in a range of 1 to 3.

In Formula (b0), Yb⁰ represents a divalent linking group.

Examples of the divalent linking group as Yb⁰ include a divalent hydrocarbon group which may have a substituent.

In a case where Yb⁰ represents a divalent hydrocarbon group which may have a substituent, the hydrocarbon group may be an aliphatic hydrocarbon group or an aromatic hydrocarbon group.

Aliphatic hydrocarbon group as Yb⁰

The aliphatic hydrocarbon group indicates a hydrocarbon group that has no aromaticity. The aliphatic hydrocarbon group may be saturated or unsaturated. In general, it is preferable that the aliphatic hydrocarbon group is saturated.

Examples of the aliphatic hydrocarbon group include a linear or branched aliphatic hydrocarbon group and an aliphatic hydrocarbon group having a ring in the structure thereof.

### Linear or Branched Aliphatic Hydrocarbon Group

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

The linear or branched aliphatic hydrocarbon group may have or may not have a substituent. Examples of the substituent include a fluorine atom, a fluorinated alkyl group having 1 to 5 carbon atoms which has been substituted with a fluorine atom, and a carbonyl group.

### Aliphatic Hydrocarbon Group Containing Ring in Structure Thereof

Examples of the aliphatic hydrocarbon group having a ring in the structure thereof include a cyclic aliphatic hydrocarbon group which may have a substituent having a heteroatom in the ring structure thereof (a group in which two hydrogen atoms have been removed from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in the middle of a linear or branched aliphatic hydrocarbon group. Examples of the linear or branched aliphatic hydrocarbon group include the same groups as those described above.

The cyclic aliphatic hydrocarbon group has preferably 3 to 20 carbon atoms and more preferably 3 to 12 carbon atoms.

The cyclic aliphatic hydrocarbon group may be a polycyclic group or a monocyclic group. The monocyclic alicyclic hydrocarbon group is preferably a group obtained by removing two hydrogen atoms from a monocycloalkane. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples of the polycycloalkane include adamantane, norbornane, isobornane, tricyclodecane, and tetracyclododecane.

The cyclic aliphatic hydrocarbon group may or may not have a substituent. Examples of the substituent include an alkyl group, an alkoxy group, a halogen atom, a halogenated alkyl group, a hydroxyl group, and a carbonyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is still more preferable.

As the halogen atom as the substituent, a fluorine atom is preferable.

Examples of the halogenated alkyl group as the substituent include groups in which some or all hydrogen atoms in the above-described alkyl groups are substituted with the above-described halogen atoms.

In the cyclic aliphatic hydrocarbon group, some carbon atoms constituting the ring structure thereof may be substituted with a substituent having a heteroatom. As the substituent having a heteroatom, —O—, —C(═O)—O—, —S—, —S(═O)₂—, or —S(═O)₂—O— is preferable.

### Aromatic Hydrocarbon Group as Yb⁰

The aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to carbon atoms, still more preferably 6 to 15 carbon atoms, and particularly preferably 6 to 12 carbon atoms. Here, the number of carbon atoms in a substituent is not included in the number of carbon atoms.

Specifically, as the aromatic ring, an aromatic hydrocarbon ring such as benzene, naphthalene, anthracene, and phenanthrene; and an aromatic heterocyclic ring in which some carbon atoms constituting the aromatic hydrocarbon ring have been substituted with heteroatoms are exemplary examples. Examples of the heteroatom in the aromatic heterocyclic rings include an oxygen atom, a sulfur atom, and a nitrogen atom. Specific examples of the aromatic heterocyclic ring include a pyridine ring and a thiophene ring. In addition, the aromatic ring described here may be a condensed ring having one or more aromatic rings. Examples of the aromatic ring constituting the condensed ring include the aromatic hydrocarbon ring and the aromatic heterocyclic ring.

In the aromatic hydrocarbon group as Yb⁰, a hydrogen atom of the aromatic hydrocarbon group may be substituted with a substituent. For example, the hydrogen atom bonded to the aromatic ring in the aromatic hydrocarbon group may be substituted with a substituent. Examples of substituents include an alkyl group, an alkoxy group, a halogen atom, a halogenated alkyl group, and a hydroxyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group, the halogen atom, and the halogenated alkyl group as the substituents, the groups described as the substituents that substitute a hydrogen atom in the cyclic aliphatic hydrocarbon group are exemplary examples.

Specific examples of the aromatic hydrocarbon group as Yb⁰ include a group in which two hydrogen atoms have been removed from the aromatic hydrocarbon ring or the aromatic heterocyclic ring (an arylene group or a heteroarylene group); a group in which two hydrogen atoms have been removed from an aromatic compound (for example, biphenyl or fluorene) having two or more aromatic rings; a group in which one hydrogen atom of a group (an aryl group or a heteroaryl group) obtained by removing one hydrogen atom from the aromatic hydrocarbon ring or the aromatic heterocyclic ring is substituted with an alkylene group (for example, a group in which one hydrogen atom has been further removed from an aryl group in an arylalkyl group such as a benzyl group, a phenethyl group, a 1-naphthylmethyl group, a 2-naphthylmethyl group, a 1-naphthylethyl group, or a 2-naphthylethyl group); a condensed cyclic group in which two hydrogen atoms have been removed from a condensed ring having one or more aromatic rings, such as a polycyclic aromatic cyclic group having the aromatic rings being condensed with each other or an aromatic ring-aliphatic hydrocarbon ring-condensed cyclic group having the aromatic ring and the aliphatic hydrocarbon ring being condensed with each other; and a group in which one hydrogen atom of a group obtained by removing one hydrogen atom from a condensed ring having one or more aromatic rings is substituted with “divalent linking group having an oxygen atom”.

The number of carbon atoms in the alkylene group bonded to the aryl group or the heteroaryl group is preferably in a range of 1 to 4, more preferably 1 or 2, and particularly preferably 1.

Examples of “divalent linking group having an oxygen atom” include a non-hydrocarbon-based oxygen atom-containing linking group such as an oxygen atom (ether bond: —O—), an ester bond (—C(═O)—O—), an oxycarbonyl group (—O—C(═O))—), an amide bond (~C(═O)—NH—), a carbonyl group (—C(═O)—), or a carbonate bond (—O—C(═O)—O—); and a combination of the non-hydrocarbon-based oxygen atom-containing linking group and a divalent hydrocarbon group which may have a substituent. Further, a sulfonyl group (—SO₂—) may be further linked to the combination.

Examples of such “divalent linking group having an oxygen atom” include linking groups each represented by General Formulae (y-a1-1) to (y-a1-8). Further, in General Formulae (y-a1-1) to (y-a1-8), V*10) in General Formulae (y-a1-1) to (y-a1-8) is bonded to a group obtained by removing one hydrogen atom from a condensed ring having one or more of the aromatic rings.

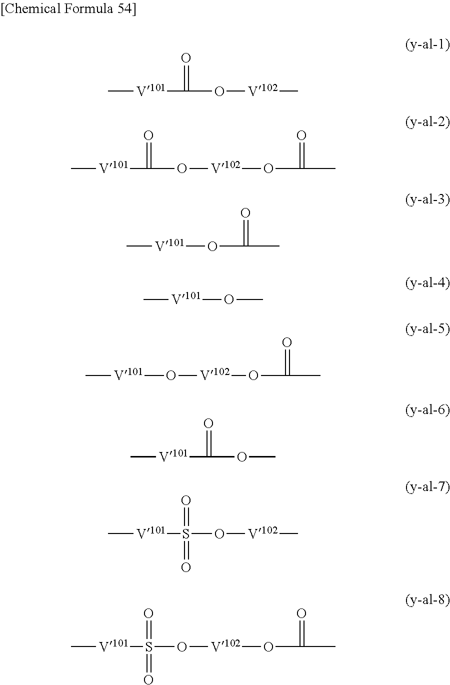

[In the formula, V′¹⁰¹ represents an alkylene group having 1 to 5 carbon atoms or a single bond. V′¹⁰² is a divalent saturated hydrocarbon group having 1 to 30 carbon atoms]

As the divalent saturated hydrocarbon group as V′¹⁰², an alkylene group having 1 to 30 carbon atoms is preferable, an alkylene group having 1 to 10 carbon atoms is more preferable, and an alkylene group having 1 to 5 carbon atoms is still more preferable.

The alkylene group as V′¹⁰¹ and V′¹⁰² may be a linear alkylene group or a branched alkylene group, and a linear alkylene group is preferable.

Specific examples of the alkylene group as V′¹⁰¹ and V*¹⁰² include a methylene group [—CH₂—]; an alkylmethylene group such as —CH(CH₃)—, —CH(CH₂CH₃)—, —C(CH₃)₂—, —C(CH₃)(CH₂CH₃)—, —C(CH₃)(CH₂CH₂CH₃)—, or —C(CH₂CH₃)₂—; an ethylene group [—CH₂CH₂—]; an alkylethylene group such as —CH(CH₃)CH₂, —CH(CH₃)CH(CH₃)—, —C(CH₃)₂CH₂—, or —CH(CH₂CH₃)CH₂; a trimethylene group (n-propylene group) [—CH₂CH₂CH₂—]; an alkyltrimethylene group such as —CH(CH₃)CH₂CH₂— or —CH₂CH(CH₃)CH₂—; a tetramethylene group [—CH₂CH₂CH₂CH₂—]; an alkyltetramethylene group such as —CH(CH₃)CH₂CH₂CH₂— or —CH₂CH(CH₃)CH₂CH₂; and a pentamethylene group [—CH₂CH₂CH₂CH₂CH₂—].

Further, a part of methylene groups in the alkylene group as V′¹⁰¹ and V′¹⁰² may be substituted with a divalent aliphatic cyclic group having 5 to 10 carbon atoms. The aliphatic cyclic group is preferably a cyclohexylene group, a 1,5-adamantylene group, or a 2,6-adamantylene group.

The divalent linking group as Yb⁰ is preferably a divalent hydrocarbon group which may have a substituent, more preferably an aliphatic hydrocarbon group (which may have a substituent) having a ring in the structure or an aromatic hydrocarbon group (which may have a substituent), still more preferably a polycyclic alicyclic hydrocarbon group (which may have a substituent) or an aromatic hydrocarbon group (which May have a substituent), and particularly preferably an aromatic hydrocarbon group which may have a substituent from the viewpoint of further achieving high sensitivity.

Among these, it is preferable that Yb⁰ represents (X) a condensed cyclic group obtained by removing two hydrogen atoms from a condensed ring having one or more aromatic rings or (Y) a group in which one hydrogen atom of a group obtained by removing one hydrogen atom from a condensed ring having one or more aromatic rings is substituted with “divalent linking group having an oxygen atom”.

In regard to (X) described above, suitable examples of the condensed cyclic group obtained by removing two hydrogen atoms from a condensed ring having one or more aromatic rings include a polycyclic aromatic cyclic group in which the aromatic rings are condensed and an aromatic ring-aliphatic hydrocarbon ring-condensed cyclic group in which the aromatic ring and the aliphatic hydrocarbon ring are condensed. Among these, an aromatic ring-aliphatic hydrocarbon ring-condensed cyclic group is more preferable.

Examples of the polycyclic aromatic cyclic group include a group obtained by removing two hydrogen atoms from naphthalene, anthracene, phenanthrene, biphenyl, or a polycyclic aromatic heterocyclic ring in which some carbon atoms constituting these aromatic rings are substituted with heteroatoms. Examples of the heteroatoms in the polycyclic aromatic heterocyclic rings include an oxygen atom, a sulfur atom, and a nitrogen atom.

Examples of the aromatic ring-aliphatic hydrocarbon ring-condensed cyclic group include fluorene; and a group in which one or more aromatic rings are condensed with a polycycloalkane having a crosslinked ring polycyclic skeleton.

Specific examples of the crosslinked ring polycycloalkane include a bicycloalkane such as bicyclo[2.2.1]heptane (norbornane) and bicyclo[2.2.2]octane.

Among these, the aromatic ring-aliphatic hydrocarbon ring-condensed cyclic group is preferably a group having a condensed ring, in which two or three aromatic rings are condensed with a bicycloalkane and more preferably a group having a condensed ring, in which two or three aromatic rings are condensed with bicyclo[2.2.2]octane.

Specific examples of the condensed ring having one or more aromatic rings as Yb⁰ include a condensed ring represented by Chemical Formula (r-dr-1) and a condensed ring represented by Chemical Formula (r-dr-2).

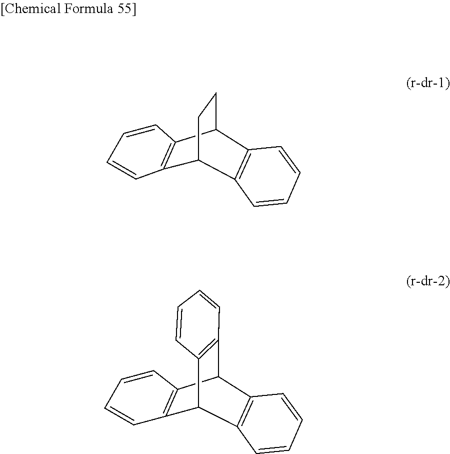

Examples of the condensed ring having one or more aromatic rings in (Y) described above include the same rings as those for the condensed ring having one or more aromatic rings in (X) described above, and suitable examples thereof include a polycyclic aromatic cyclic group in which aromatic rings are condensed and an aromatic ring-aliphatic hydrocarbon ring-condensed cyclic group in which the aromatic ring and the aliphatic hydrocarbon ring are condensed. Among these, an aromatic ring-aliphatic hydrocarbon ring-condensed cyclic group is more preferable.

In regard to (Y) described above, “divalent linking group having an oxygen atom” is preferably a divalent linking group having an ester bond or a divalent linking group having an ether bond, more preferably a linking group represented by any of Formulae (y-a1-1) to (y-a1-8), and still more preferably a linking group represented by Formula (y-a1-6).

In Formula (b0), Rpg represents an acid dissociable group represented by General Formula (a0-pg). That is, the anion moiety of the component (B0) includes an acid decomposable group whose polarity is increased by the action of an acid. Specifically, the acid dissociable group Rpg is bonded to the carbonyloxy group in Formula (b0) to form an acid decomposable group. Therefore, in a case where the carbon atom (C^{A}) forming the ring skeleton of the cyclic alkene A, which is bonded to the carbonyloxy group in Formula (b0), is a secondary carbon atom, the carbon-carbon unsaturated bond of the ring skeleton of the cyclic alkene A is formed between the carbon atom (C″^{α}) at the adjacent position of the carbon atom (C^{A}) (α-position of the carbon atom (C^{A})) and the carbon atom (C^{β}) at the adjacent position of the carbon atom (C^{α}) (β-position of the carbon atom (C^{A})). In a case where the carbon atom (C^{A}) is a tertiary carbon atom, the position of the carbon-carbon unsaturated bond of the ring skeleton of the cyclic alkene A is not particularly limited. From the viewpoint of improving acid dissociation properties, in a case where the carbon atom (C^{A}) is a tertiary carbon atom, it is preferable that the carbon-carbon unsaturated bond of the ring skeleton of the cyclic alkene A is formed between the carbon atom (C^{α}) and the carbon atom (C^{β}).

In Formula (a0-pg), A represents a cyclic alkene having only one carbon-carbon unsaturated bond in the ring skeleton. The cyclic alkene as A may be a polycyclic group or a monocyclic group.

As the cyclic alkene which is a monocyclic group, a group obtained by removing one hydrogen atom from a monocycloalkene is preferable. The monocycloalkene preferably has 3 to 6 carbon atoms, and specific examples thereof include cyclopentene and cyclohexene.

A group obtained by removing one hydrogen atom from a polycycloalkene is preferable as the cyclic alkene which is a polycyclic group, and a polycycloalkane having 7 to 12 carbon atoms is preferable as the polycycloalkane, and specific examples thereof include adamantene, norbornene, isobornene, tricyclodecene, and tetracyclododecene.

Among these, as the cyclic alkene represented by A, a monocyclic cyclic alkene is preferable, a group obtained by removing one hydrogen atom from a monocycloalkene is more preferable, and a group obtained by removing one hydrogen atom from cyclopentene or cyclohexene is still more preferable.

In Formula (a0-pg), examples of the monovalent hydrocarbon group having 1 to 20 carbon atoms as Ra⁰¹ include a chain-like alkyl group and a chain-like alkenyl group.

The chain-like alkyl group as Ra⁰¹ may be linear or branched.

The linear alkyl group has preferably 1 to 20 carbon atoms, more preferably 1 to 15 carbon atoms, and most preferably 1 to 10 carbon atoms. Specific examples thereof include a methyl group, an ethyl group, a propyl group, a butyl group, a pentyl group, a hexyl group, a heptyl group, an octyl group, a nonyl group, and a decyl group.

The branched alkyl group has preferably 3 to 20 carbon atoms, more preferably 3 to 15 carbon atoms, and most preferably 3 to 10 carbon atoms. Specific examples thereof include an isopropyl group, an isobutyl group, a tert-butyl group, an isopentyl group, a neopentyl group, a 1,1-diethylpropyl group, and a 2,2-dimethylbutyl group.

The chain-like alkenyl group as Ra⁰¹ may be linear or branched, and has preferably 2 to 20 carbon atoms, more preferably 2 to 10 carbon atoms, and still more preferably 2 to 5 carbon atoms. Examples of the linear alkenyl group include a vinyl group, a propenyl group (allyl group), and a butenyl group. Examples of the branched alkenyl group include a 1-methylvinyl group, a 2-methylvinyl group, a 1-methylpropenyl group, and a 2-methylpropenyl group.

Among these, Ra⁰¹ represents preferably a chain-like alkyl group, more preferably a chain-like alkyl group having 1 to 20 carbon atoms, still more preferably a chain-like alkyl group having 1 to 10 carbon atoms, and even still more preferably a chain-like alkyl group having 1 to 5 carbon atoms.

In Formula (a0-pg), m1 represents preferably 0 to 10, more preferably 0 to 5, still more preferably 0 to 2, and may be 0.

In Formula (a0-pg), m2 represents preferably 1 to 10, more preferably 1 to 5, still more preferably 1 or 2, and may be 1.

Here, in Formula (a0-pg), m1+m2≤(total number of carbon atoms constituting ring skeleton of cyclic alkene as A-1 (total number-1)).

In Formula (a0-pg), from the viewpoint of achieving high sensitivity, it is preferable that an iodine atom is bonded to at least one of the carbon atoms constituting the carbon-carbon unsaturated bond in a ring skeleton of a cyclic alkene as A, more preferable that the carbon-carbon unsaturated bond of the ring skeleton of the cyclic alkene A is formed between the carbon atom (C^{α}) at an adjacent position (α-position of the carbon atom (C^{A})) of the carbon atom (C^{A}) forming the ring skeleton of the cyclic alkene A, which is bonded to the carbonyloxy group in Formula (b0), and the carbon atom (C^{β}) at an adjacent position (β-position of the carbon atom (C^{A})) of the carbon atom (C^{α}) and an iodine atom is bonded to at least one of the carbon atom (C^{α}) and the carbon atom (C^{β}), and still more preferable that the carbon-carbon unsaturated bond of the ring skeleton of the cyclic alkene A is formed between the carbon atom (C^{α}) and the carbon atom (C^{β}) and an iodine atom is bonded to the carbon atom (C^{β}) from the viewpoint of achieving high sensitivity and improving the stability of the component (B0) in the resist film. In a case where an iodine atom is bonded to the carbon atom (C^{β}), the stability of a carbocation formed by the dissociation of the cyclic alkene A by the action of an acid is enhanced, the dissociation rate of Rpg from the anion moiety of the component (B0) is increased, and thus high sensitivity is likely to be achieved.

In Formula (a0-pg), in a case where m1 represents an integer of 2 or greater, a plurality of Ra⁰¹'s may be the same as or different from each other.

In the present embodiment, the component (B0) may be a compound represented by General Formula (b0-t1).

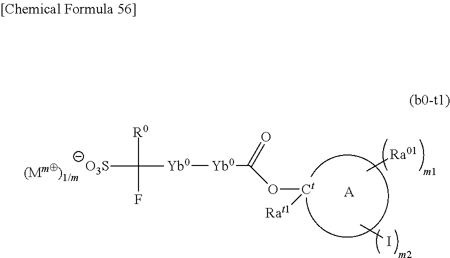

[In Formula (b0-t1), M^{m+} represents an m-valent organic cation. m represents an integer of 1 or greater. R⁰ represents a fluorinated alkyl group having 1 to 5 carbon atoms or a fluorine atom. Vb⁰ represents an alkylene group, a fluorinated alkylene group, or a single bond. Yb⁰ represents a divalent linking group. A represents a cyclic alkene having only one carbon-carbon unsaturated bond in a ring skeleton. C′ represents a tertiary carbon atom. Ra^{t1} represents a linear or branched alkyl group having 1 to 12 carbon atoms. Ra⁰¹ represents a monovalent hydrocarbon group having 1 to 20 carbon atoms. 1 represents an iodine atom, m1 represents an integer of 0 to 20. m2 represents an integer of 1 to 20. Here, m1+m2≤(a total number of carbon atoms constituting the ring skeleton of the cyclic alkene as A-1) is satisfied. In a case where m1 represents an integer of 2 or greater, a plurality of Ra⁰¹'s may be the same as or different from each other.]

In Formula (b0-t1), examples of the linear alkyl group having 1 to 12 carbon atoms as Ra^{t1} include a methyl group, an ethyl group, a propyl group, a butyl group, a pentyl group, a hexyl group, a heptyl group, an octyl group, a nonyl group, and a decyl group. Among these, a linear alkyl group having 1 to 5 carbon atoms is preferable, a methyl group, an ethyl group, or a butyl group is more preferable, and a methyl group or an ethyl group is still more preferable.

In Formula (b0-(1), examples of the branched alkyl group having 1 to 12 carbon atoms as Ra^{t1} include an isopropyl group, an isobutyl group, a tert-butyl group, an isopentyl group, a neopentyl group, a 1,1-diethylpropyl group, and a 2,2-dimethylbutyl group. Among these, a branched alkyl group having 3 to 5 carbon atoms is preferable, and an isopropyl group is more preferable.

In the present embodiment, it is preferable that the component (B0) is a compound represented by General Formula (b0-t1-1).

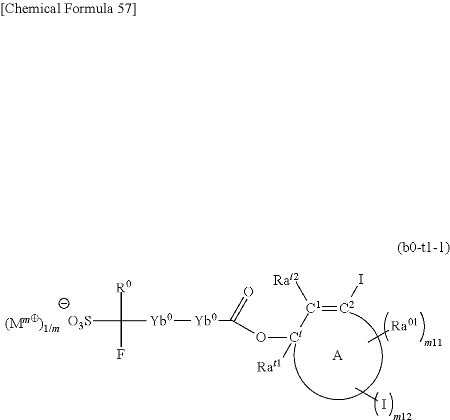

[In Formula (b0-(1-1), M^{m+} represents an m-valent organic cation, m represents an integer of 1 or greater. R⁰ represents a fluorinated alkyl group having 1 to carbon atoms or a fluorine atom. Vb⁰ represents an alkylene group, a fluorinated alkylene group, or a single bond. Yb⁰ represents a divalent linking group, A represents a cyclic alkene having only one carbon-carbon unsaturated bond in a ring skeleton. C^{t} represents a tertiary carbon atom. C¹ and C² each independently represent a carbon atom constituting the carbon-carbon unsaturated bond of the cyclic alkene as A. Ra^{t1} represents a linear or branched alkyl group having 1 to 12 carbon atoms. Ra^{t2} represents a hydrogen atom or a monovalent hydrocarbon group having 1 to 20 carbon atoms. Ra⁰¹ represents a monovalent hydrocarbon group having 1 to 20 carbon atoms. I represents an iodine atom. m11 represents an integer of 0 to 20. m12 represents an integer of 0 to 19. Here, m11+m12≤(the total number of carbon atoms constituting the ring skeleton of the cyclic alkene as A-3) is satisfied. In a case where m11 represents an integer of 2 or greater, a plurality of Ra⁰¹'s may be the same as or different from each other.]

In Formula (b0-t1-1), the monovalent hydrocarbon group having 1 to 20 carbon atoms as Ra^{t2} is the same as the monovalent hydrocarbon group having 1 to 20 carbon atoms as Ra⁰¹ in Formula (a0-pg). Among these, it is preferable that Ra^{t2} represents a hydrogen atom.

In Formula (b0-(1-1), m11 represents preferably 0 to 10, more preferably 0 to 5, still more preferably 0 to 2, and may be 0.

In Formula (b0-(1-1), m12 represents preferably 0 to 9, more preferably 0 to 4, still more preferably 0 or 1, and may be 0.

Here, in Formula (b0-t1-1), m11+m12≤(the total number of carbon atoms constituting the ring skeleton of the cyclic alkene as A-3 (total number−3).

In the present embodiment, the component (B0) may be a compound represented by General Formula (b0-s1).

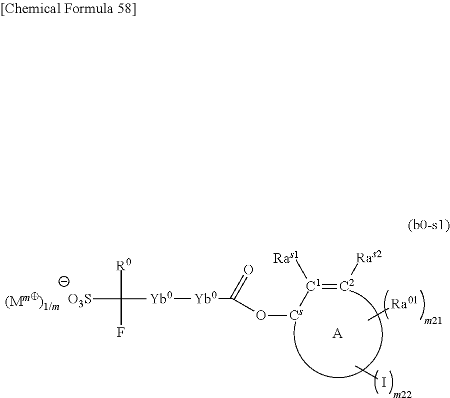

[In Formula (b0-s1), M^{m+} represents an m-valent organic cation. m represents an integer of 1 or greater. R⁰ represents a fluorinated alkyl group having 1 to 5 carbon atoms or a fluorine atom. Vb⁰ represents an alkylene group, a fluorinated alkylene group, or a single bond. Yb⁰ represents a divalent linking group. A represents a cyclic alkene having only one carbon-carbon unsaturated bond in a ring skeleton. C^{s} represents a secondary carbon atom. C¹ and C² each independently represent a carbon atom constituting the carbon-carbon unsaturated bond of the cyclic alkene as A. Ra^{s1} and Ra^{s2} each independently represent a hydrogen atom, an iodine atom, or a monovalent hydrocarbon group having 1 to 20 carbon atoms. Ra⁰¹ represents a monovalent hydrocarbon group having 1 to 20 carbon atoms. I represents an iodine atom. m21 represents an integer of 0 to 20. m22 represents an integer of 0 to 20. Here, m21+m22≤(the total number of carbon atoms constituting the ring skeleton of the cyclic alkene as A-3) is satisfied. In addition, in a case m22 represents 0, at least one of Ra^{s1} or Ra^{s2} represents an iodine atom. In a case where m21 represents an integer of 2 or greater, a plurality of Ra⁰¹'s may be the same as or different from each other.]

In Formula (b0-s1), the monovalent hydrocarbon group having 1 to 20 carbon atoms as Ra^{s1} and Ra^{s2} is the same as the monovalent hydrocarbon group having 1 to 20 carbon atoms as Ra⁰¹ in Formula (a0-pg). Among these, it is preferable that one of Ra^{s1} and Ra^{s2} represents an iodine atom and the other represents a hydrogen atom or a monovalent hydrocarbon group having 1 to 20 carbon atoms, more preferable that Ra^{s1} represents a hydrogen atom or a monovalent hydrocarbon group having 1 to 20 carbon atoms and Ra^{s2} represents an iodine atom, and still more preferable that Ra^{s1} represents a monovalent hydrocarbon group having 1 to 20 carbon atoms and Ra^{s2} represents an iodine atom.

In Formula (b0-s1), m21 represents preferably 0 to 10, more preferably 0 to 5, still more preferably 0 to 2, and may be 0.

In Formula (b0-s1), m22 represents preferably 0 to 10, more preferably 0 to 5, still more preferably 0 or 1, and may be 0.

Here, in Formula (b0-s1), m21+m22≤(the total number of carbon atoms constituting the ring skeleton of the cyclic alkene as A-3 (total number−3)).

In the present embodiment, the component (B0) may be a compound represented by General Formula (b0-s1-1).

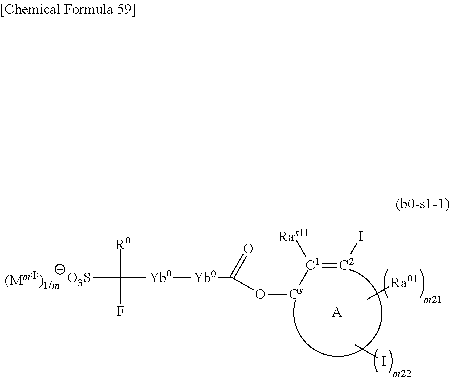

[In Formula (b0-s1-1), M^{m+} represents an m-valent organic cation. m represents an integer of 1 or greater. R⁰ represents a fluorinated alkyl group having 1 to carbon atoms or a fluorine atom. Vb⁰ represents an alkylene group, a fluorinated alkylene group, or a single bond. Yb⁰ represents a divalent linking group. A represents a cyclic alkene having only one carbon-carbon unsaturated bond in a ring skeleton. C^{s} represents a secondary carbon atom. C¹ and C² each independently represent a carbon atom constituting the carbon-carbon unsaturated bond of the cyclic alkene as A. Ra^{s11} represents a hydrogen atom or a monovalent hydrocarbon group having 1 to 20 carbon atoms. Ra⁰¹ represents a monovalent hydrocarbon group having 1 to 20 carbon atoms. I represents an iodine atom. m21 represents an integer of 0 to 20. m22 represents an integer of 0 to 19. Here, m21+m22≤(the total number of carbon atoms constituting the ring skeleton of the cyclic alkene as A-3) is satisfied. In a case where m21 represents an integer of 2 or greater, a plurality of Ra⁰¹'s may be the same as or different from each other.]

In Formula (b0-s1-1), m21 represents preferably 0 to 10, more preferably 0 to 5, still more preferably 0 to 2, and may be 0.

In Formula (b0-s1-1), m22 represents preferably 0 to 9, more preferably 0 to 4, still more preferably 0 or 1, and may be 0.

In Formula (b0-s1-1), the monovalent hydrocarbon group having 1 to 20 carbon atoms as Ra^{s11} is the same as the monovalent hydrocarbon group having 1 to 20 carbon atoms as Ra⁰¹ in Formula (a0-pg). Among these, Ra^{s11} represents preferably a chain-like alkyl group having 1 to 10 carbon atoms, more preferably a linear alkyl group having 1 to 5 carbon atoms or a branched alkyl group having 3 to 5 carbon atoms, and still more preferably a linear alkyl group having 1 to 3 carbon atoms.

In the present embodiment, the component (B0) may be a compound represented by General Formula (b0-s1-2).

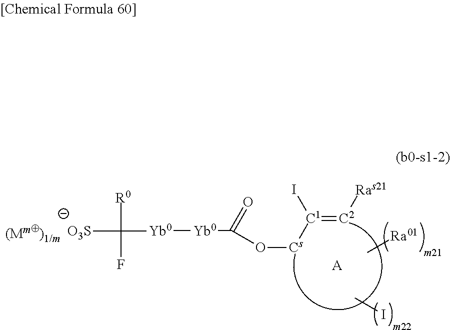

[In Formula (b0-s1-2), M^{m+} represents an m-valent organic cation. m represents an integer of 1 or greater. R⁰ represents a fluorinated alkyl group having 1 to carbon atoms or a fluorine atom. Vb⁰ represents an alkylene group, a fluorinated alkylene group, or a single bond. Yb⁰ represents a divalent linking group. A represents a cyclic alkene having only one carbon-carbon unsaturated bond in a ring skeleton. C^{s} represents a secondary carbon atom. C¹ and C² each independently represent a carbon atom constituting the carbon-carbon unsaturated bond of the cyclic alkene as A. Ra^{s21} represents a hydrogen atom or a monovalent hydrocarbon group having 1 to 20 carbon atoms. Ra⁰¹ represents a monovalent hydrocarbon group having 1 to 20 carbon atoms. 1 represents an iodine atom. m21 represents an integer of 0 to 20. m22 represents an integer of 0 to 19, Here, m21+m22≤(the total number of carbon atoms constituting the ring skeleton of the cyclic alkene as A-3) is satisfied. In a case where m21 represents an integer of 2 or greater, a plurality of Ra⁰¹'s may be the same as or different from each other.]

In Formula (b0-s1-2), the monovalent hydrocarbon group having 1 to 20 carbon atoms as Ra^{s21} is the same as the monovalent hydrocarbon group having 1 to 20 carbon atoms as Ra⁰¹ in Formula (a0-pg). Among these, it is preferable that Ra^{s21} represents a hydrogen atom.

In the present embodiment, the component (B0) may be a compound represented by General Formula (b0-s1-3).

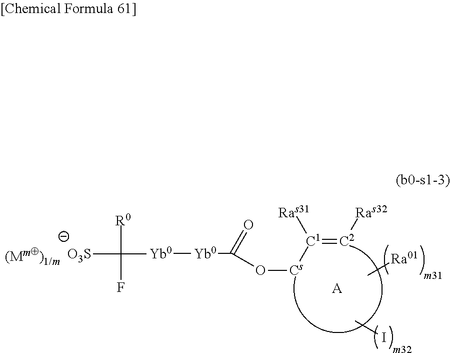

[In Formula (b0-s1-3), M^{m+} represents an m-valent organic cation. m represents an integer of 1 or greater. R⁰ represents a fluorinated alkyl group having 1 to carbon atoms or a fluorine atom. Vb⁰ represents an alkylene group, a fluorinated alkylene group, or a single bond. Yb⁰ represents a divalent linking group, A represents a cyclic alkene having only one carbon-carbon unsaturated bond in a ring skeleton. C^{s} represents a secondary carbon atom. C¹ and C² each independently represent a carbon atom constituting the carbon-carbon unsaturated bond of the cyclic alkene as A. Ra^{s31} and Ra^{s32} each independently represent a hydrogen atom or a monovalent hydrocarbon group having 1 to 20 carbon atoms. Ra⁰¹ represents a monovalent hydrocarbon group having 1 to 20 carbon atoms, m31 represents an integer of 0 to 20. m32 represents an integer of 1 to 20. Here, m31+m32≤(the total number of carbon atoms constituting the ring skeleton of the cyclic alkene as A-3) is satisfied. In a case where m31 represents an integer of 2 or greater, a plurality of Ra⁰¹'s may be the same as or different from each other.]

In Formula (b0-s1-3), m31 represents preferably 0) to 10, more preferably 0 to 5, still more preferably 0 to 2, and may be 0.

In Formula (b0-s1-3), m32 represents preferably 1 to 10, more preferably 1 to 5, still more preferably 1 or 2, and may be 1.

In Formula (b0-s1-3), the monovalent hydrocarbon group having 1 to 20 carbon atoms as Ra^{s31} and Ra^{s32} is the same as the monovalent hydrocarbon group having 1 to 20 carbon atoms as Ra⁰¹ in Formula (a0-pg). Among these, it is preferable that Ra^{s31} and Ra^{s32} each represent a hydrogen atom and more preferable that both represent a hydrogen atom.

Hereinafter, specific examples of the component (B0) will be described, but the present invention is not limited thereto.

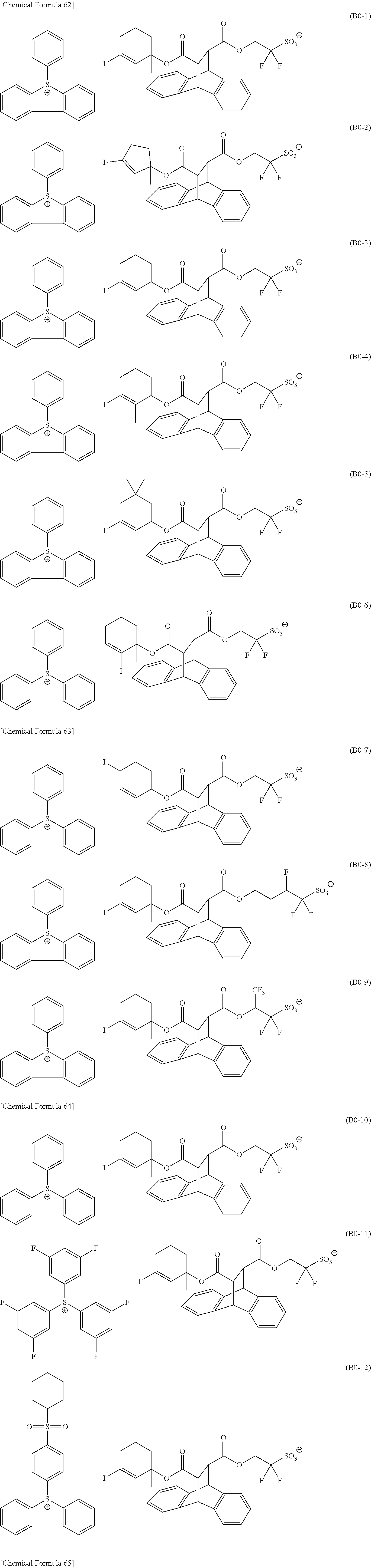

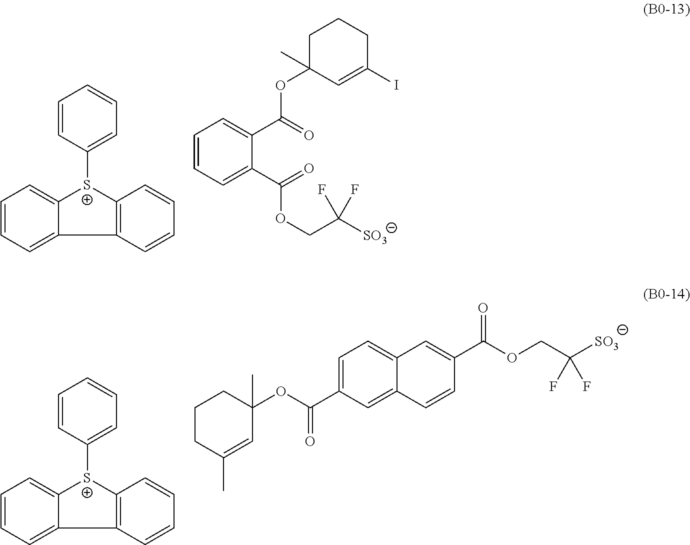

In the resist composition according to the present embodiment, the component (B0) may be used alone or may be used in a combination of two or more kinds thereof.

The content of the component (B0) in the resist composition according to the present embodiment is preferably in a range of 5 to 50 parts by mass, more preferably in a range of 10 to 40 parts by mass, and still more preferably in a range of 20 to 35 parts by mass with respect to 100 parts by mass of the component (A).

In a case where the content of the component (B0) is greater than or equal to the lower limits of the above-described preferable ranges, the sensitivity is improved in the formation of a resist pattern, and a decrease in film thickness during development is easily suppressed in the formation of a resist pattern. Meanwhile, in a case where the content thereof is less than or equal to the upper limits of the above-described preferable ranges, the sensitivity can be maintained more satisfactorily. Further, it is preferable that each component of the resist composition is dissolved in an organic solvent from the viewpoint that a uniform solution is easily obtained and the storage stability of the resist composition is improved.

<<Component (B1): Acid Generator Component Other than Component (B0)>>

The component (B) in the resist composition according to the present embodiment may contain an acid generator component other than the above-described component (B0) (hereinafter, also referred to as “component (B1)”).

The component (B1) is not particularly limited, and those which have been suggested as an acid generator for a chemically amplified resist composition in the related art can be used.

Examples of the acid generator include various acid generators, for example, onium salt-based acid generators such as iodonium salts and sulfonium salts; oxime sulfonate-based acid generators; diazomethane-based acid generators such as bisalkyl or bisaryl sulfonyl diazomethanes and poly(bis-sulfonyl)diazomethanes; nitrobenzyl sulfonate-based acid generators, imino sulfonate-based acid generators, and disulfone-based acid generators.

Examples of the onium salt-based acid generators include a compound represented by General Formula (b-1) (hereinafter, also referred to as “component (b-1)”), a compound represented by General Formula (b-2) (hereinafter, also referred to as “component (b-2)”), and a compound represented by General Formula (b-3) (hereinafter, also referred to as “component (b-3)”).

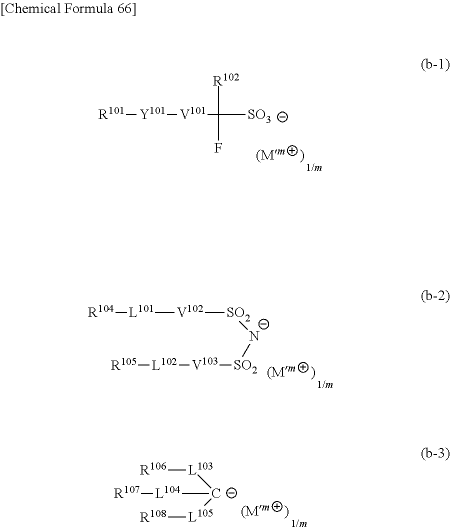

[In the formulae, R¹⁰¹ and R¹⁰⁴ to R¹⁰⁸ each independently represent a cyclic group which may have a substituent, a chain-like alkyl group which may have a substituent, or a chain-like alkenyl group which may have a substituent. R¹⁰⁴ and R¹⁰⁵ may be bonded to each other to form a ring structure. R¹⁰² represents a fluorinated alkyl group having 1 to 5 carbon atoms or a fluorine atom. Y¹⁰¹ represents a divalent linking group containing an oxygen atom or a single bond. V¹⁰¹ to V¹⁰³ each independently represent a single bond, an alkylene group, or a fluorinated alkylene group. Here, both Y¹⁰¹ and V¹⁰¹ do not form a single bond. L¹⁰¹ and L¹⁰² each independently represent a single bond or an oxygen atom. L¹⁰³ to L¹⁰⁵ each independently represent a single bond, —CO—, or —SO₂—. m represents an integer of 1 or greater, and M′^{m+} represents an m-valent onium cation.]

### {Anion Moiety}

Anions in Component (b-1)

In Formula (b-1), R¹⁰¹ represents a cyclic group which may have a substituent, a chain-like alkyl group which may have a substituent, or a chain-like alkenyl group which may have a substituent.

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. In addition, it is preferable that the aliphatic hydrocarbon group is saturated.

The aromatic hydrocarbon group as R¹⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring of the aromatic hydrocarbon group as R¹⁰¹ include benzene, fluorene, naphthalene, anthracene, phenanthrene, biphenyl, or an aromatic heterocyclic ring in which some carbon atoms constituting any of these aromatic rings have been substituted with heteroatoms. Examples of the heteroatom in the aromatic heterocyclic rings include an oxygen atom, a sulfur atom, and a nitrogen atom.

Specific examples of the aromatic hydrocarbon group as R¹⁰¹ include a group obtained by removing one hydrogen atom from the aromatic ring (an aryl group such as a phenyl group or a naphthyl group) and a group in which one hydrogen atom of the aromatic ring is substituted with an alkylene group (for example, a benzyl group, a phenethyl group, or a 1-naphthylmethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R¹⁰¹ include an aliphatic hydrocarbon group having a ring in the structure thereof.

Examples of the aliphatic hydrocarbon group having a ring in the structure thereof include an alicyclic hydrocarbon group (group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclodecane, or tetracyclododecane; and a polycycloalkane having a condensed ring polycyclic skeleton such as a cyclic group having a steroid skeleton are preferable as the polycycloalkane.

Among these examples, as the cyclic aliphatic hydrocarbon group as R¹⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is still more preferable, and an adamantyl group is particularly preferable.

The linear aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and most preferably 1 to 3 carbon atoms. As the linear aliphatic hydrocarbon group, a linear alkylene group is preferable. Specific examples thereof include a methylene group [—CH₂—], an ethylene group [—(CH₂)₂—), a trimethylene group [—(CH₂)₃—], a tetramethylene group [—(CH₂)₄—], and a pentamethylene group [—(CH₂)₅—].

The branched aliphatic hydrocarbon group which may be bonded to the alicyclic hydrocarbon group has preferably 2 to 10 carbon atoms, more preferably 3 to 6 carbon atoms, still more preferably 3 or 4 carbon atoms, and most preferably 3 carbon atoms. As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

Further, the cyclic hydrocarbon group as R¹⁰¹ may have a heteroatom such as a heterocyclic ring. Specific examples thereof include lactone-containing cyclic groups each represented by General Formulae (a2-r-1) to (a2-r-7), —SO₂-containing cyclic groups each represented by General Formulae (b5-r-1) to (b5-r-4), and other heterocyclic groups each represented by Chemical Formulae (r-hr-1) to (r-hr-16). In the formulae, * represents a bonding site that is bonded to Y¹⁰¹ in Formula (b-1).

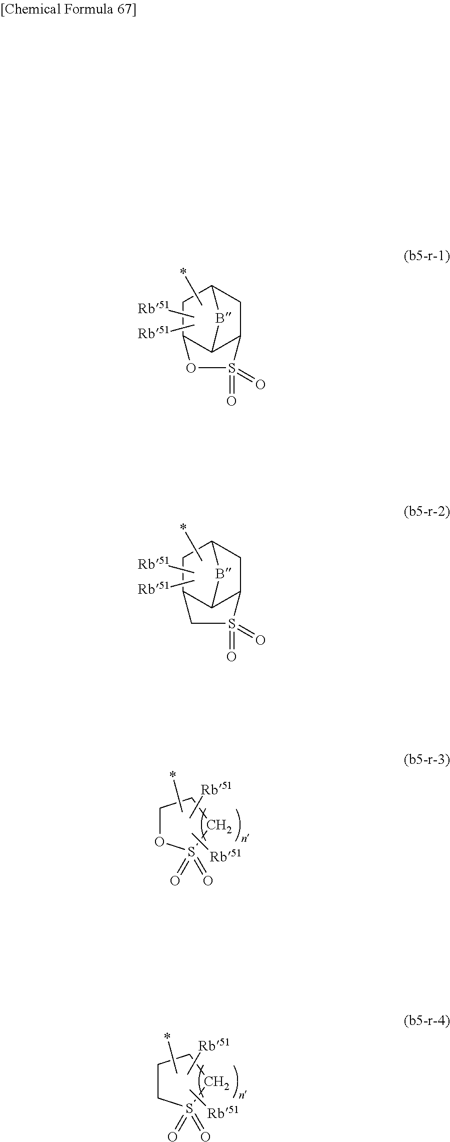

[In the formulae, Rb′⁵¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group; R″ represents a hydrogen atom, an alkyl group, a lactone-containing cyclic group, or an —SO₂-containing cyclic group; B″ represents an oxygen atom, a sulfur atom, or an alkylene group having 1 to 5 carbon atoms, which may have an oxygen atom or a sulfur atom; and n′ represents an integer of 0 to 2. * represents a bonding site.]

In General Formulae (b5-r-1) and (b5-r-2), B″ represents an alkylene group having 1 to 5 carbon atoms which may have an oxygen atom or a sulfur atom, an oxygen atom, or a sulfur atom.

B″ represents preferably an alkylene group having 1 to 5 carbon atoms or —O—, more preferably an alkylene group having 1 to 5 carbon atoms, and still more preferably a methylene group.

In General Formulae (b5-r-1) to (b5-r-4), Rb′⁵¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group, and among the examples, it is preferable that Rb′⁵¹f's each independently represent a hydrogen atom or a cyano group.

Specific examples of the groups each represented by General Formulae (b5-r-1) to (b5-1-4) are shown below. In the formulae shown below, “Ac” represents an acetyl group.

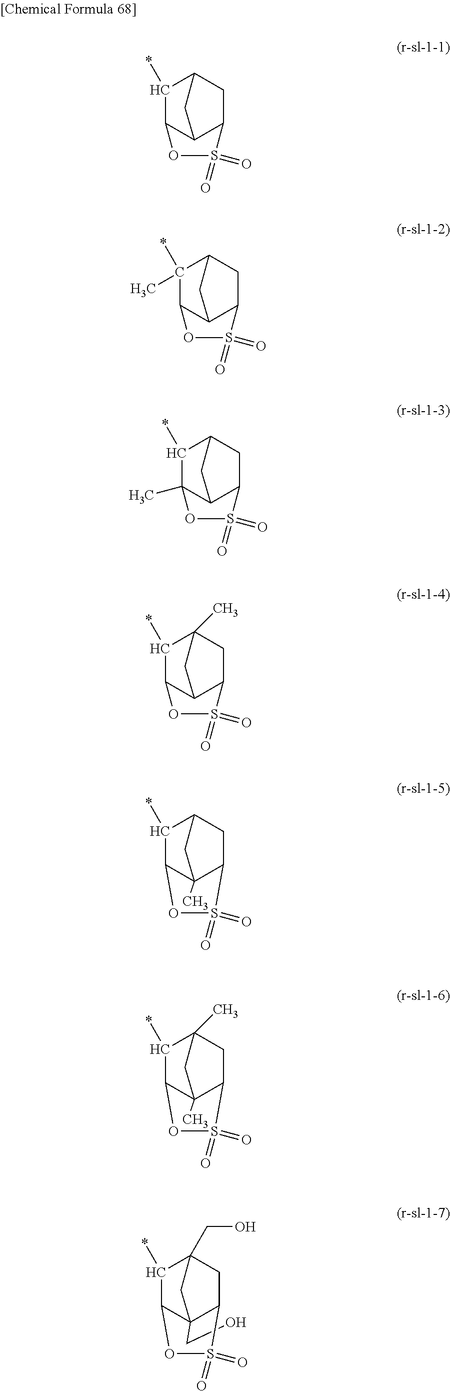

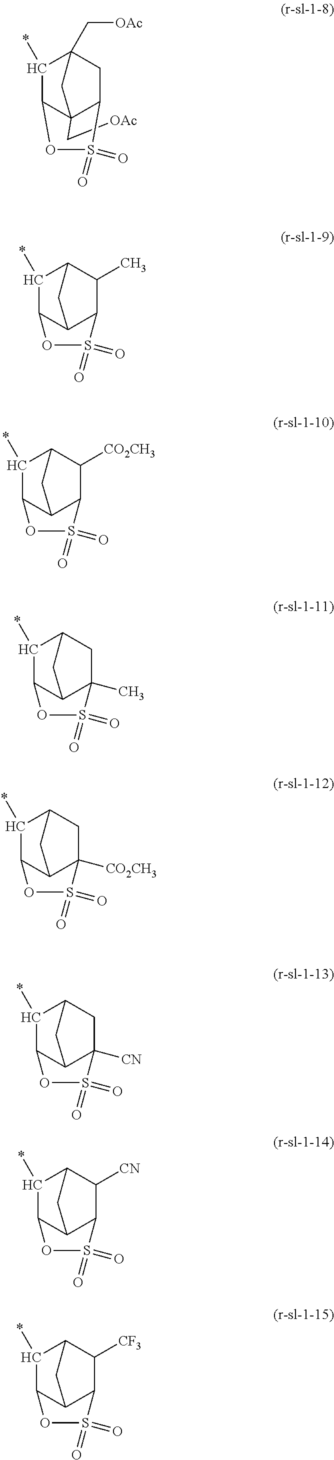

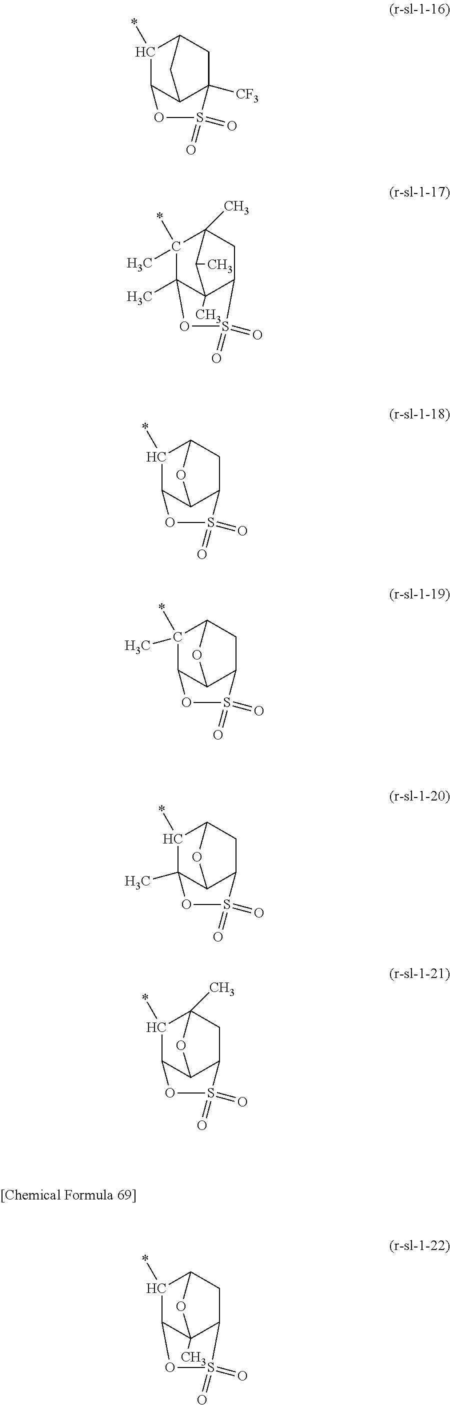

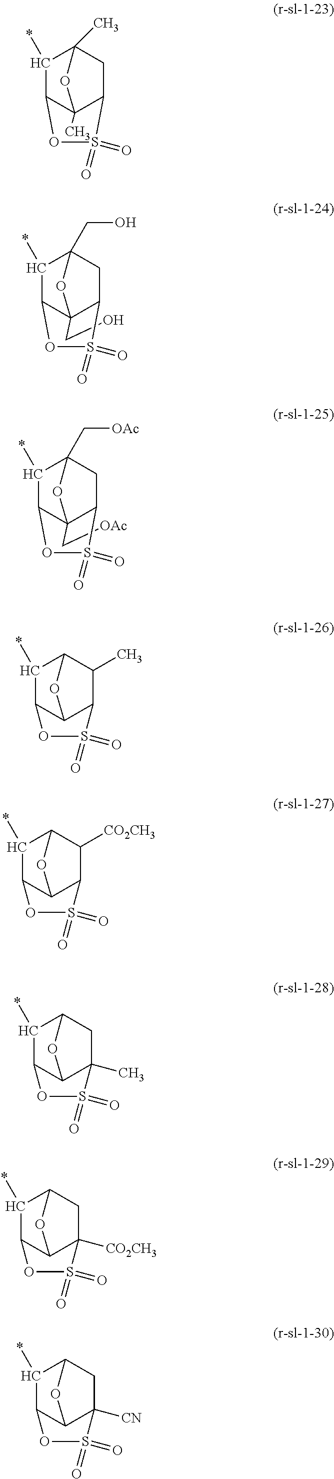

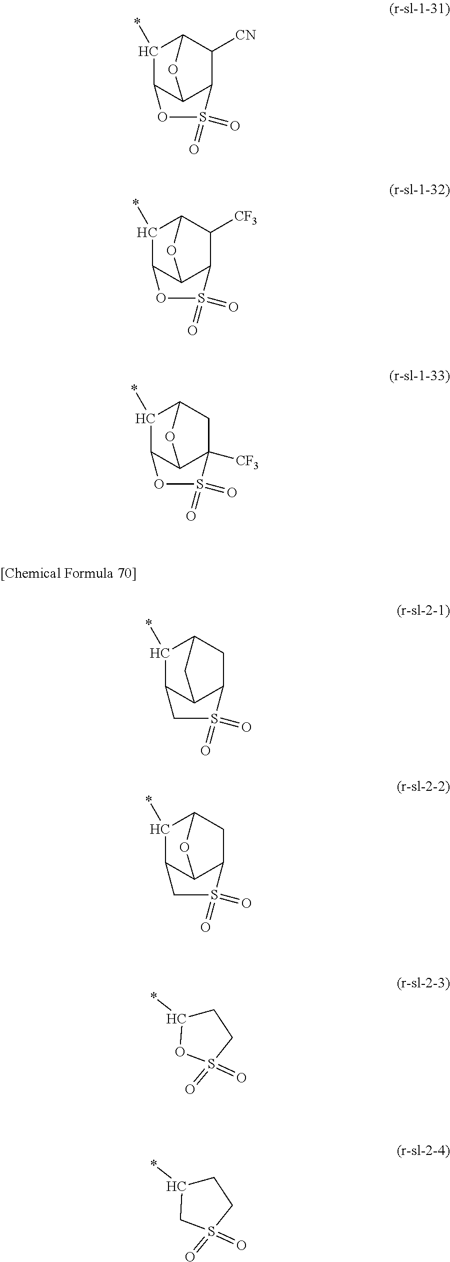

Examples of the substituent for the cyclic group as R¹⁰¹ include an alkyl group, an alkoxy group, a halogen atom, a halogenated alkyl group, a hydroxyl group, a carbonyl group, and a nitro group.

An alkyl group having 1 to 5 carbon atoms is preferable as the alkyl group serving as a substituent.

An alkoxy group having 1 to 5 carbon atoms is preferable as the alkoxy group serving as the substituent.

A fluorine atom, a bromine atom, or an iodine atom is preferable as the halogen atom serving as a substituent.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group are substituted with the above-described halogen atoms.

The carbonyl group as the substituent is a group that substitutes a methylene group (—CH₂—) constituting the cyclic hydrocarbon group.

The cyclic hydrocarbon group as R¹⁰¹ may be a condensed cyclic group containing a condensed ring in which an aliphatic hydrocarbon ring and an aromatic ring are condensed. Examples of the condensed ring include those obtained by fusing one or more aromatic rings with a polycycloalkane having a crosslinked ring polycyclic skeleton. Specific examples of the crosslinked ring polycycloalkane include a bicycloalkane such as bicyclo[2.2.1]heptane (norbornane) and bicyclo[2.2.2]octane. As the condensed cyclic group, a group having a condensed ring in which two or three aromatic rings are condensed with a bicycloalkane is preferable, and a group having a condensed ring in which two or three aromatic rings are condensed with bicyclo[2.2.2]octane is more preferable. Specific examples of the condensed cycle group as R¹⁰¹ include those represented by Formulae (r-br-1) and (r-br-2). In the formulae, * represents a bonding site that is bonded to Y¹⁰¹ in Formula (b-1).

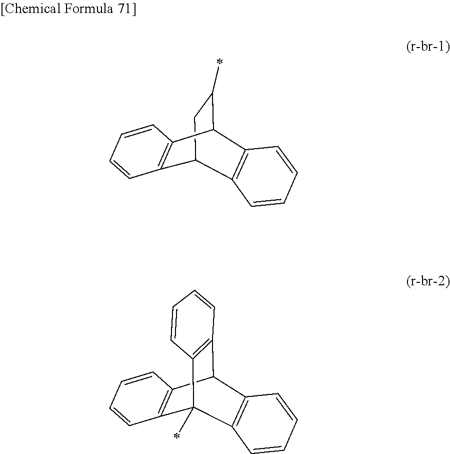

Examples of the substituent that the condensed cyclic group as R¹⁰¹ may have include an alkyl group, an alkoxy group, a halogen atom, a halogenated alkyl group, a hydroxyl group, a carbonyl group, a nitro group, an aromatic hydrocarbon group, and an alicyclic hydrocarbon group.

Examples of the alkyl group, the alkoxy group, the halogen atom, and the halogenated alkyl group as the substituent of the condensed cyclic group include those exemplified as the substituent of the cyclic group as R¹⁰¹.

Examples of the aromatic hydrocarbon group as the substituent of the condensed cyclic group include a group in which one hydrogen atom has been removed from the aromatic ring (an aryl group such as a phenyl group or a naphthyl group), a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group), and a heterocyclic group represented by any of Formulae (r-hr-1) to (r-hr-6).

Examples of the alicyclic hydrocarbon group as the substituent of the condensed cyclic group include a group in which one hydrogen atom has been removed from a monocycloalkane such as cyclopentane or cyclohexane, a group in which one hydrogen atom has been removed from a polycycloalkane such as adamantane, norbornane, isobornane, tricyclodecane, or tetracyclododecane, a lactone-containing cyclic group represented by any of General Formulae (a2-r-1) to (a2-r-7), a —SO₂-containing cyclic group represented by any of General Formulae (b5-r-1) to (b5-r-4), and a heterocyclic group represented by any of Formulae (r-hr-7) to (r-hr-16).

Chain-Like Alkyl Group which May have Substituent:

The chain-like alkyl group as R¹⁰¹ may be linear or branched.

The linear alkyl group has preferably 1 to 20 carbon atoms, more preferably 1 to carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R¹⁰¹ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (allyl group), and a butenyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R¹⁰¹ include an alkoxy group, a halogen atom, a halogenated alkyl group, a hydroxyl group, a carbonyl group, a nitro group, an amino group, and a cyclic group as R¹⁰¹.

In Formula (b-1), Y¹⁰¹ represents a single bond or a divalent linking group having an oxygen atom.

In a case where Y¹⁰¹ represents a divalent linking group having an oxygen atom, Y¹⁰¹ of may have an atom other than the oxygen atom. Examples of atoms other than an oxygen atom include a carbon atom, a hydrogen atom, a sulfur atom, and a nitrogen atom.

Examples of the divalent linking group having an oxygen atom include linking groups each represented by General Formulae (y-a1-1) to (y-a1-8). Further, in General Formulae (y-a1-1) to (y-a1-8), V′¹⁰¹ in General Formulae (y-a1-1) to (y-a1-8) is bonded to R¹⁰¹ in Formula (b-1).

In Formula (b-1), V¹⁰¹ represents a single bond, an alkylene group, or a fluorinated alkylene group. It is preferable that the alkylene group and the fluorinated alkylene group as V¹⁰¹ have 1 to 4 carbon atoms. Among these, it is preferable that V¹⁰¹ represents a single bond or a linear fluorinated alkylene group having 1 to 4 carbon atoms.

In Formula (b-1), R¹⁰² represents a fluorine atom or a fluorinated alkyl group having 1 to 5 carbon atoms. R¹⁰² represents preferably a fluorine atom or a perfluoroalkyl group having 1 to 5 carbon atoms and more preferably a fluorine atom.

In a case where Y¹⁰¹ represents a single bond, specific example of the anion moiety represented by Formula (b-1) include a fluorinated alkylsulfonate anion such as a trifluoromethanesulfonate anion or a perfluorobutanesulfonate anion. Further, in a case where Y¹⁰¹ represents a divalent linking group having an oxygen atom, specific examples thereof include an anion represented by any of Formulae (an-1) to (an-3).

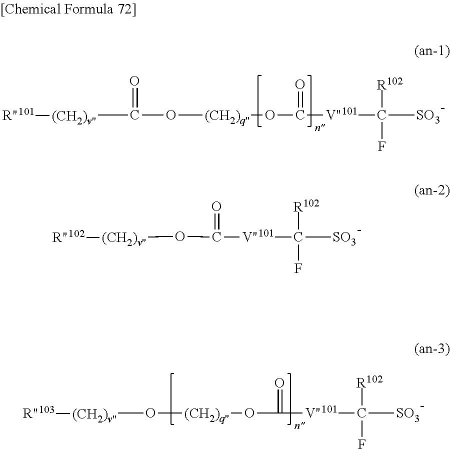

[In the formulae, R″¹⁰¹ represents an aliphatic cyclic group which may have a substituent, a monovalent heterocyclic group represented by any of Chemical Formulae (r-hr-1) to (r-hr-6), a condensed cyclic group represented by Formula (r-br-1) or (r-br-2), a chain-like alkyl group which may have a substituent, or an aromatic cyclic group which may have a substituent. R″¹⁰² represents an aliphatic cyclic group which may have a substituent, a condensed cyclic group represented by Formula (r-br-1) or (r-br-2), a lactone-containing cyclic group represented by any of General Formulae (a2-r-1) and (a2-r-3) to (a2-r-7), or a —SO₂-containing cyclic group represented by any of General Formulae (b5-r-1) to (b5-r-4). R″¹⁰³ represents an aromatic cyclic group which may have a substituent, an aliphatic cyclic group which may have a substituent, or a chain-like alkenyl group which may have a substituent. V″¹⁰¹ represents a single bond, an alkylene group having 1 to 4 carbon atoms, or a fluorinated alkylene group having 1 to 4 carbon atoms. R¹⁰² represents a fluorine atom or a fluorinated alkyl group having 1 to 5 carbon atoms. Each v″ independently represents an integer of 0 to 3, each q″ independently represents an integer of 0 to 20, and n″ represents 0 or 1.]

As the aliphatic cyclic group as R″¹⁰¹, R″¹⁰², and R″¹⁰³ which may have a substituent, the same groups as those for the cyclic aliphatic hydrocarbon group as R¹⁰¹ in Formula (b-1) are preferable. Examples of the substituent include the same groups as those for the substituent which may substitute the cyclic aliphatic hydrocarbon group as R¹⁰¹ in Formula (b-1).

As the aromatic cyclic group which may have a substituent as R″¹⁰¹ and R″¹⁰³, the groups described as the aromatic hydrocarbon group in the cyclic hydrocarbon group as R¹⁰¹ in Formula (b-1) are preferable. Examples of the substituent include the same groups as those for the substituent which may substitute the aromatic hydrocarbon group as R¹⁰¹ in Formula (b-1).

As the chain-like alkyl group as R″¹⁰¹ which may have a substituent, the same groups as those for the chain-like alkyl group as R¹⁰¹ in Formula (b-1) are preferable.

As the chain-like alkenyl group as R″¹⁰³ which may have a substituent, the same groups as those for the chain-like alkenyl group as R¹⁰¹ in Formula (b-1) are preferable.

Anions in Component (b-2)

In Formula (b-2), R¹⁰⁴ and R¹⁵ each independently represent a cyclic group which may have a substituent, a chain-like alkyl group which may have a substituent, or a chain-like alkenyl group which may have a substituent, and examples thereof include those for R¹⁰¹ in Formula (b-1). Here, R¹⁰⁴ and R¹⁰⁵ may be bonded to each other to form a ring.

R¹⁰⁴ and R¹⁰⁵ represent preferably a chain-like alkyl group which may have a substituent and more preferably a linear or branched alkyl group or a linear or branched fluorinated alkyl group.

The chain-like alkyl group has preferably 1 to 10 carbon atoms, more preferably 1 to 7 carbon atoms, and still more preferably 1 to 3 carbon atoms. It is preferable that the number of carbon atoms in the chain-like alkyl group as R¹⁰⁴ and R¹⁰⁵ decreases within the range of the number of carbon atoms because the solubility in a solvent for a resist is also satisfactory. Further, in the chain-like alkyl group as R¹⁰⁴ and R¹⁰⁵, it is preferable that the number of hydrogen atoms substituted with fluorine atoms is as large as possible because the acid strength increases and the transparency to high energy light with a wavelength of 250 nm or less or electron beams is improved. The proportion of fluorine atoms in the chain-like alkyl group, that is, the fluorination ratio is preferably in a range of 70% to 100% and more preferably in a range of 90% to 100%, and it is most preferable that the chain-like alkyl group is a perfluoroalkyl group in which all hydrogen atoms are substituted with fluorine atoms.

In Formula (b-2), V¹⁰² and V¹⁰⁹ each independently represent a single bond, an alkylene group, or a fluorinated alkylene group, and examples thereof include the same groups as those for V¹⁰¹ in Formula (b-1).

In Formula (b-2), L¹⁰¹ and L¹⁰² each independently represent a single bond or an oxygen atom.

Anions in Component (b-3)

In Formula (b-3), R¹⁰⁶ to R¹⁰⁸ each independently represent a cyclic group which may have a substituent, a chain-like alkyl group which may have a substituent, or a chain-like alkenyl group which may have a substituent, and examples thereof include those for R¹⁰¹ in Formula (b-1).

In Formula (b-3), L³⁰³ to L¹⁰⁵ each independently represent a single bond, —CO—, or —SO₂—,

Among the examples, the anion moiety of the component (B1) is preferably an anion of the component (b-1).

### {Cation Moiety}

In Formulae (b-1), (b-2), and (b-3) described above, M′^{m+} represents an m-valent onium cation. Among them, a sulfonium cation and an iodonium cation are preferable.

m represents an integer of 1 or greater.

Preferred examples of the cation moiety ((M′^{m+})_{l/m}) include the same cations as those for the organic cations represented by General Formulae (ca-1) to (ca-3). Among these, a cation represented by General Formula (ca-1) is more preferable, and cations represented by any of Formulae (ca-1-1) to (ca-1-84) are still more preferable.

In a case where the resist composition of the present embodiment contains the component (B1), the component (B1) may be used alone or in combination of two or more kinds thereof.

The content of the component (B0) is preferably 50% by mass or greater, more preferably 70% by mass or greater, and still more preferably 90% by mass or greater with respect to the total amount of the component (B) contained in the resist composition according to the present embodiment, and the component (B) may be formed of only the component (B0).

### Other Components

The resist composition of the present embodiment may further contain other components in addition to the component (A) and the component (B) described above. Examples of other components include a component (D), a component (E), a component (F), and a component(S) described below.

### <<Component (D): Base Component>

It is preferable that the resist composition of the present embodiment further contains a base component (component (D)) that traps an acid generated upon light exposure (that is, controls diffusion of the acid).

The component (D) acts as a quencher (acid diffusion control agent) which traps the acid generated in the resist composition upon light exposure.

Examples of the component (D) include a photodecomposable base (D1) having acid diffusion controllability (hereinafter, referred to as “component (D1)”) which is lost by the decomposition upon light exposure and a nitrogen-containing organic compound (D2) (hereinafter, referred to as “component (D) 2)”) which does not correspond to the component (D1).

Among these, from the viewpoint of achieving high sensitivity, suppressing a decrease in film thickness, and enhancing other lithography characteristics, it is preferable that the resist composition of the present embodiment further contains a photodecomposable base (component (D1)) that controls diffusion of an acid generated upon light exposure, is decomposed upon light exposure, and loses acid diffusion controllability. The compound described as the component (D1) below may be used as the acid generator component (component (B)) depending on the combination with other compounds.

### In Regard to Component (D1)

In a case where a resist composition containing the component (D1) is obtained, the contrast between an exposed portion and an unexposed portion of the resist film can be further improved in a case of forming a resist pattern.

The component (D1) is not particularly limited as long as the component is decomposed upon light exposure and loses acid diffusion controllability, and one or more compounds selected from the group consisting of a compound represented by General Formula (d1-1) (hereinafter, referred to as “component (d1-1)”), a compound represented by General Formula (d1-2) (hereinafter, referred to as “component (d1-2)”), and a compound represented by General Formula (d1-3) (hereinafter, referred to as “component (d1-3)”) are preferable.

Since the components (d1-1) to (d1-3) are decomposed and lose the acid diffusion controllability (basicity), the components (d1-1) to (d1-3) do not function as a quencher at the exposed portion of the resist film, but function as a quencher at the unexposed portion of the resist film,

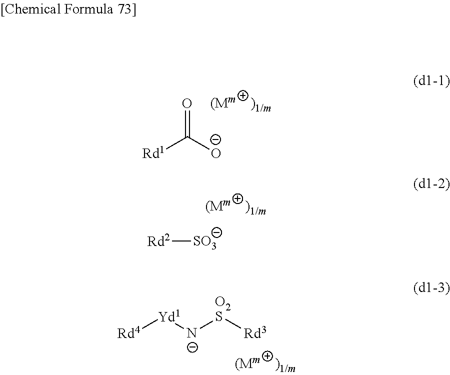

[In the formulae, Rd¹ to Rd⁴ represent a cyclic group which may have a substituent, a chain-like alkyl group which may have a substituent, or a chain-like alkenyl group which may have a substituent. Here, the carbon atom adjacent to the S atom as Rd² in Formula (d1-2) has no fluorine atom bonded thereto. Yd¹ represents a single bond or a divalent linking group. m represents an integer of 1 or greater, and each M^{m+} independently represents an m-valent organic cation.]

[Component (d1-1)]

### Anion Moiety

In Formula (d1-1), Rd¹ represents a cyclic group which may have a substituent, a chain-like alkyl group which may have a substituent, or a chain-like alkenyl group which may have a substituent, and examples thereof include the same groups as those for R′²⁰¹.

Among these, it is preferable that the group as Rd¹ represents an aromatic hydrocarbon group which may have a substituent, an aliphatic cyclic group which may have a substituent, or a chain-like alkyl group which may have a substituent. Examples of the substituent that may be included in these groups include a hydroxyl group, an oxo group, an alkyl group, an aryl group, a fluorine atom, a fluorinated alkyl group, a lactone-containing cyclic group represented by any of General Formulae (a2-r-1) to (a2-r-7), an ether bond, an ester bond, and a combination thereof. In a case where the composition has an ether bond or an ester bond as the substituent, the substituent may be present through an alkylene group, and a linking group represented by any of Formulae (y-a1-1) to (y-a1-8) is preferable as the substituent. It is noted that in a case where the aromatic hydrocarbon group, the aliphatic cyclic group, or the chain-like alkyl group, as Rd′, has a linking group represented by each of General Formulae (y-a1-1) to (y-a1-8) as a substituent, in General Formulae (y-a1-1) to (y-a1-8), the group that is bonded to a carbon atom constituting the aromatic hydrocarbon group, the aliphatic cyclic group, or the chain-like alkyl group, as Rd′, in Formula (d1-1) is V′¹⁰¹ in General Formulae (y-a1-1) to (y-a1-8).

Suitable examples of the aromatic hydrocarbon group include a phenyl group, a naphthyl group, and a polycyclic structure having a bicyclooctane skeleton (for example, a polycyclic structure formed of a bicyclooctane skeleton and a ring structure other than the bicyclooctane skeleton).

As the aliphatic cyclic group, a group in which one or more hydrogen atoms have been removed from a polycycloalkane such as adamantane, norbornane, isobornane, tricyclodecane or tetracyclododecane is more preferable.

It is preferable that the chain-like alkyl group has 1 to 10 carbon atoms, and specific examples thereof include a linear alkyl group such as a methyl group, an ethyl group, a propyl group, a butyl group, a pentyl group, a hexyl group, a heptyl group, an octyl group, a nonyl group, or a decyl group; and a branched alkyl group such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, or a 4-methylpentyl group.

In a case where the chain-like alkyl group is a fluorinated alkyl group having a fluorine atom or a fluorinated alkyl group as a substituent, the fluorinated alkyl group has preferably 1 to 11 carbon atoms, more preferably 1 to 8 carbon atoms, and still more preferably 1 to 4 carbon atoms. The fluorinated alkyl group may have an atom other than a fluorine atom. Examples of the atom other than a fluorine atom include an oxygen atom, a sulfur atom, and a nitrogen atom.

Specific preferred examples of the anion moiety in the component (d1-1) are described below.

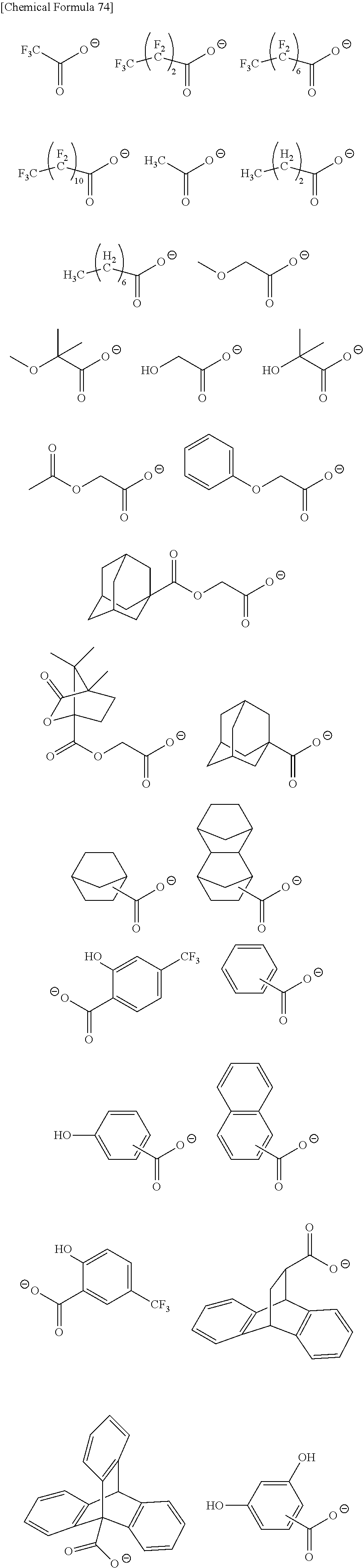

### Cation Moiety

In Formula (d1-1), M^{m+} represents an m-valent organic cation.

Suitable examples of the organic cation as M^{m+} include the same cations as those for the cations each represented by General Formulae (ca-1) to (ca-3). Among these, a cation represented by General Formula (ca-1) is more preferable, and cations each represented by Formulae (ca-1-1) to (ca-1-84) are still more preferable.

The component (d1-1) may be used alone or in combination of two or more kinds thereof.

[Component (d1-2)]

### Anion Moiety

In Formula (d1-2), Rd² represents a cyclic group which may have a substituent, a chain-like alkyl group which may have a substituent, or a chain-like alkenyl group which may have a substituent, and examples thereof include the same groups as those for R′²⁰¹.

Here, the carbon atom adjacent to the S atom in Rd² has no fluorine atom bonded thereto (the carbon atom is not substituted with a fluorine atom). In this manner, the anion of the component (d1-2) becomes an appropriately weak acid anion, thereby improving the quenching ability of the component (D).

It is preferable that Rd² represents a chain-like alkyl group which may have a substituent or an aliphatic cyclic group which may have a substituent. The chain-like alkyl group has preferably 1 to 10 carbon atoms and more preferably 3 to 10 carbon atoms. As the aliphatic cyclic group, a group in which one or more hydrogen atoms have been removed from adamantane, norbornane, isobornane, tricyclodecane, or tetracyclododecane (a group which may have a substituent); and a group in which one or more hydrogen atoms have been removed from camphor are more preferable.

The hydrocarbon group as Rd² may have a substituent, and examples of the substituent include the same groups as those for the substituent which may be included in the hydrocarbon group (such as an aromatic hydrocarbon group, an aliphatic cyclic group, or a chain-like alkyl group) as Rd¹ in Formula (d1-1).

Specific preferred examples of the anion moiety in the component (d1-2) are described below,

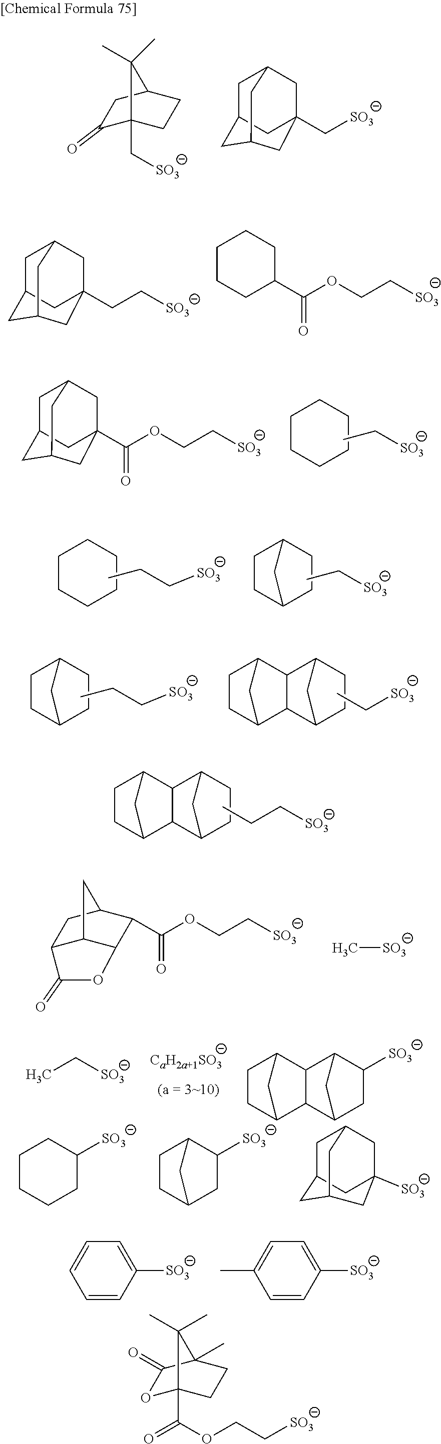

### Cation Moiety

In Formula (d1-2), M^{m+} represents an m-valent organic cation and has the same definition as that for M^{m+} in Formula (d1-1).

The component (d1-2) may be used alone or in combination of two or more kinds thereof.

[Component (d1-3)]

### Anion Moiety

In Formula (d1-3), Rd³ represents a cyclic group which may have a substituent, a chain-like alkyl group which may have a substituent, or a chain-like alkenyl group which may have a substituent, and examples thereof include the same groups as those for R′²⁰¹. Among these, a cyclic group having a fluorine atom, a chain-like alkyl group, or a chain-like alkenyl group is preferable. Among these, a fluorinated alkyl group is preferable, and the same groups as those for the fluorinated alkyl group represented by Rd¹ are more preferable.

In Formula (d1-3), Rd⁴ represents a cyclic group which may have a substituent, a chain-like alkyl group which may have a substituent, or a chain-like alkenyl group which may have a substituent, and examples thereof include the same groups as those for R²⁰¹.

Among these, an alkyl group which may have a substituent, an alkoxy group which may have a substituent, an alkenyl group which may have a substituent, or a cyclic group which may have a substituent is preferable.

It is preferable that the alkyl group as Rd⁴ is a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. Some hydrogen atoms in the alkyl group as Rd⁴ may be substituted with a hydroxyl group, a cyano group, or the like.

It is preferable that the alkoxy group as Rd⁴ is an alkoxy group having 1 to 5 carbon atoms, and specific examples of the alkoxy group having 1 to 5 carbon atoms include a methoxy group, an ethoxy group, an n-propoxy group, an iso-propoxy group, an n-butoxy group, and a tert-butoxy group. Among these, a methoxy group and an ethoxy group are preferable.

Examples of the alkenyl group as Rd⁴ include the same groups as those for the alkenyl group as R′²⁰¹. Among these, a vinyl group, a propenyl group (an allyl group), a 1-methylpropenyl group, and a 2-methylpropenyl group are preferable. These groups may have an alkyl group having 1 to 5 carbon atoms or a halogenated alkyl group having 1 to 5 carbon atoms as a substituent.

Examples of the cyclic group as Rd⁴ include the same groups as those for the cyclic group as R′²⁰¹. Among these, an alicyclic group in which one or more hydrogen atoms have been removed from a cycloalkane such as cyclopentane, cyclohexane, adamantane, norbornane, isobornane, tricyclodecane or tetracyclododecane or an aromatic group such as a phenyl group or a naphthyl group is preferable. In a case where Rd⁴ represents an alicyclic group, the resist composition is satisfactorily dissolved in an organic solvent so that the lithography characteristics are improved. Further, in a case where Rd⁴ represents an aromatic group, the resist composition has excellent light absorption efficiency in lithography using EUV or the like as an exposure light source, and thus the sensitivity and lithography characteristics are improved.

In Formula (d1-3), Yd¹ represents a single bond or a divalent linking group.

The divalent linking group as Yd¹ is not particularly limited, and examples thereof include a divalent hydrocarbon group (an aliphatic hydrocarbon group or an aromatic hydrocarbon group) which may have a substituent and a divalent linking group having a heteroatom. These divalent linking groups are the same as those for the divalent hydrocarbon group which may have a substituent and the divalent linking group having a heteroatom described in the section of the divalent linking group as Ya²¹ in Formula (a2-1).

It is preferable that Yd¹ represents a carbonyl group, an ester bond, an amide bond, an alkylene group, or a combination of these. As the alkylene group, a linear or branched alkylene group is more preferable, and a methylene group or an ethylene group is still more preferable.

Specific preferred examples of the anion moiety in the component (d1-3) are described below.

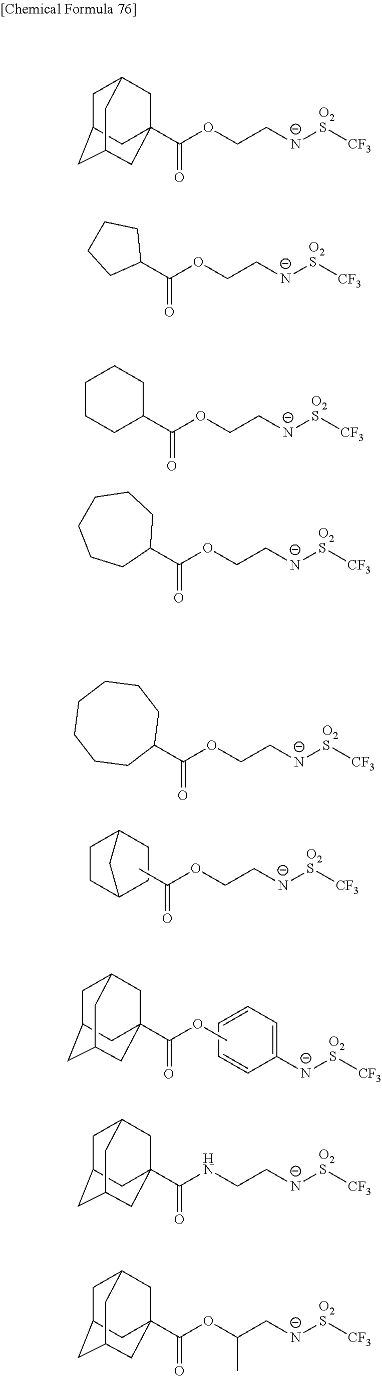

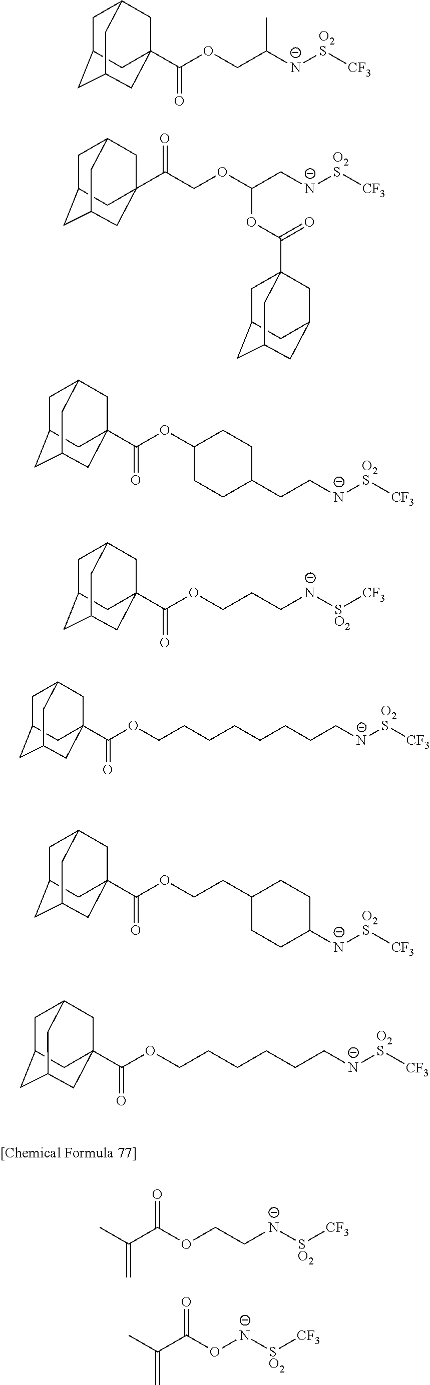

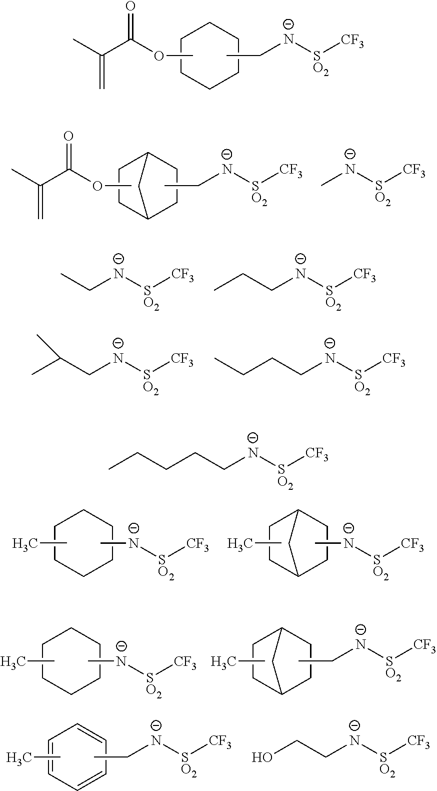

### Cation Moiety

In Formula (d1-3), M^{m+} represents an m-valent organic cation and has the same definition as that for M^{m+} in Formula (d1-1).

The component (d1-3) may be used alone or in combination of two or more kinds thereof.

As the component (D1), only one of the above-described components (d1-1) to (d1-3) or a combination of two or more kinds thereof may be used.

In a case where the resist composition contains the component (D1), the content of the component (D1) in the resist composition is preferably in a range of 0.5 to 20 parts by mass, more preferably in a range of 1 to 15 parts by mass, and still more preferably in a range of 2 to 8 parts by mass with respect to 100 parts by mass of the component (A1).

In a case where the content of the component (D1) is greater than or equal to the lower limits of the above-described ranges, particularly excellent lithography characteristics and an excellent resist pattern shape are easily obtained. On the contrary, in a case where the content is less than or equal to the upper limits of the above-described ranges, the sensitivity can be satisfactorily maintained and the throughput is also excellent.

### Method of Producing Component (D1):

The methods of producing the component (d1-1) and the component (d1-2) are not particularly limited, and these components can be produced by known methods.

Further, the method of producing the component (d1-3) is not particularly limited, and the component is produced by the same method as disclosed in United States Patent Application, Publication No. 2012-0149916.

### In Regard to Component (D2)

The component (D) may contain a nitrogen-containing organic compound component (hereinafter, referred to as “component (D2)”) that does not correspond to the component (D1) described above.

The component (D2) is not particularly limited as long as the component functions as an acid diffusion control agent and does not correspond to the component (D1), and an optional component may be selected from known components and then used. Among the examples, an aliphatic amine is preferable, and particularly a secondary aliphatic amine and a tertiary aliphatic amine are more preferable.

The aliphatic amine is an amine containing one or more aliphatic groups, and the number of carbon atoms in the aliphatic group is preferably in a range of 1 to 12.

Examples of these aliphatic amines include amines in which at least one hydrogen atom of ammonia NH₃ has been substituted with an alkyl group or hydroxyalkyl group having 12 or less carbon atoms (alkylamines or alkylalcoholamines), and cyclic amines.

Specific examples of the alkylamines and the alkylalcoholamines include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, and tri-n-dodecylamine; and alkylalcoholamines such as diethanolamine, triethanolamine, diisopropanolamine, triisopropanolamine, di-n-octanolamine, and tri-n-octanolamine. Among these, a trialkylamine having 6 to 30 carbon atoms is still more preferable, and tri-n-pentylamine or tri-n-octylamine is particularly preferable.

Examples of the cyclic amine include a heterocyclic compound having a nitrogen atom as a heteroatom. The heterocyclic compound may be a monocyclic compound (aliphatic monocyclic amine) or a polycyclic compound (aliphatic polycyclic amine).

Specific examples of the aliphatic monocyclic amine include piperidine and piperazine.

The aliphatic polycyclic amine preferably has 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl}amine, tris{2-(2-methoxyethoxy)ethyl}amine, tris{2-(2-methoxyethoxymethoxy}ethyl]amine, tris{2-(1-methoxyethoxy)ethyl}amine, tris{2-(1-ethoxyethoxy)ethyl}amine, tris{2-(1-ethoxypropoxy)ethyl}amine, tris[2-(2-(2-hydroxyethoxy)ethoxy}ethyl]amine, and triethanolamine triacetate. Among these, triethanolamine triacetate is preferable.

As the component (D2), an aromatic amine may be used.

Examples of aromatic amines include 4-dimethylaminopyridine, pyrrole, indole, pyrazole, imidazole, and derivatives thereof, tribenzylamine, 2,6-diisopropylaniline, N-tert-butoxycarbonylpyrrolidine, and 2,6-di-tert-butylpyridine.

The component (D2) may be used alone or in combination of two or more kinds thereof.

In a case where the resist composition contains the component (D2), the content of the component (D2) in the resist composition is typically in a range of 0.01 to 5 parts by mass with respect to 100 parts by mass of the component (A1). In a case where the content thereof is set to be in the above-described range, the resist pattern shape, the post exposure temporal stability, and the like are improved.

<<Component (E): At Least One Compound Selected from Group Consisting of Organic Carboxylic Acid, Phosphorus Oxo Acid, and Derivatives Thereof>>

For the purpose of preventing any deterioration in sensitivity and improving the resist pattern shape and the post exposure temporal stability, the resist composition according to the present embodiment may contain, as an optional component, at least one compound (E) (hereinafter referred to as “component (E)”) selected from the group consisting of an organic carboxylic acid, and a phosphorus oxo acid and a derivative thereof.

Specific examples of the organic carboxylic acid include acetic acid, malonic acid, citric acid, malic acid, succinic acid, benzoic acid, and salicylic acid. Among these, salicylic acid is preferable.

Examples of the phosphorus oxo acid include phosphoric acid, phosphonic acid, and phosphinic acid. Among these, phosphonic acid is particularly preferable.

Examples of the phosphorus oxo acid derivative include an ester obtained by substituting a hydrogen atom in the above-described oxo acid with a hydrocarbon group. Examples of the hydrocarbon group include an alkyl group having 1 to 5 carbon atoms and an aryl group having 6 to 15 carbon atoms.

Examples of the phosphoric acid derivatives include phosphoric acid esters such as phosphoric acid di-n-butyl ester and phosphoric acid diphenyl ester.

Examples of the phosphonic acid derivatives include phosphonic acid esters such as phosphonic acid dimethyl ester, phosphonic acid di-n-butyl ester, phenylphosphonic acid, phosphonic acid diphenyl ester, and phosphonic acid dibenzyl ester.

Examples of the phosphinic acid derivatives include phosphinic acid ester and phenylphosphinic acid.

In the resist composition of the present embodiment, the component (E) may be used alone or in combination of two or more kinds thereof.

In a case where the resist composition contains the component (E), the content of the component (E) is preferably in a range of 0.01 to 5 parts by mass and more preferably in a range of 0.05 to 3 parts by mass with respect to 100 parts by mass of the component (A). In a case where the content thereof is in the above-described range, the sensitivity, lithography characteristics, and the like are improved.

### <<Component (F): Fluorine Additive Component>>

The resist composition according to the present embodiment may further contain a fluorine additive component (hereinafter, referred to as “component (F)”) as a hydrophobic resin. The component (F) is used to impart water repellency to the resist film and used as a resin different from the component (A), whereby the lithography characteristics can be improved.

As the component (F), for example, the fluorine-containing polymer compounds described in Japanese Unexamined Patent Application, First Publication Nos. 2010-002870, 2010-032994, 2010-277043, 2011-13569, and 2011-128226 can be used.

Specific examples of the component (F) include a polymer having a constitutional unit (f1) represented by General Formula (f1-1). As the polymer, a polymer (homopolymer) formed of only the constitutional unit (f1) represented by Formula ($1-1); a copolymer of the constitutional unit (1) and the constitutional unit (a1); or a copolymer of the constitutional unit (f1), a constitutional unit derived from acrylic acid or methacrylic acid, and the constitutional unit (a1) is preferable, and a copolymer of the constitutional unit (f1) and the constitutional unit (a1) is more preferable. Here, as the constitutional unit (a1) copolymerized with the constitutional unit (f1), a constitutional unit derived from 1-ethyl-1-cyclooctyl (meth)acrylate or a constitutional unit derived from 1-methyl-1-adamantyl (meth)acrylate is preferable, and a constitutional unit derived from 1-ethyl-1-cyclooctyl (meth)acrylate is more preferable.

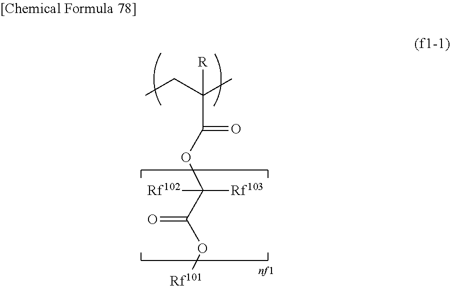

[In the formula, R has the same definition as described above, Rf¹⁰² and Rf¹⁰³ each independently represent a hydrogen atom, a halogen atom, an alkyl group having 1 to 5 carbon atoms, or a halogenated alkyl group having 1 to 5 carbon atoms, and Rf¹⁰² and Rf¹⁰³ may be the same as or different from each other. nf¹ represents an integer of 0 to 5, and Rf¹⁰¹ represents an organic group having a fluorine atom.]

In Formula (f1-1), R bonded to the carbon atom at the α-position has the same definition as described above. It is preferable that R represents a hydrogen atom or a methyl group.

In Formula (f1-1), a fluorine atom is preferable as the halogen atom as Rf¹⁰² and Rf¹⁰³. Examples of the alkyl group having 1 to 5 carbon atoms as Rf¹⁰² and Rf¹⁰³ include the same groups as those for the alkyl group having 1 to 5 carbon atoms as R. Among the examples, a methyl group or an ethyl group is preferable, Specific examples of the halogenated alkyl group having 1 to 5 carbon atoms for Rf¹⁰² and Rf¹⁰³ include groups in which some or all hydrogen atoms of an alkyl group having 1 to 5 carbon atoms have been substituted with halogen atoms. Among these, a fluorine atom is preferable as the halogen atom. Among these, Rf¹⁰² and Rf¹⁰³ represent preferably a hydrogen atom, a fluorine atom, or an alkyl group having 1 to 5 carbon atoms, more preferably a hydrogen atom, a fluorine atom, a methyl group, or an ethyl group, and still more preferably a hydrogen atom.

In Formula (f1-1), nf¹ represents an integer of 0 to 5, preferably an integer of 0 to 3, and more preferably 1 or 2.

In Formula (f1-1), Rf¹⁰¹ represents an organic group having a fluorine atom and preferably a hydrocarbon group having a fluorine atom.

The hydrocarbon group having a fluorine atom may be linear, branched, or cyclic, and the number of carbon atoms thereof is preferably in a range of 1 to 20, more preferably in a range of 1 to 15, and particularly preferably in a range of 1 to 10.

In the hydrocarbon group having a fluorine atom, preferably 25% or greater of the hydrogen atoms in the hydrocarbon group are fluorinated, more preferably 50% or greater thereof are fluorinated, and still more preferably 60% or greater thereof are fluorinated from the viewpoint of increasing the hydrophobicity of the resist film during immersion exposure.

Among examples, Rf¹⁰¹ represents more preferably a fluorinated hydrocarbon group having 1 to 6 carbon atoms and particularly preferably a trifluoromethyl group, —CH₂—CF₃, —CH₂—CF₂—CF₃, —CH(CF₃)₂, —CH₂—CH₂—CF₃, or —CH₂—CH₂—CF₂—CF₂CF₂—CF₃.

The weight-average molecular weight (Mw) (in terms of polystyrene according to gel permeation chromatography) of the component (F) is preferably in a range of 1000 to 50000, more preferably in a range of 5000 to 40000, and most preferably in a range of 10000 to 30000. In a case where the weight-average molecular weight thereof is less than or equal to the upper limits of the above-described ranges, the resist composition exhibits a satisfactory solubility in a solvent for a resist enough to be used as a resist. Meanwhile, in a case where the weight-average molecular weight thereof is greater than or equal to the lower limits of the above-described ranges, water repellency of the resist film is improved.

Further, the dispersity (Mw/Mn) of the component (F) is preferably in a range of 1.0 to 5.0, more preferably in a range of 1.0 to 3.0, and most preferably in a range of 1.0 to 2.5.

In the resist composition according to the present embodiment, the component (F) may be used alone or in combination of two or more kinds thereof.

In a case where the resist composition contains the component (F), the content of the component (F) is preferably in a range of 0.5 to 10 parts by mass and more preferably in a range of 1 to 10 parts by mass with respect to 100 parts by mass of the component (A).

### <<Component(S): Organic Solvent Component>>

The resist composition of the present embodiment can be produced by dissolving the resist materials in an organic solvent component (hereinafter, referred to as “component(S)”).

The component(S) may be any organic solvent which can dissolve each component to be used to obtain a uniform solution, and an optional organic solvent can be appropriately selected from those which have been known as solvents of a chemically amplified resist composition and then used.

In the resist composition of the present embodiment, the component(S) may be used alone or in the form of a mixed solvent of two or more kinds thereof. Among these, PGMEA, PGME, γ-butyrolactone, EL, or cyclohexanone is preferable.

Further, a mixed solvent obtained by mixing PGMEA with a polar solvent is also preferable as the component(S). The blending ratio (mass ratio) may be appropriately determined in consideration of the compatibility between PGMEA and the polar solvent.

Further, a mixed solvent of γ-butyrolactone and at least one selected from PGMEA and EL is also preferable as the component(S). In this case, as the mixing ratio, the mass ratio between the former and the latter is preferably in a range of 70:30 to 95:5.

The amount of the component(S) to be used is not particularly limited and is appropriately set to have a concentration which enables coating a substrate or the like depending on the thickness of the coated film. The component(S) is typically used in an amount such that the solid content concentration of the resist composition is set to be in a range of 0.1% to 20% by mass and preferably in a range of 0.2% to 15% by mass.

As desired, miscible additives such as additive resins, dissolution inhibitors, plasticizers, stabilizers, colorants, halation prevention agents, and dyes for improving the performance of the resist film can be added to the resist composition of the present embodiment, as appropriate.

After the resist material is dissolved in the component(S), impurities may be removed from the resist composition of the present embodiment using a porous polyimide film, a porous polyamide-imide film, or the like. For example, the resist composition may be filtered using a filter formed of a porous polyimide film, a filter formed of a porous polyamide-imide film, a filter formed of a porous polyimide film and a porous polyamide-imide film, or the like. Examples of the porous polyimide film and the porous polyamide-imide film include those described in Japanese Unexamined Patent Application, First Publication No. 2016-155121.

The resist composition of the present embodiment described above contains a specific component (B0) represented by General Formula (b0) as the acid generator component (component (B)) that generates an acid upon light exposure. In the component (B0), the cyclic alkene A is bonded to the carbonyloxy group in General Formula (b0) as an acid dissociable group to form an acid decomposable group. At least one iodine atom (1) is bonded to the cyclic alkene A. The iodine atom has high hydrophobicity and has a large absorption of EUV having a wavelength of 13.5 nm. As described above, in the present embodiment, in a case where a structure in which an iodine atom (I) is bonded to an acid dissociable group which is a moiety that dissociates from the anion moiety is employed, the anion moiety after the dissociation of the acid dissociable group is in a state of having high solubility in a developing solution, and the hydrophilicity and the hydrophobicity with respect to the developing solution can be controlled.

In the present embodiment, since the anion moiety of the component (B0) has an iodine atom (I), the hydrophobicity of the unexposed portion of the resist film is increased, and the dissolution contrast is likely to be obtained.

In addition, since the anion moiety of the component (B0) has an iodine atom (I), the absorption increases with respect to light exposure.

Further, the anion moiety of the component (B0) contains an acid dissociable group (Rpg). In this acid dissociable group (Rpg), the cyclic alkene A has a carbon-carbon unsaturated bond in the ring skeleton. Therefore, the stability of the carbocation formed by the dissociation of the cyclic alkene A by the action of an acid is likely to increase. In particular, in a case where the carbon-carbon unsaturated bond is formed between the carbon atom (C^{α}) at an adjacent position (α-position of the carbon atom (C^{A})) of the carbon atom (C^{A}) forming a ring skeleton of the cyclic alkene A, which is bonded to the carbonyloxy group in Formula (b0), and the carbon atom (C^{β}) at an adjacent position (β-position of the carbon atom (C^{A})) of the carbon atom (C^{α}), the stability of the carbocation is likely to be increased. That is, acid dissociation upon light exposure is promoted, which contributes to improvement of sensitivity.

It is presumed that, in a case where the above-described effects occur synergistically, according to the resist composition of the present embodiment, high sensitivity can be achieved in a case of forming a resist pattern, and a resist pattern having a satisfactory shape can be formed by suppressing a decrease in film thickness during development.

### (Resist Pattern Formation Method)

A resist pattern formation method according to the second aspect of the present invention is a method including a step of forming a resist film on a support using the resist composition according to the first aspect of the present invention described above, a step of exposing the resist film to light, and a step of developing the resist film exposed to light to form a resist pattern.

According to the embodiment of the resist pattern formation method, a resist pattern formation method, which is performed in the following manner is an exemplary example.

First, a support is coated with the resist composition of the present embodiment using a spinner or the like, and a bake (post applied bake (PAB)) treatment is performed under a temperature condition of 80 °C to 150 °C for 40 to 120 seconds and preferably 60 to 90 seconds to form a resist film.

Next, the selective exposure is performed on the resist film by, for example, light exposure through a mask (mask pattern) having a predetermined pattern formed thereon using an exposure apparatus such as an electron beam lithography apparatus or an ArF exposure apparatus, or direct irradiation with an electron beam for drawing without using a mask pattern, and a bake treatment (post exposure bake (PEB)) is carried out, for example, under a temperature condition of 80 °C to 150 °C for 40 to 120 seconds and preferably 60 to 90 seconds.

Next, the resist film is subjected to a developing treatment. The developing treatment is conducted using an alkali developing solution in a case of an alkali developing process and using a developing solution containing an organic solvent (organic developing solution) in a case of a solvent developing process.

After the developing treatment, it is preferable to conduct a rinse treatment. As the rinse treatment, water rinsing using pure water is preferable in a case of the alkali developing process, and rinsing using a rinse solution containing an organic solvent is preferable in a case of the solvent developing process.

In a case of the solvent developing process, after the developing treatment or the rinse treatment, the developing solution or the rinse solution attached onto the pattern may be removed by a treatment using a supercritical fluid.

After the developing treatment or the rinse treatment, drying is conducted. As desired, a bake treatment (post bake) may be conducted after the developing treatment.

The support is not particularly limited and a known support of the related art can be used, and examples thereof include a substrate for an electronic component and a substrate on which a predetermined wiring pattern has been formed. Specific examples thereof include a metal substrate such as a silicon wafer, copper, chromium, iron, or aluminum; and a glass substrate. As the materials of the wiring pattern, copper, aluminum, nickel, or gold can be used.

The wavelength to be used for light exposure is not particularly limited and the exposure can be conducted using radiation such as an ArF excimer laser, a KrF excimer laser, an F₂ excimer laser, extreme ultraviolet (EUV) rays, vacuum ultraviolet rays (VUV), electron beams (EB), X-rays, and soft X-rays.

The resist pattern formation method of the present embodiment is particularly useful in a case where the step of exposing the resist film includes an operation of exposing the resist film to extreme ultraviolet (EUV) rays or electron beams (EB).

The method of exposing the resist film to light can be general exposure (dry exposure) conducted in air or an inert gas such as nitrogen, or liquid immersion exposure (liquid immersion lithography).

The liquid immersion exposure is an exposure method in which the region between the resist film and the lens at the lowermost position of the exposure apparatus is filled with a solvent (liquid immersion medium) in advance that has a refractive index greater than the refractive index of air, and the exposure (immersion exposure) is conducted in this state.

As the liquid immersion medium, a solvent having a refractive index greater than the refractive index of air but less than the refractive index of the resist film to be exposed is preferable, and examples thereof include water, a fluorine-based inert liquid, a silicon-based solvent, and a hydrocarbon-based solvent.

As the liquid immersion medium, water is preferably used.

As the alkali developing solution used for the developing treatment in the alkali developing process, a 0.1 to 10 mass % tetramethylammonium hydroxide (TMAH) aqueous solution is an exemplary example.

The organic solvent contained in the organic developing solution used for the developing treatment in the solvent developing process may be any solvent that is capable of dissolving the component (A) (the component (A) before light exposure) and can be appropriately selected from known organic solvents. Specific examples thereof include a polar solvent such as a ketone-based solvent, an ester-based solvent, an alcohol-based solvent, a nitrile-based solvent, an amide-based solvent, an ether-based solvent, and a hydrocarbon-based solvent.

Examples of the ester-based solvent include methyl acetate, butyl acetate, ethyl acetate, isopropyl acetate, pentyl acetate, isopentyl acetate, amyl acetate, propylene glycol monomethyl ether acetate, ethylene glycol monoethyl ether acetate, diethylene glycol monobutyl ether acetate, diethylene glycol monoethyl ether acetate, ethyl-3-ethoxypropionate, 3-methoxybutyl acetate, 3-methyl-3-methoxybutyl acetate, methyl formate, ethyl formate, butyl formate, propyl formate, ethyl lactate, butyl lactate, propyl lactate, butyl butanoate, methyl 2-hydroxyisobutyrate, isoamyl acetate, isobutyl isobutyrate, and butyl propionate.

Examples of the nitrile-based solvent include acetonitrile, propionitrile, valeronitrile, and butyronitrile.

Known additives can be blended into the organic developing solution as necessary. Examples of the additive include a surfactant. The surfactant is not particularly limited, and for example, an ionic or non-ionic fluorine-based and/or silicon-based surfactant can be used.

The developing treatment can be performed according to a known developing method, and examples thereof include a method of immersing a support in a developing solution for a certain time (a dip method), a method of raising a developing solution on the surface of a support using the surface tension and maintaining the state for a certain time (a puddle method), a method of spraying a developing solution to the surface of a support (spray method), and a method of continuously ejecting a developing solution onto a support rotating at a certain rate while scanning a developing solution ejection nozzle at a certain rate (dynamic dispense method).

As the organic solvent contained in the rinse solution used for the rinse treatment after the developing treatment in the solvent developing process, a solvent that is unlikely to dissolve a resist pattern can be appropriately selected from the organic solvents described as the organic solvent used in the organic developing solution and then used. Typically, at least one solvent selected from a hydrocarbon-based solvent, a ketone-based solvent, an ester-based solvent, an alcohol-based solvent, an amide-based solvent, and an ether-based solvent is used.

These organic solvents may be used alone or in combination of two or more kinds thereof. Further, an organic solvent other than the above-described solvents and water may be mixed and used.

The rinse treatment carried out using a rinse solution (washing treatment) can be performed according to a known rinse method. Examples of the method of performing the rinse treatment include a method of continuously ejecting a rinse solution onto a support rotating at a certain rate (rotary coating method), a method of immersing a support in a rinse solution for a certain time (dip method), and a method of spraying a rinse solution to the surface of a support (spray method).

According to the resist pattern formation method of the present embodiment described above, since the above-described resist composition is used, high sensitivity can be achieved in a case of forming a resist pattern, and a decrease in film thickness during development can be suppressed.

Various materials that are used in the resist composition according to the above-described embodiment and the resist pattern formation method according to the above-described embodiment (for example, a resist solvent, a developing solution, a rinse solution, a composition for forming an antireflection film, and a composition for forming a top coat) preferably do not contain impurities such as a metal, a metal salt containing halogen, an acid, an alkali, and a component containing a sulfur atom or phosphorus atom.

Here, examples of the impurities containing metal atoms include Na, K, Ca, Fe, Cu, Mn, Mg, Al, Cr, Ni, Zn, Ag, So, Pb, Li, and salts thereof. The content of the impurities contained in these materials is preferably 200 ppb or less, more preferably 1 ppb or less, still more preferably 100 parts per trillion (ppt) or less, particularly preferably 10 ppt or less, and most preferably substantially zero (less than or equal to the detection limit of the measuring device).

### Compound

A compound according to a third aspect of the present invention is a compound represented by General Formula (b0).

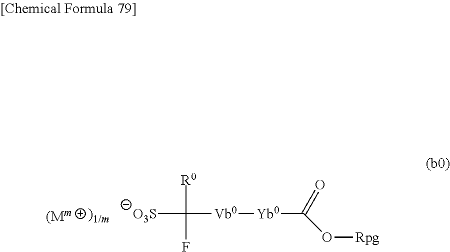

[In Formula (b0), M^{m+} represents an m-valent organic cation. m represents an integer of 1 or greater. R⁰ represents a fluorinated alkyl group having 1 to 5 carbon atoms or a fluorine atom. Vb⁰ represents an alkylene group, a fluorinated alkylene group, or a single bond. Yb⁰ represents a divalent linking group. Rpg represents an acid dissociable group represented by General Formula (a0-pg).]

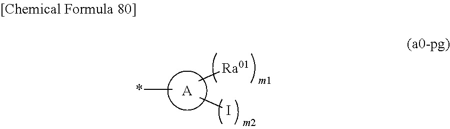

[In Formula (a0-pg), A represents a cyclic alkene having only one carbon-carbon unsaturated bond in a ring skeleton. Ra⁰¹ represents a monovalent hydrocarbon group having 1 to 20 carbon atoms. I represents an iodine atom. m1 represents an integer of 0 to 20. m2 represents an integer of 1 to 20. Here, m1+m2≤(a total number of carbon atoms constituting the ring skeleton of the cyclic alkene as A-1) is satisfied. In a case where m1 represents an integer of 2 or greater, a plurality of Ra⁰¹'s may be the same as or different from each other. * represents a bonding site that is bonded to —O— (oxy group) in —Yb⁰—CO—O—.]

The compound represented by General Formula (b0) is the same as the component (B0) in the resist composition according to the first aspect of the present invention described above.

The compound of the present embodiment is preferably at least one selected from the group consisting of a compound represented by General Formula (b0-t1) and a compound represented by General Formula (b0-s1), more preferably at least one selected from the group consisting of a compound represented by General Formula (b0-t1-1), a compound represented by General Formula (b0-s1-1), a compound represented by General Formula (b0-s1-2), and a compound represented by General Formula (b0-s1-3), still more preferably at least one selected from the group consisting of a compound represented by General Formula (b0-(1-1), a compound represented by General Formula (b0-s1-1), and a compound represented by General Formula (b0-s1-2), and particularly preferably at least one selected from the group consisting of a compound represented by General Formula (b0-t1-1) and a compound represented by General Formula (b0-s1-1).

[Method of Producing Compound Represented by General Formula (b0)]

The component (B0) can be produced by a known production method.

As an embodiment of a method of producing the component (B0), a method of producing a compound represented by General Formula (b0′) which is an example of the component (B0) is described below.

First, a compound (X-0) represented by General Formula (X-0) is obtained by reacting a compound (X-0-1) represented by General Formula (X-0-1) with a compound (Alc-1) represented by General Formula (Alc-1) which contains a predetermined acid dissociable group (Rpg) (first step).

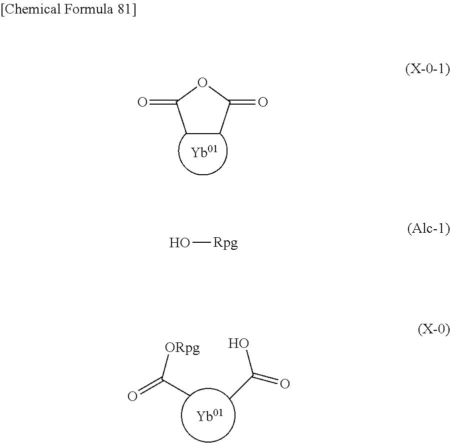

[In the formula, Yb⁰¹ represents a condensed cyclic group obtained by removing two hydrogen atoms from a condensed ring having one or more aromatic rings. Rpg represents an acid dissociable group represented by General Formula (a0-pg).]

Next, a compound (B0pre) represented by General Formula (B0pre) is obtained by reacting the compound (X-0) with a compound (S-0) represented by General Formula (S-0) (second step).

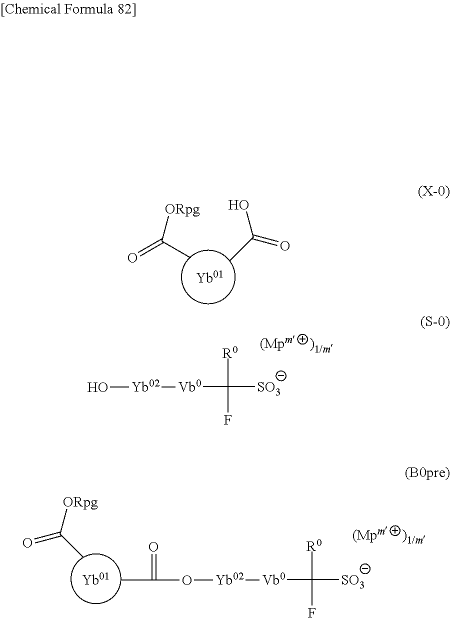

[In the formulae, Rpg represents an acid dissociable group represented by General Formula (a0-pg). Yb⁰¹ represents a condensed cyclic group obtained by removing two hydrogen atoms from a condensed ring containing one or more aromatic rings. Yb⁰² represents a divalent linking group or a single bond. Vb⁰ represents an alkylene group, a fluorinated alkylene group, or a single bond. R⁰ represents a fluorinated alkyl group having 1 to 5 carbon atoms or a fluorine atom. Mp^{m′+} represents an organic cation or a metal cation. m′ represents an integer of 1 or greater.]

Next, a target compound represented by General Formula (b0′) can be obtained by carrying out a salt exchange reaction between the compound (B0pre) and a compound (S-1) represented by General Formula (S-1) (third step).

Further, in the following reaction formulae, a moiety represented by “—Yb⁰¹—C(═O)—O—Yb⁰²—” corresponds to “Yb⁰” in General Formula (b0).

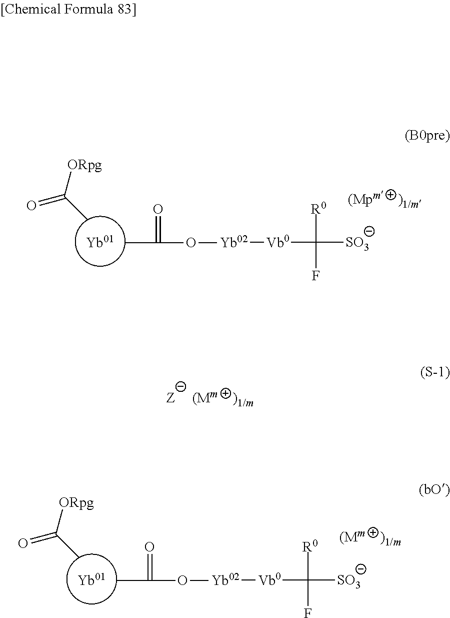

[In the formulae, Rpg represents an acid dissociable group represented by General Formula (a0-pg). Yb⁰¹ represents a condensed cyclic group obtained by removing two hydrogen atoms from a condensed ring containing one or more aromatic rings. Yb⁰² represents a divalent linking group or a single bond. Vb⁰ represents an alkylene group, a fluorinated alkylene group, or a single bond. R⁰ represents a fluorinated alkyl group having 1 to 5 carbon atoms or a fluorine atom. Mp^{m′+} represents an organic cation or a metal cation. m′ represents an integer of 1 or more. Z⁻ represents a counter anion. M^{m+} represents an m-valent organic cation. m represents an integer of 1 or greater.]

### First Step:

The first step is, for example, a step of dissolving the compound (X-0-1) and the compound (Alc-1) in an organic solvent and carrying out a reaction in the presence of a base to obtain a compound (X-0).

The organic solvent in a case of reacting the compound (X-0-1) with the compound (Alc-1) may be any solvent as long as the compound (X-0-1) and the compound (Ale-1) are dissoluble in the solvent and the solvent does not react with the compound (X-0-1) and the compound (Ale-1), and examples thereof include dichloromethane, dichloroethane, chloroform, tetrahydrofuran, N,N-dimethylformamide, acetonitrile, propionitrile, N,N′-dimethylacetamide, and dimethyl sulfoxide.

Specific examples of the base include sodium hydride, K₂CO₃, Cs₂CO₃, lithium diisopropylamide (LDA), triethylamine, and 4-dimethylaminopyridine.

In the first step, the reaction temperature is, for example, in a range of 0 °C to 50 °C, and the reaction time is, for example, 10 minutes or longer and 24 hours or shorter.

In the formula, Yb⁰ represents a condensed cyclic group obtained by removing two hydrogen atoms from a condensed ring having one or more aromatic rings, and suitable examples thereof include those described in the section of (X), such as a polycyclic aromatic cyclic group in which aromatic rings are condensed, and an aromatic ring-aliphatic hydrocarbon ring-condensed cyclic group in which an aromatic ring and an aliphatic hydrocarbon ring are condensed.

### Second Step:

In the second step, the reaction between the compound (X-0) and the compound (S-0) can be carried out, for example, by a condensation reaction in the presence of a condensing agent and a base catalyst. By this reaction, the compound (B0pre) is obtained as an intermediate.

Specific examples of the condensing agent include N,N′-dicyclohexylcarbodiimide, N,N′-diisopropylcarbodiimide (DIC), 1-ethyl-3-(3-dimethylaminopropyl) carbodiimide hydrochloride, and carbonyldiimidazole (CDI).

Specific examples of the base catalyst include tertiary amines such as trimethylamine, triethylamine, and tributylamine, aromatic amines such as pyridine, pyrrolidinopyridine, and 4-(dimethylamino)pyridine (DMAP), diazabicyclononene (DBN), and diazabicycloundecene (DBU).

In addition, the condensation reaction in the second step may be carried out in the presence of an acid catalyst.

Specific examples of the acid catalyst include diphosphorus pentoxide and methanesulfonic acid.

Examples of the reaction solvent in the second step include dichloromethane, dichloroethane, chloroform, diethyl ether, tetrahydrofuran, N,N-dimethylformamide, acetonitrile, propionitrile, N,N′-dimethylacetamide, and dimethyl sulfoxide.

The reaction time of the second step is, for example, preferably 5 minutes or longer and 24 hours or shorter and more preferably 10 minutes or longer and 12 hours or shorter.

The reaction temperature of the second step is preferably in a range of 0 °C to 50 °C, and more preferably in a range of 10 °C to 30 °C.

After the completion of the condensation reaction, the compound in the reaction solution may be isolated and purified. A known method in the related art can be used for isolation and purification, and for example, concentration, solvent extraction, distillation, crystallization, recrystallization, or chromatography can be appropriately combined and used.

### <<Compound (S-0)>>

### Anion Moiety of Compound (S-0)

The structure of the anion moiety of the compound (S-0) is selected according to the anion moiety of the target compound.

In Formula (S-0), Yb⁰² represents a divalent linking group or a single bond. Examples of the divalent linking group as Yb⁰² include a divalent linking group having an oxygen atom. Here, examples of the divalent linking group having an oxygen atom include non-hydrocarbon-based oxygen atom-containing linking groups such as an oxygen atom (ether bond: —O—), an ester bond (—C(═O)—O—), an oxycarbonyl group (—O)—C(═O)—), an amide bond (—C(═O)—NH—), a carbonyl group (—C(═O)—), and a carbonate bond (—O—C(═O)—O—); and a combination of the non-hydrocarbon-based oxygen atom-containing linking group and a divalent hydrocarbon group which may have a substituent. Further, a sulfonyl group (—SO₂—) may be further linked to the combination.

Examples of the divalent linking group having an oxygen atom include linking groups each represented by General Formulae (y-a1-1) to (y-a1-8). It is preferable that Yb⁰² represents a single bond.

In Formula (S-0), Vb⁰ and R⁰ each have the same definition as that for Vb⁰ and R⁰ in Formula (b0).

### Cation Moiety of Compound (S-0)

In General Formula (S-0), Mp^{m′+} represents an organic cation or a metal cation. m′ represents an integer of 1 or more.

Suitable examples of the organic cation as Mp^{m′+} include an organic ammonium cation having an octanol/water partition coefficient (log P_{OW}) of 4.8 or less and an onium cation having a log P_{OW} of 4.8 or less.

In the present invention, “log P_{OW}” denotes a common logarithmic value of an octanol/water partition coefficient. “log P_{OW}” is an effective parameter that can characterize the hydrophilicity/hydrophobicity of a wide range of compounds. Generally, the partition coefficient is determined by calculation regardless of an experiment, and in the present invention, the log P_{OW} shows a value calculated, for example, by CAChe Work System Pro Version 6.1. 12.33.

The hydrophobicity increases in a case where the value of log P_{OW} increases on a positive side greater than 0, and the water solubility increases in a case where the absolute value increases on a negative side. The log P_{OW} has a negative correlation with the water solubility of an organic compound and is widely used as a parameter for estimating the hydrophilicity and hydrophobicity of an organic compound.

The log P_{OW} of the organic ammonium cation as Mp^{m′} is 4.8 or less, preferably −1.0 or greater and 4.8 or less, more preferably −1.0 or greater and 3.0 or less, still more preferably −1.0 or greater and 2.0 or less, and particularly preferably −0.5 or greater and 1.5 or less.

In a case where the log P_{OW} of the organic ammonium cation is less than or equal to the upper limits of the above-described ranges, the final target product is easily produced with a higher yield. Meanwhile, in a case where the log P_{OW} thereof is greater than or equal to the lower limits of the above-described ranges, the reaction efficiency of the second step (I) is easily increased.

Examples of the organic ammonium cation as Mp^{m′+} include a cation represented by General Formula (ca-p1) and a cation represented by General Formula (ca-p2).

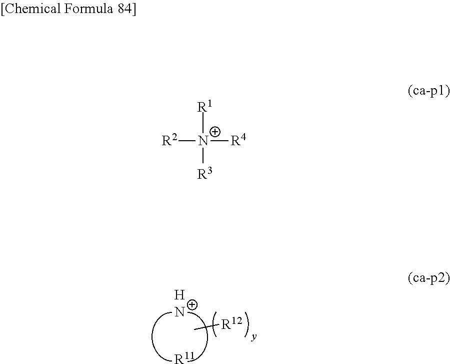

[In the formulae, R¹ to R⁴ each independently represent a hydrocarbon group which may have a substituent or a hydrogen atom. Here, at least one of R¹ to R⁴ represents a hydrocarbon group which may have a substituent. Alternatively, at least two of R¹ to R⁴ may be bonded to each other to form an alicyclic structure together with the nitrogen atom in the formula. R¹¹ represents a group that forms an aromatic ring together with a nitrogen atom to which Ru is bonded. R¹² represents an alkyl group or a halogen atom. y is an integer of 0 to 5.]

In General Formula (ca-p1), the hydrocarbon groups as R¹ to R⁴ are each independently preferably a hydrocarbon group having 1 to 15 carbon atoms and more preferably a hydrocarbon group having 1 to 10 carbon atoms. Further, the total number of carbon atoms in the hydrocarbon group as R¹ to R⁴ is preferably in a range of 1 to 20, more preferably in a range of 3 to 18, and still more preferably in a range of 4 to 15.

Examples of the hydrocarbon group as R¹ to R⁴ include a linear or branched alkyl group and a cyclic hydrocarbon group.

The linear or branched alkyl group is preferably a linear or branched alkyl group having 1 to 10 carbon atoms and more preferably a linear or branched alkyl group having 1 to 5 carbon atoms.

The cyclic hydrocarbon group may be an alicyclic hydrocarbon group or an aromatic hydrocarbon group.

The alicyclic hydrocarbon group is preferably a group obtained by removing one hydrogen atom from a monocycloalkane. The monocycloalkane has preferably 3 to 6 carbon atoms, and specific examples thereof include cyclopentane and cyclohexane. The aromatic hydrocarbon group is preferably a phenyl group or a benzyl group.

Examples of the substituent which may be included in the hydrocarbon group as R¹ to R⁴ include an alkoxy group, a hydroxyl group, an oxo group (═O), and an amino group.

In General Formula (ca-p2), R¹¹ represents a group that forms an aromatic ring together with the nitrogen atom to which the R¹¹ is bonded. The aromatic ring is preferably a 4- to 7-membered ring, more preferably a 4- to 6-membered ring, and still more preferably a 6-membered ring.

In General Formula (ca-p2), examples of the alkyl group as R¹² include the same groups as those for the linear or branched alkyl group as R¹ to R⁴.

In General Formula (ca-p2), y represents an integer of 0 to 5, preferably 0 or 1, and more preferably 0.

Hereinafter, specific examples of the organic ammonium cation represented by Mp^{m′+} as the cation moiety of the compound (S-0) will be shown. In addition, the log P_{OW} value calculated by CAChe Work System Pro Version 6.1.12.33 is shown for each organic ammonium cation.

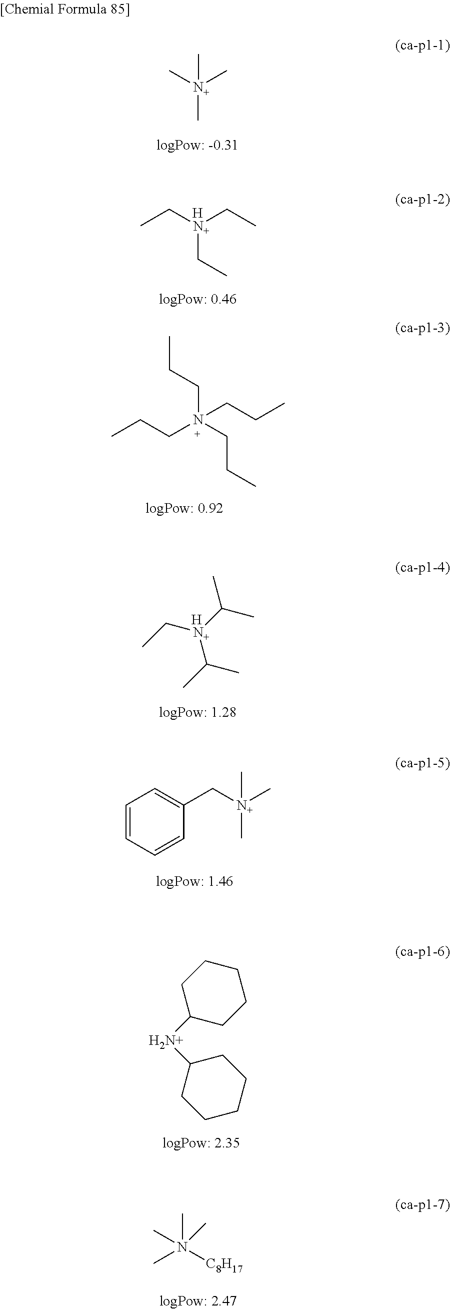

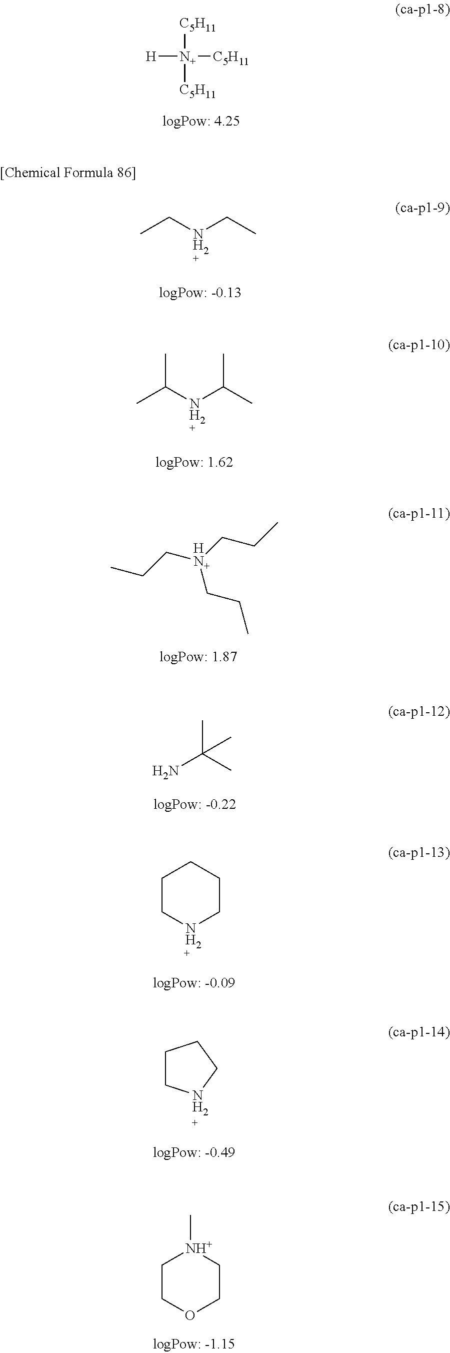

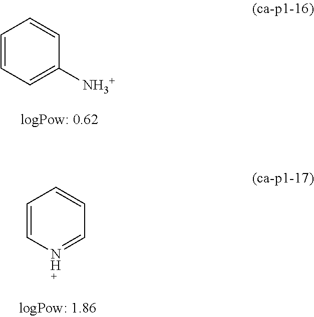 [SMILES: [NH3+]c1ccccc1 ; c1ccncc1]

The log P_{OW} of the onium cation as Mp^{m′+} is 4.8 or less, preferably −1.0 or greater and 4.8 or less, more preferably −1.0 or greater and 3.0 or less, still more preferably −1.0 or greater and 2.0 or less, and particularly preferably −0.5 or greater and 1.5 or less.

In a case where the log P_{OW} of the onium cation is less than or equal to the upper limits of the above-described ranges, the final target product is easily produced with a higher yield. Further, in a case where the log P_{OW} thereof is greater than or equal to the lower limits of the above-described ranges, the function of an acid generator for a resist composition (particularly an acid diffusion control agent) is easily exhibited.

Examples of the onium cation as Mp^{m′+} include a cation represented by General Formula (ca-p3) and a cation represented by General Formula (ca-p4).

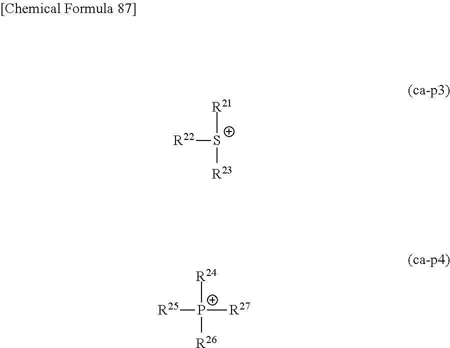

[In the formulae, R²¹ to R²³ each independently represent a hydrocarbon group which may have a substituent. R²⁴ to R²⁷ each independently represent a hydrocarbon group which may have a substituent.]

In General Formula (ca-p3), the description of the hydrocarbon group as R²¹ to R²³ and the substituent which may be contained in the hydrocarbon group is the same as the description of the hydrocarbon group as R¹ to R⁴ and the substituent which may be contained in the hydrocarbon group in General Formula (ca-p1).

The hydrocarbon group as R²¹ to R²² is preferably a linear or branched alkyl group, more preferably a linear or branched alkyl group having 1 to 10 carbon atoms, and still more preferably a linear or branched alkyl group having 1 to 5 carbon atoms.

In General Formula (ca-p4), the description of the hydrocarbon group as R²⁴ to R²⁷ and the substituent which may be contained in the hydrocarbon group is the same as the description of the hydrocarbon group as R¹ to R⁴ and the substituent which may be contained in the hydrocarbon group in General Formula (ca-p1).

The hydrocarbon group as R²⁴ to R²⁷ is preferably a linear or branched alkyl group, more preferably a linear or branched alkyl group having 1 to 10 carbon atoms, and still more preferably a linear or branched alkyl group having 1 to 5 carbon atoms.

Specific examples of the onium cation as Mp^{m′+} are shown below as the cation moiety of the compound (S-0). In addition, the log P_{OW} value calculated by CAChe Work System Pro Version 6.1.12.33 is shown for each onium cation.

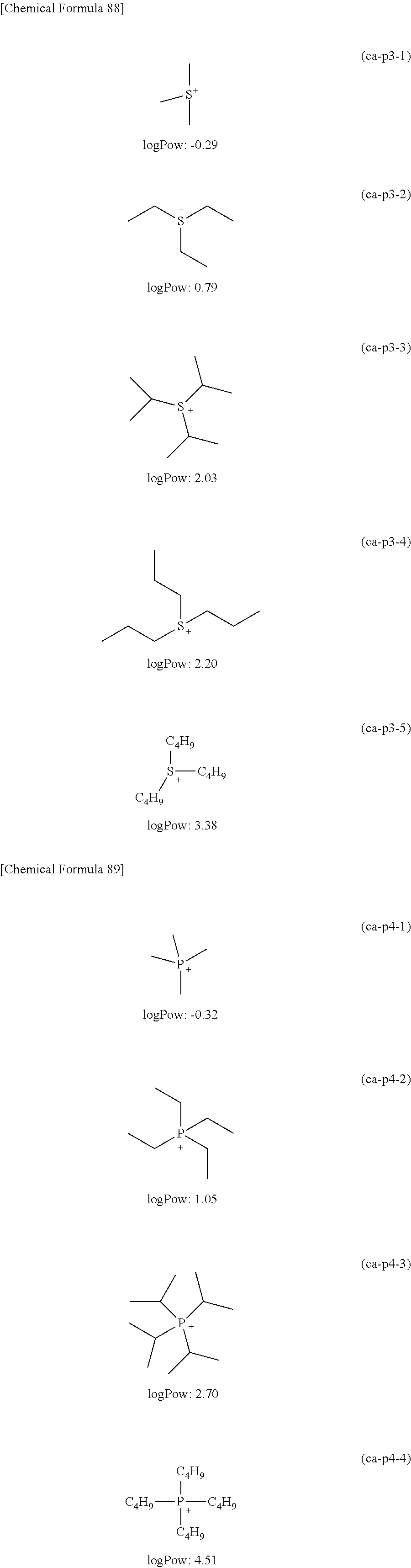

Examples of the metal cation as Mp^{m′+} include an alkali metal ion, an alkaline earth metal ion, a rubidium ion, a strontium ion, and a yttrium ion. Among these, an alkali metal ion or an alkaline earth metal ion is preferable, an alkali metal ion is more preferable, a sodium ion or a lithium ion is still more preferable, and a sodium ion is particularly preferable.

Specific examples of the compound (S-0) used in the method of producing a compound according to the present embodiment are shown below.

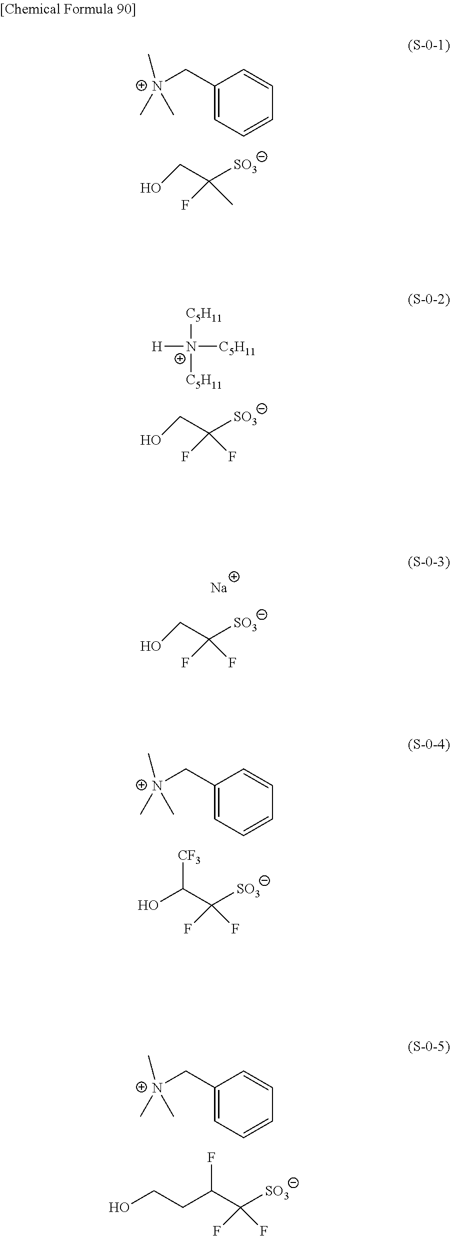

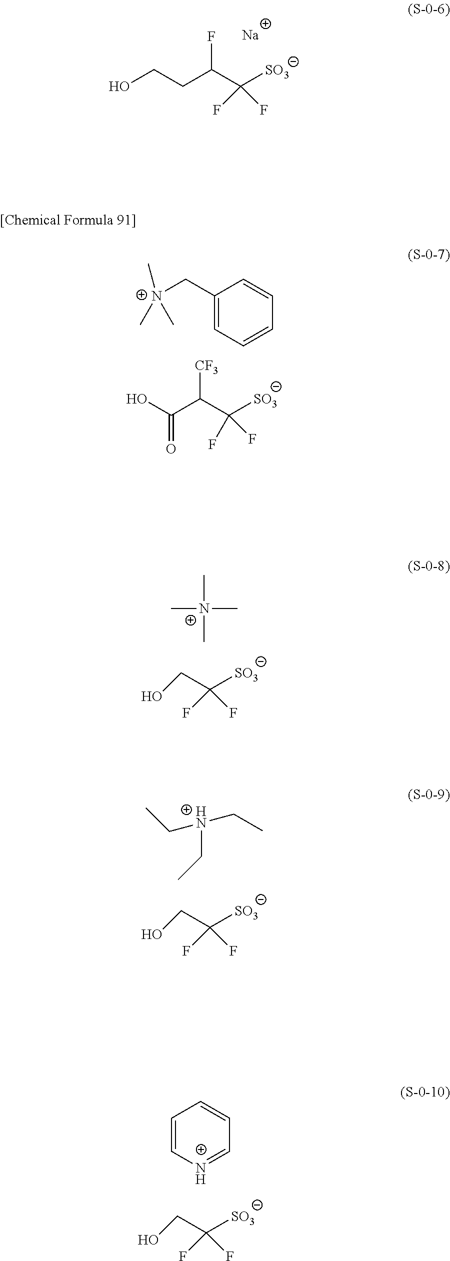

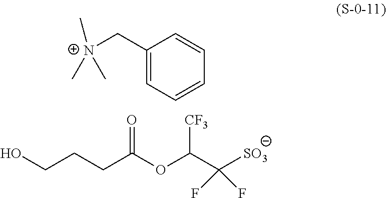

Specific examples of the compound (B0pre) as an intermediate, which is obtained in the second step described above, will be shown in the section of (Compound as intermediate) below.

### Third Step:

The third step is, for example, a step of reacting the compound (B0pre) with the compound (S-1) for salt exchange in a solvent such as water, dichloromethane, acetonitrile, or chloroform to obtain a compound represented by General Formula (b0′).

The solvent here may be an organic solvent or a mixed solvent of an organic solvent and water. Examples of the organic solvent include a ketone-based solvent such as cyclohexanone, methyl ethyl ketone, diethyl ketone, or methyl isobutyl ketone, an ether-based solvent such as diethyl ether, t-butyl methyl ether, or diisopropyl ether, a halogen-based solvent such as tetrahydrofuran, 1,3-dioxolane, dichloromethane, or 1,2-dichloroethane, an ester-based solvent such as ethyl acetate or propylene glycol monomethyl ether acetate, propionitrile, and a mixed solvent thereof.

In the third step, the reaction temperature is, for example, in a range of 0 °C to 100 °C, and the reaction time is, for example, 10 minutes or longer and 24 hours or shorter.

In the formulae, examples of the counter anion as Z′ include ions that can be an acid having a lower acidity than the intermediate represented by General Formula (B0pre), and specific examples thereof include halogen ions such as a bromine ion and a chlorine ion; BF₄⁻, AsF₆⁻, SbF₆⁻, PF₆⁻, and ClO₄⁻. Among these, Z⁻ represents preferably a halogen ion and more preferably a chlorine ion.

In the formulae, M^{m+} and m each have the same definition as that for M^{m+} and m in General Formula (b0).

After the salt exchange reaction is completed, the compound in the reaction solution may be isolated and purified. A known method in the related art can be used for isolation and purification, and for example, concentration, solvent extraction, distillation, crystallization, recrystallization, or chromatography can be appropriately combined and used.

The structure of the compound obtained as described above can be identified by general organic analysis methods such as ¹H-nuclear magnetic resonance (NMR) spectroscopy, ¹³C-NMR spectroscopy, ¹⁹F-NMR spectroscopy, infrared (IR) absorption spectroscopy, mass spectrometry (MS), elemental analysis, and X-ray crystal diffraction.

As the raw material that is used in each step, a commercially available raw material may be used, or a synthetic raw material may be used.

For example, in a case of synthesizing the compound (X-0-1), the compound (X-0-1) can be obtained by carrying out a Diels-Alder reaction between an aromatic compound (for example, anthracene) and an alkene (for example, maleic anhydride).

The compound according to the third aspect of the present invention described above is a compound useful as the acid generator component which is the component (B) in the resist composition according to the first aspect of the present invention described above.

### (Compound as Intermediate) Compound

A compound according to a fourth aspect of the present invention is a compound represented by General Formula (b0-p). The compound according to the present aspect is useful as an intermediate used in the method of producing a compound according to the third aspect described above.

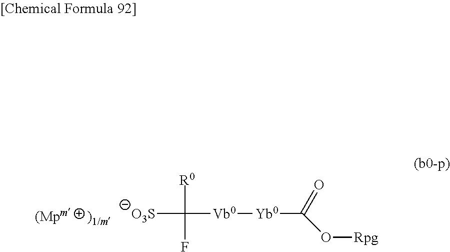

[In Formula (b0-p). Mp^{m′+} represents an organic cation or a metal cation. m′ represents an integer of 1 or greater. R⁰ represents a fluorinated alkyl group having 1 to carbon atoms or a fluorine atom. Vb⁰ represents an alkylene group, a fluorinated alkylene group, or a single bond. Yb⁰ represents a divalent linking group. Rpg represents an acid dissociable group represented by General Formula (a0-pg).]

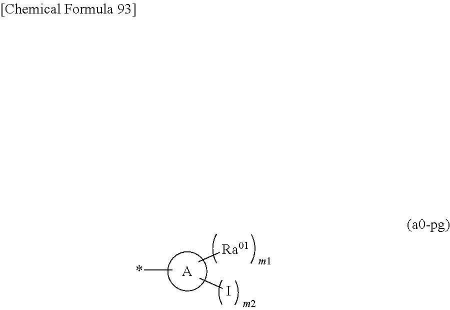

[In Formula (a0-pg), A represents a cyclic alkene having only one carbon-carbon unsaturated bond in a ring skeleton. Ra⁰¹ represents a monovalent hydrocarbon group having 1 to 20 carbon atoms. I represents an iodine atom. m1 represents an integer of 0 to 20. m2 represents an integer of 1 to 20. Here, m1+m2≤(a total number of carbon atoms constituting the ring skeleton of the cyclic alkene as A-1) is satisfied. In a case where m1 represents an integer of 2 or greater, a plurality of Ra⁰¹'s may be the same as or different from each other. * represents a bonding site that is bonded to —O— (oxy group) in —Yb⁰—CO—O—,]

In Formula (b0-p), Mp^{m′+} and m′ each have the same definition as that for Mp^{m′+} and m′ in Formula (S-0). In Formula (b0-p), Yb⁰ has the same definition as that for Yb⁰ in Formula (b0).

In Formula (b0-p), Rpg represents an acid dissociable group represented by General Formula (a0-pg) and has the same definition as that for Rpg in Formula (b0). A, Ra⁰¹, m1, and m2 in Formula (a0-pg) each have the same definition as that for A. Ra⁰¹, m1, and m2 in Rpg in Formula (b0).

Examples of one embodiment of the compound represented by General Formula (b0-p) according to the present aspect include a compound (B0pre) represented by General Formula (B0pre), which is obtained in the second step described in the section of [Method of producing compound represented by General Formula (b0)] above.

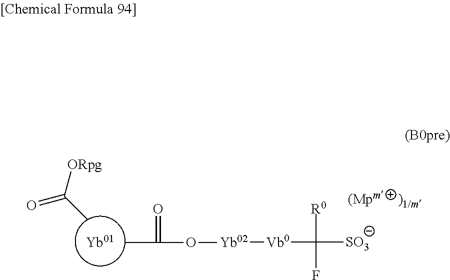

[In the formula, Rpg represents an acid dissociable group represented by General Formula (a0-pg). Yb⁰¹ represents a condensed cyclic group obtained by removing two hydrogen atoms from a condensed ring containing one or more aromatic rings. Yb⁰² represents a divalent linking group or a single bond. Vb⁰ represents an alkylene group, a fluorinated alkylene group, or a single bond. R⁰ represents a fluorinated alkyl group having 1 to 5 carbon atoms or a fluorine atom. Mp^{m′+} represents an organic cation or a metal cation. m′ represents an integer of 1 or greater.]

Rpg, Yb⁰¹, Yb⁰², Vb⁰, R⁰, Mp^{m′+}, and m′ in Formula (B0pre) each have the same definition as described above.

Specific examples of the compound (B0pre) as an intermediate are shown below, but the present invention is not limited thereto.

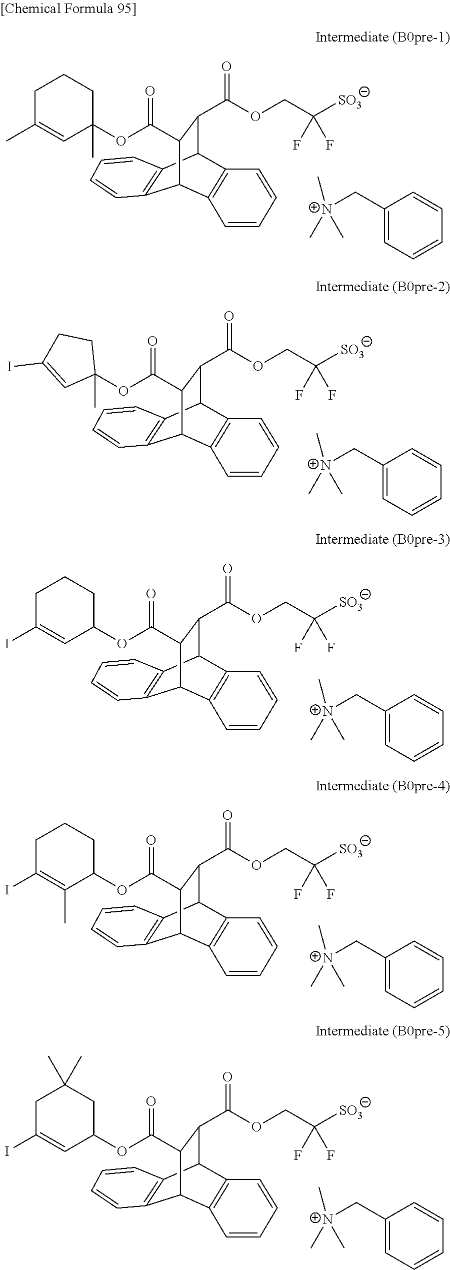

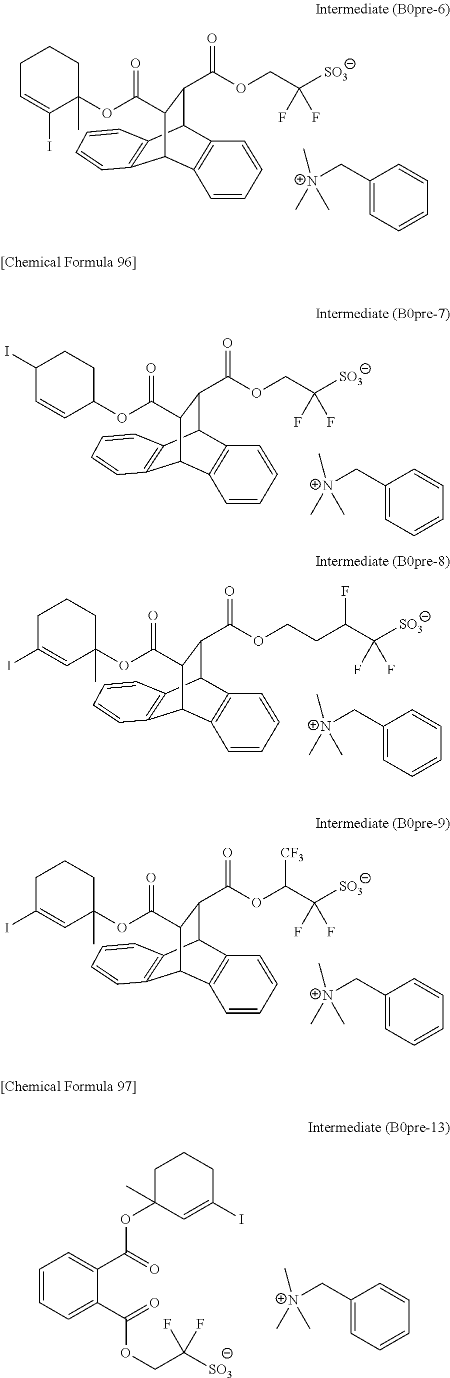

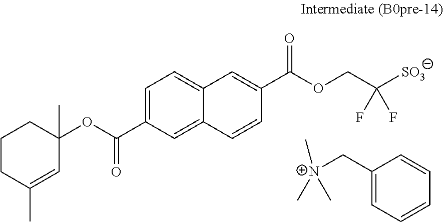

The compound according to the fourth aspect of the present invention described above is a compound useful as an intermediate in the production of the compound according to the third aspect of the present invention described above.

The intermediate of the present embodiment is a compound produced in the middle of the above-described method of producing a compound, and the yield of the compound according to the third aspect can be improved by producing the compound according to the third aspect through the intermediate of the present embodiment.

### EXAMPLES

Hereinafter, the present invention will be described in more detail based on examples, but the present invention is not limited to these examples.

In present Examples, a compound represented by Chemical Formula (X-1-1) is denoted by a “compound (X-1-1)”, and compounds represented by other chemical formulae are also denoted in the same manner.

### Production of Compound (X-1)

### Production Example 1-1

20.0 g of the compound (X-1-1), 34.5 g of 3-iodo-1-methyl-2-cyclohexen-1-ol, 4.3 g of sodium hydride (60% liquid paraffin solution), and 200.0 g of tetrahydrofuran (THE) were placed in a three-neck flask, and the mixture was stirred for 2 hours. 200.0 g of 10% hydrochloric acid was added thereto to terminate the reaction, and the mixture was extracted with dichloromethane. The organic layer was concentrated by a rotary evaporator, thereby obtaining 27.9 g of a compound (X-1).

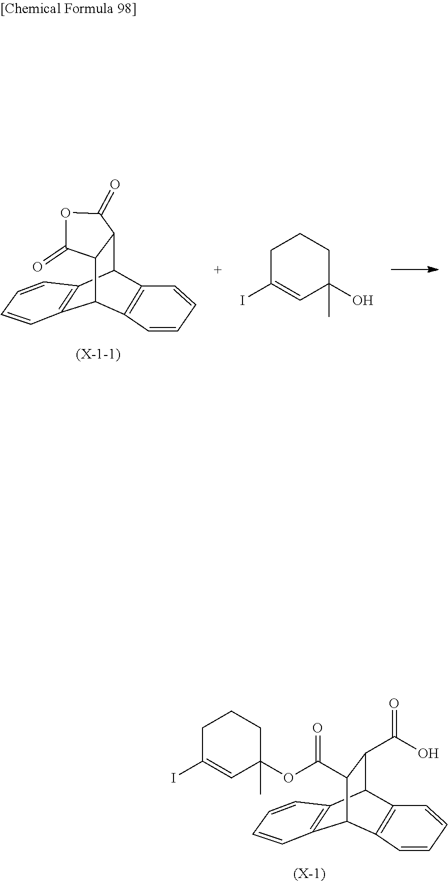

### Production Examples 1-2 to 1-4, 1-6, and 1-7

Compounds (X-2), (X-3), (X-4), (X-6), and (X-7) were obtained in the same manner as in (Production Example 1-1) except that 3-iodo-1-methyl-2-cyclohexen-1-ol in (Production Example 1-1) was changed to 3-iodo-1-methyl-2-cyclopentene-1-ol, 3-iodo-2-cyclohexen-1-ol, 3-iodo-2-methyl-2-cyclohexen-1-ol, 2-iodo-1-methyl-2-cyclohexen-1-ol, and 4-iodo-2-cyclohexen-1-ol,

### Production Example 1-5

40.0 g of 3-iodo-5,5-dimethyl-2-cyclohexen-1-one, 71.6 g of a cerium chloride heptahydrate, and 400 g of methanol were placed in a three-neck flask and stirred, and 7.8 g of sodium borohydride was added thereto in an ice bath. The mixture was stirred for 2 hours, 400 g of a saturated ammonium chloride aqueous solution and 400 g of dichloromethane were added thereto, and only the organic layer was collected. The organic layer was concentrated by a rotary evaporator, thereby obtaining 30.4 g of 3-iodo-5,5-dimethyl-2-cyclohexen-1-ol.

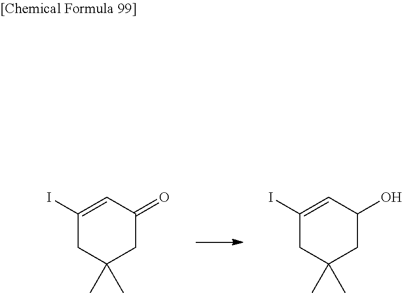

15.0 g of the compound (X-1-1), 27.4 g of 3-iodo-5,5-dimethyl-2-cyclohexen-1-ol, 3.3 g of sodium hydride (60% liquid paraffin solution), and 200.0 g of THE were placed in a three-neck flask and stirred for 2 hours. 150.0 g of 10% hydrochloric acid was added thereto to terminate the reaction, and the mixture was extracted with dichloromethane. The organic layer was concentrated by a rotary evaporator, thereby obtaining 22.1 g of a compound (X-5).

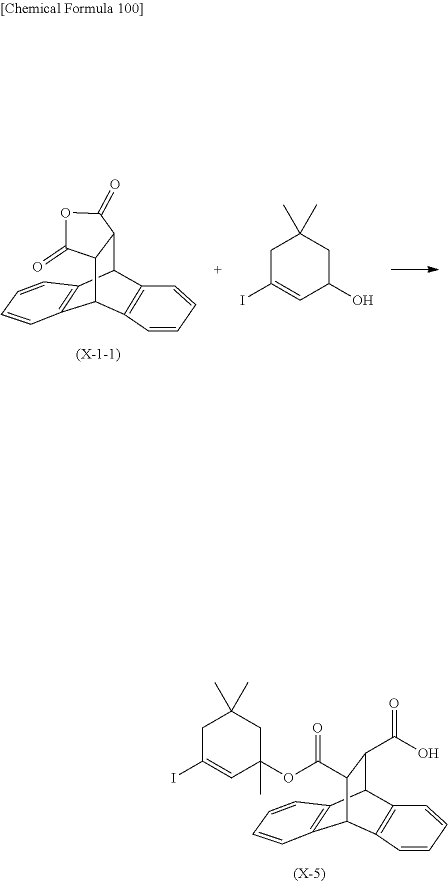

### Production Example 1-8

20.0 g of phthalic anhydride, 32.1 g of 3-iodo-1-methyl-2-cyclohexen-1-ol, 8.1 g of sodium hydride (60% liquid paraffin solution), and 200.0 g of THE were placed in a three-neck flask and stirred for 2 hours. 200.0 g of 10% hydrochloric acid was added thereto to terminate the reaction, and the mixture was extracted with dichloromethane. The organic layer was concentrated by a rotary evaporator, thereby obtaining 37.5 g of a compound (X-13).

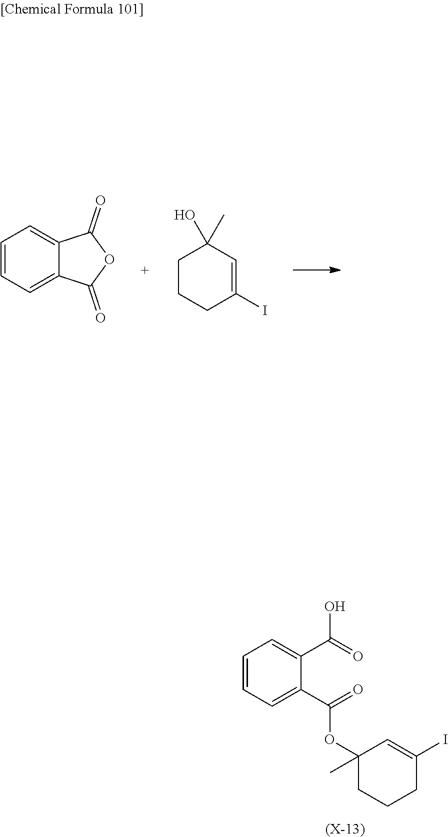

### Production Example 1-9

20.0 g of 2,6-naphthalenedicarboxylic acid, 200 g of N,N-dimethylformamide, and 33.0 g of thionyl chloride were added to a three-neck flask and stirred for 3 hours, thereby obtaining a solution of a compound (X-14-1).

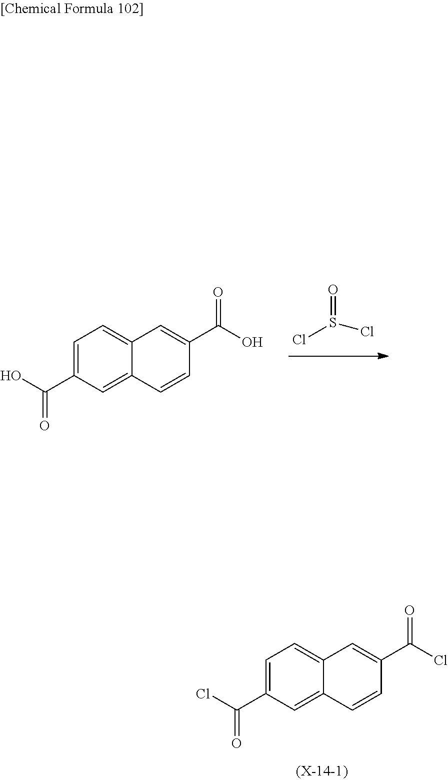

22.0 g of 3-iodo-1-methyl-2-cyclohexen-1-ol and 18.7 g of triethylamine were dissolved in 220.2 g of THE in advance, and the solution was slowly added dropwise to the solution of the compound (X-14-1). After completion of the dropwise addition, the mixture was aged for 3 hours, 440.0 g of water was added thereto, and the reaction was stopped. The reaction solution was filtered, and the obtained crystals were recrystallized with methanol, thereby obtaining 33.1 g of a compound (X-14).

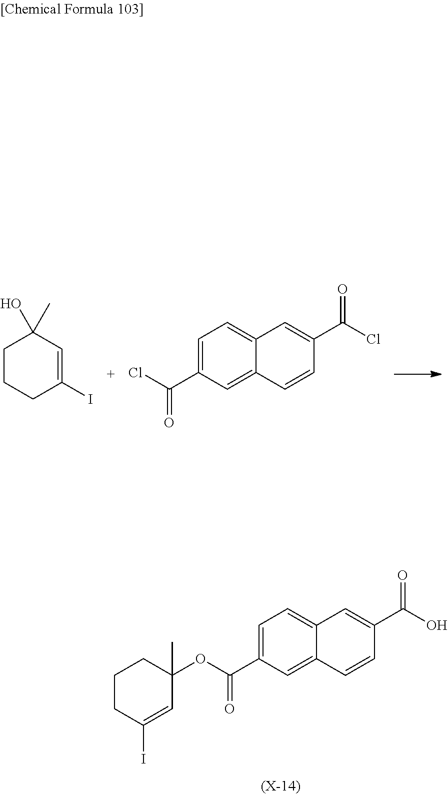

### Production of Intermediate (B0pre)

### Production Example 2-1

25.0 g of the compound (X-1), 19.7 g of the compound (S-0-1), 0.8 g of 4-dimethylaminopyridine, 9.2 g of N,N′-diisopropylcarbodiimide, and 250 g of dichloromethane were placed in a three-neck flask, and the mixture was stirred for 3 hours to prepare a reaction solution. The reaction solution was filtered, 250 g of ion exchange water was added to the filtrate, and the solution was stirred. The organic layer was concentrated with a rotary evaporator, thereby obtaining 31.0 g of an intermediate (B0pre-1).

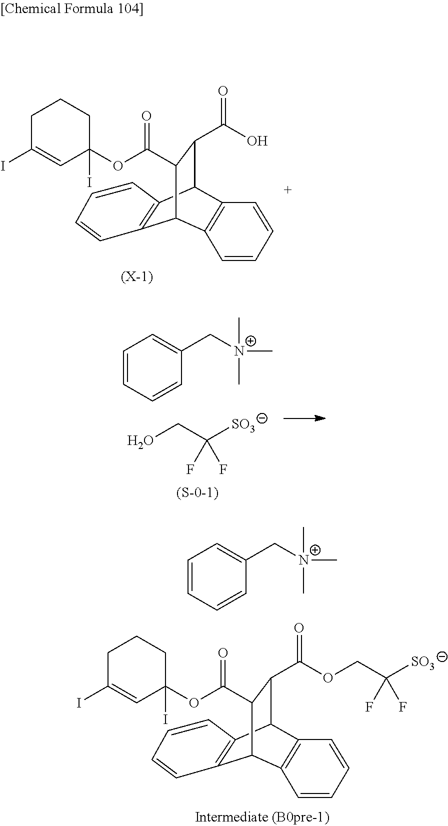

### Production Examples 2-2 to 2-14

Intermediates (B0pre-2) to (B0pre-14) were obtained in the same manner as in (Production Example 2-1) except that the combination of the compound (X-1) and the compound (S-0-1) in (Production Example 2-1) was changed to the combination shown below.

### Production Example 2-2: Combination of Compound (X-2) and Compound (S-0-1)

### Production Example 2-3: Combination of Compound (X-3) and Compound (S-0-1)

### Production Example 2-4: Combination of Compound (X-4) and Compound (S-1)

### Production Example 2-5: Combination of Compound (X-5) and Compound (S-0-1)

### Production Example 2-6: Combination of Compound (X-6) and Compound (S-0-1)

### Production Example 2-7: Combination of Compound (X-7) and Compound (S-0-1)

### Production Example 2-8: Combination of Compound (X-1) and Compound (S-0-5)

### Production Example 2-9: Combination of Compound (X-1) and Compound (S-0-4)

### Production Example 2-10: Combination of Compound (X-1) and Compound (S-0-1)

### Production Example 2-11: Combination of Compound (X-1) and Compound (S-0-1)

### Production Example 1-12: Combination of Compound (X-1) and Compound (S-0-1)

### Production Example 2-13: Combination of Compound (X-13) and Compound (S-0-1)

### Production Example 2-14: Combination of Compound (X-14) and Compound (S-0-1)

The structures of the compound (S-0-1), the compound (S-0-4), and the compound (S-0-5) are shown below,

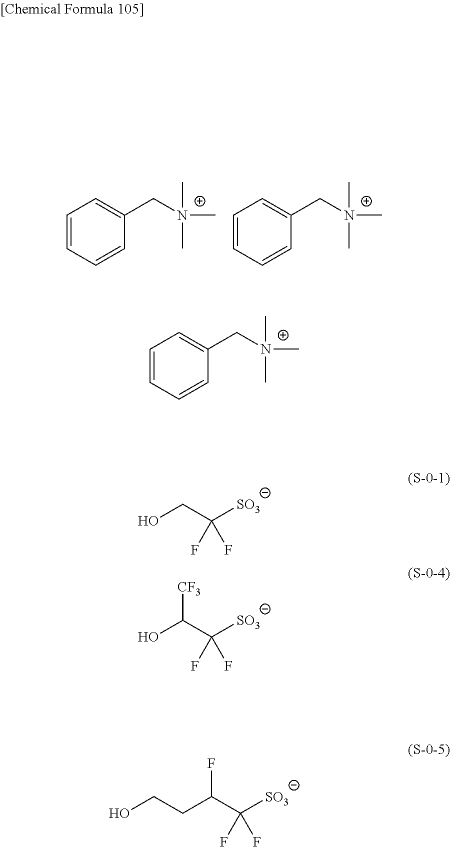

The structures of the intermediate (B0pre-1) to the intermediate (B0pre-14) are shown below.

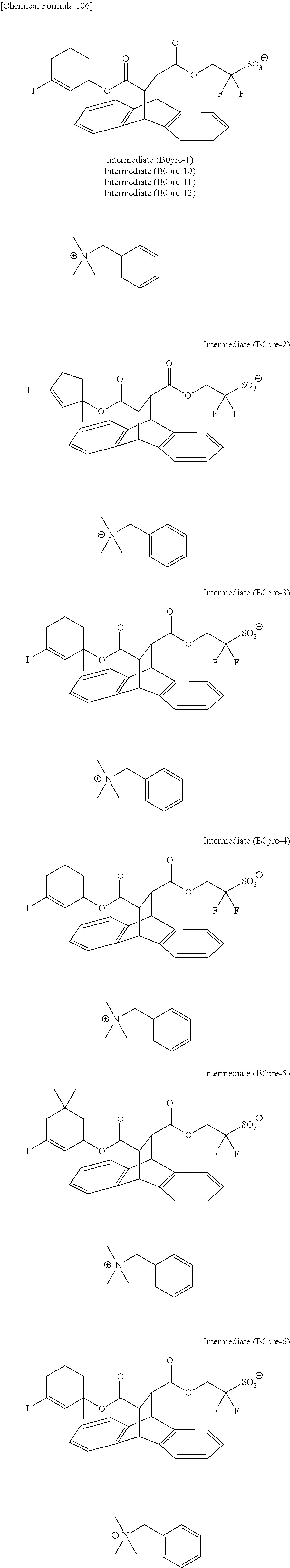

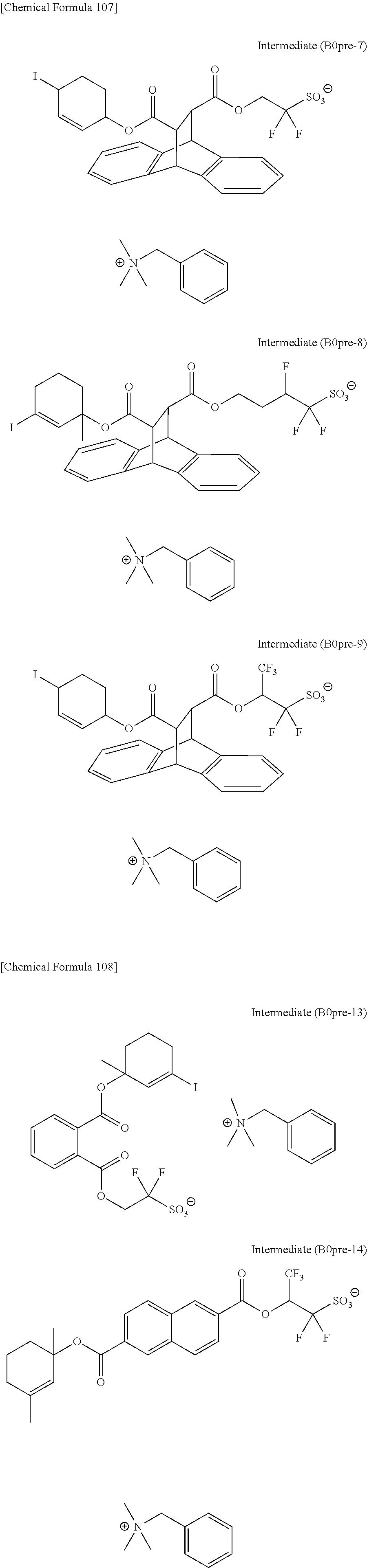

Further, the structures of the above-described intermediates (B0pre-1) to (B0pre-14) are identified from the analysis results of the ¹H-NMR measurement.

The analysis results of the structure of the intermediate (B0pre-1) by the ¹H-NMR measurement are shown below.

¹H-NMR (DMSO, 400 MHz): δ(ppm)=1.24 (3H, s), 1.65 (2H, m), 1.84 (2H, m), 1.96 (2H, m), 3.44 (2H, m), 4.44 (4H, m), 6.40 (1H, s), 7.25 (8H, m), 3.30 (9H, s), 3.90 (2H, t), 4.24 (1H, s), 4.50 (2H, s). 7.20 (5H, m)

### Production of Compound (B0)

### Production Example 3-1

25.0 g of the intermediate (B0pre-1). 9.2 g of the compound (S-1-1) for salt exchange, 250 g of dichloromethane, and 92 g of ion exchange water were placed in a three-neck flask and stirred for 3 hours. The organic layer was washed with ion exchange water and recovered. The organic layer was concentrated by a rotary evaporator, thereby obtaining 20.0 g of a target compound (B0-1).

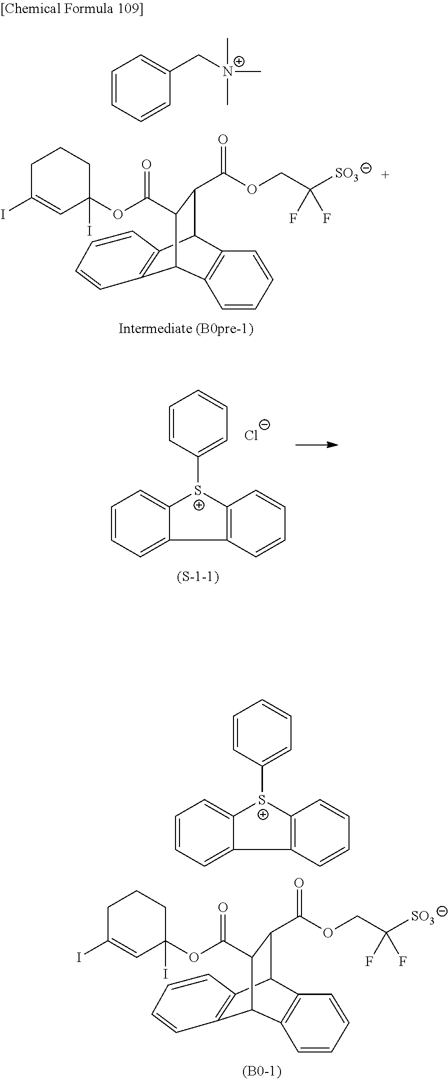

### Production Examples 3-2 to 3-14

The following compounds (B0-2) to (B0-14) were obtained in the same manner as in (Production Example 3-1) except that the combination of the intermediate (B0pre-1) and the compound (S-1-1) for salt exchange in (Production Example 3-1) was changed to a combination of the intermediates (B0pre-2) to (B0pre-14) and predetermined compounds (S-1-1) to (S-1-4) for salt exchange.

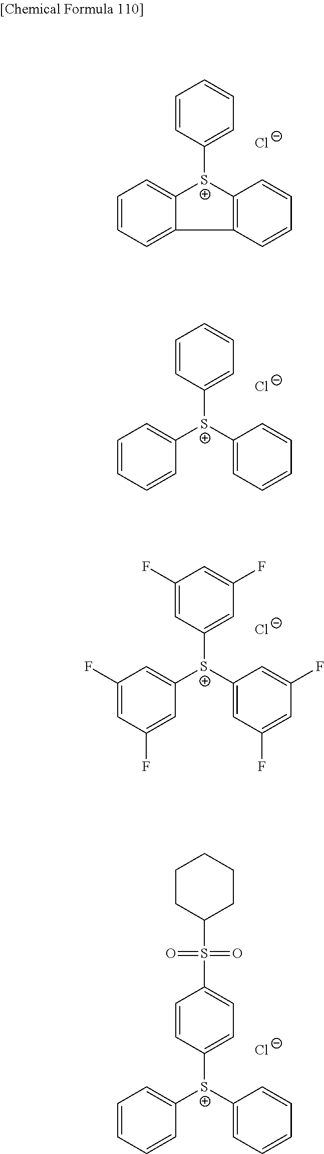

The structures of the compound (B0-1) to the compound (B0-14) are shown below.

[chemical structure]

Further, the structures of the above-described compounds (B0-1) to (B0-14) were identified from the analysis results of the ¹H-NMR measurement shown below.

Compound (B0-1): combination of precursor (B0pre-1) and compound (S-1-1) for salt exchange

¹H-NMR (DMSO, 400 MHz): δ(ppm)=1.24 (3H, s), 1.65 (2H, m), 1.84 (2H, m), 1.96 (2H, m), 3.44 (2H, m), 4.44 (4H, m), 6.40 (1H, s), 7.25 (8H, m), 7.35 (5H, m), 7.49 (6H, m), 7.77 (2H, m)

### Compound (B0-2): Combination of Precursor (B0pre-2) and Compound (S-1-1) For Salt Exchange

¹H-NMR (DMSO, 400 MHz): δ(ppm)=1.24 (3H, s), 2.09 (2H, m), 2.28 (2H, m), 3.44 (2H, m), 4.44 (4H, m), 6.41 (1H, s), 7.25 (8H, m), 7.35 (5H, m), 7.49 (6H, m), 7.77 (2H, m)

### Compound (B0-3): Combination of Precursor (B0pre-3) and Compound (S-1-1) For Salt Exchange

¹H-NMR (DMSO, 400 MHz): δ(ppm)=1.65 (2H, m), 1.89 (2H, m), 1.96 (2H, m), 3.44 (2H, m), 4.44 (4H, m), 5.13 (1H, dd). 6.40 (1H, d), 7.25 (8H, m), 7.35 (5H, m), 7.49 (6H, m), 7.77 (2H, m)

### Compound (B0-4): Combination of Precursor (B0pre-4) and Compound (S-1-1) For Salt Exchange

¹H-NMR (DMSO, 400 MHz): δ(ppm)=1.65 (2H, m), 1.79 (3H, s), 1.89 (2H, m), 1.96 (2H, m), 3.44 (2H, m), 4.44 (4H, m), 5.13 (1H, dd), 7.25 (8H, m), 7.35 (5H, m), 7.49 (6H, m), 7.77 (2H, m)

### Compound (B0-5): Combination of Precursor (B0pre-5) and Compound (S-1-1) For Salt Exchange

¹H-NMR (DMSO, 400 MHz): δ(ppm)=0.94 (6H, s), 1.78 (2H, m), 1.85 (2H, m), 3.44 (2H, m), 4.44 (4H, m), 5.13 (1H, dd), 6.40 (1H, d), 7.25 (8H, m), 7.35 (5H, m), 7.49 (6H, m), 7.77 (2H, m)

### Compound (B0-6): Combination of Precursor (B0pre-6) and Compound (S-1-1) For Salt Exchange

¹H-NMR (DMSO, 400 MHz): δ(ppm)=1.24 (3H, s), 1.65 (2H, m), 1.83 (2H, m), 2.06 (2H, m), 3.44 (2H, m), 4.44 (4H, m), 6.40 (1H, dd), 7.25 (8H, m), 7.35 (5H, m). 7.49 (6H, m), 7.77 (2H, m)

### Compound (B0-7): Combination of Precursor (B0pre-7) and Compound (S-1-1) For Salt Exchange

¹H-NMR (DMSO, 400 MHz): δ(ppm)=1.89 (2H, m), 2.20 (2H, m), 3.44 (2H, m), 4.44 (4H, m), 4.52 (1H, m), 5.13 (1H, dd), 5.59 (2H, m), 7.25 (8H, m), 7.35 (5H, m), 7.49 (6H, m), 7.77 (2H, m)

### Compound (B0-8): Combination of Precursor (B0pre-8) and Compound (S-1-1) For Salt Exchange

¹H-NMR (DMSO, 400 MHz): δ(ppm)=1.24 (3H, s), 1.65 (2H, m), 1.84 (2H, m), 1.96 (2H, m), 3.44 (2H, m), 4.06 (2H, 1), 4.44 (2H, m), 4.60 (1H, m), 6.40 (1H, s), 7.25 (8H, m), 7.35 (5H, m), 7.49 (6H, m), 7.77 (2H, m)

### Compound (B0-9): Combination of Precursor (B0pre-9) and Compound (S-1-1) For Salt Exchange

¹H-NMR (DMSO, 400 MHz): δ(ppm)=1.24 (3H, s), 1.65 (2H, m), 1.84 (2H, m), 1.96 (2H, m), 3.44 (2H, m), 4.44 (2H, m), 6.40 (1H, s), 7.25 (8H, m), 8.20 (1H, m), 7.35 (5H, m), 7.49 (6H, m), 7.77 (2H, m)

### Compound (B0-10): Combination of Precursor (B0pre-10) and Compound (S-1-2) for Salt Exchange

¹H-NMR (DMSO), 400 MHz): δ(ppm)=1.24 (3H, s), 1.65 (2H, m), 1.84 (2H, m), 1.96 (2H, m), 3.44 (2H, m), 4.44 (4H, m), 6.40 (1H, s), 7.25 (8H, m), 7.36 (15H, m)

### Compound (B0-11): Combination of Precursor (B0pre-11) and Compound (S-1-3) for Salt Exchange

¹H-NMR (DMSO, 400 MHz): δ(ppm)=1.24 (3H, s), 1.65 (2H, m), 1.84 (2H, m), 1.96 (2H, m), 3.44 (2H, m), 4.44 (4H, m), 6.40 (1H, s), 6.78 (9H, m), 7.25 (8H, m)

### Compound (B0-12): Combination of Precursor (B0pre-12) and Salt Exchange Compound (S-1-4)

¹H-NMR (DMSO), 400 MHz): δ(ppm)=1.24 (3H, s), 1.45 (2H, m), 1.48 (4H, m), 1.65 (2H, m), 1.84 (2H, m), 1.96 (2H, m), 1.97 (4H, m), 2.94 (1H, m), 3.44 (2H, m), 4.44 (4H, m), 6.40 (1H, s), 7.25 (8H, m), 7.35 (10H, m), 7.68 (2H, d), 7.80 (2H, d)

### Compound (B0-13): Combination of Precursor (B0pre-13) and Compound (S-1-1) for Salt Exchange

¹H-NMR (DMSO, 400 MHz): δ(ppm)=1.24 (3H, s), 1.65 (2H, m), 1.84 (2H, m), 1.96 (2H, m), 4.60 (2H, 1), 6.47 (1H, d), 7.35 (5H, m), 7.49 (6H, m), 7.71 (2H, m), 7.77 (2H, m), 8.04 (2H, m)

### Compound (B0-14): Combination of Precursor (B0pre-14) and Salt Exchange Compound (S-1-1)

¹H-NMR (DMSO, 400 MHz): δ(ppm)=1.24 (3H, s), 1.65 (2H, m), 1.84 (2H, m), 1.96 (2H, m), 4.60 (2H, t), 6.47 (1H, d), 7.35 (5H, m), 7.49 (6H, m), 7.77 (2H, m), 8.04 (2H, m), 8.23 (2H, m), 8.64 (2H, m)

### Preparation of Resist Composition

### Examples 1 to 16 and Comparative Examples 1 and 2

Each of the components shown in Table 1 was mixed and dissolved to prepare a resist composition of each Example.

TABLE 1

| | Component | Component | Component | Component |
|---|---|---|---|---|
| | (A) | (B) | (D) | (S) |
| Example 1 | (A)-1 | (B)-1 | (D)-1 | (S)-1 |
| | [100] | [26.2] | [5] | [8000] |
| Example 2 | (A)-1 | (B)-2 | (D)-1 | (S)-1 |
| | [100] | [25.8] | [5] | [8000] |
| Example 3 | (A)-1 | (B)-3 | (D)-1 | (S)-1 |
| | [100] | [25.8] | [5] | [8000] |
| Example 4 | (A)-1 | (B)-4 | (D)-1 | (S)-1 |
| | [100] | [26.2] | [5] | [8000] |
| Example 5 | (A)-1 | (B)-5 | (D)-1 | (S)-1 |
| | [100] | [26.6] | [5] | [8000] |
| Example 6 | (A)-1 | (B)-6 | (D)-1 | (S)-1 |
| | [100] | [26.2] | [5] | [8000] |
| Example 7 | (A)-1 | (B)-7 | (D)-1 | (S)-1 |
| | [100] | [25.8] | [5] | [8000] |
| Example 8 | (A)-1 | (B)-8 | (D)-1 | (S)-1 |
| | [100] | [29.8] | [5] | [8000] |
| Example 9 | (A)-1 | (B)-9 | (D)-1 | (S)-1 |
| | [100] | [28.1] | [5] | [8000] |
| Example 10 | (A)-1 | (B)-10 | (D)-1 | (S)-1 |
| | [100] | [31.4] | [5] | [8000] |
| Example 11 | (A)-1 | (B)-11 | (D)-1 | (S)-1 |
| | [100] | [32.6] | [5] | [8000] |
| Example 12 | (A)-1 | (B)-12 | (D)-1 | (S)-1 |
| | [100] | [32.6] | [5] | [8000] |
| Example 13 | (A)-1 | (B)-13 | (D)-1 | (S)-1 |
| | [100] | [22.5] | [5] | [8000] |
| Example 14 | (A)-1 | (B)-14 | (D)-1 | (S)-1 |
| | [100] | [24.0] | [5] | [8000] |
| Example 15 | (A)-2 | (B)-1 | (D)-1 | (S)-1 |
| | [100] | [28.1] | [5] | [8000] |
| Example 16 | (A)-3 | (B)-1 | (D)-1 | (S)-1 |
| | [100] | [28.1] | [5] | [8000] |
| Comparative | (A)-1 | (B)-15 | (D)-1 | (S)-1 |
| Example 1 | [100] | [23.9] | [5] | [8000] |
| Comparative | (A)-1 | (B)-16 | (D)-1 | (S)-1 |
| Example 2 | [100] | [24.4] | [5] | [8000] |

In Table 1, each abbreviation has the following meaning. The numerical values in the brackets are blending amounts (parts by mass).
(A)-1: polymer compound represented by Chemical Formula (A1-1) The weight-average molecular weight (Mw) of the polymer compound (A1-1) in terms of standard polystyrene determined by GPC measurement was 6900, and the polydispersity (Mw/Mn) thereof was 1.74. The copolymerization composition ratio (the ratio (molar ratio) of each constitutional unit in the structural formula) determined by ¹³C-NMR was l/m=60/40.
(A)-2: polymer compound represented by Chemical Formula (A1-2) The weight-average molecular weight (Mw) of the polymer compound (A1-2) in terms of standard polystyrene determined by GPC measurement was 7200, and the polydispersity (Mw/Mn) thereof was 1.72. The copolymerization composition ratio (the ratio (molar ratio) of each constitutional unit in the structural formula) determined by 13C-NMR was l/m=60/40.
(A)-3: polymer compound represented by Chemical Formula (A1-3) The weight-average molecular weight (Mw) of the polymer compound (A1-3) in terms of standard polystyrene determined by GPC measurement was 7100, and the polydispersity (Mw/Mn) thereof was 1.72. The copolymerization composition ratio (the ratio (molar ratio) of each constitutional unit in the structural formula) determined by ¹³C-NMR was l/m=60/40.

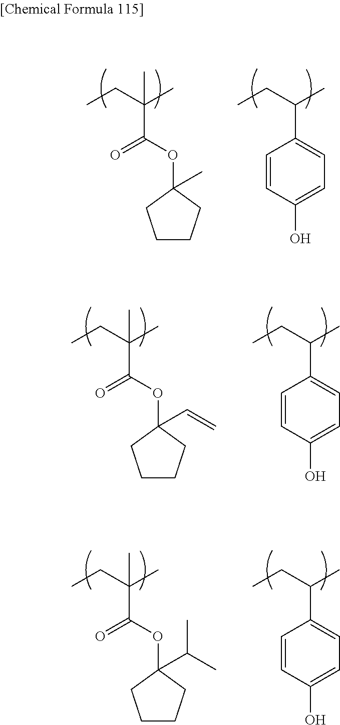

(B)-1 to (B)-14: compounds (B0-1) to (B0-14) described above
(B)-15: compound (B1-1) represented by Chemical Formula (B1-1)
(B)-16: compound (B1-2) represented by Chemical Formula (B1-2)
(D)-1: photodecomposable base formed of compound (D1-1) shown below

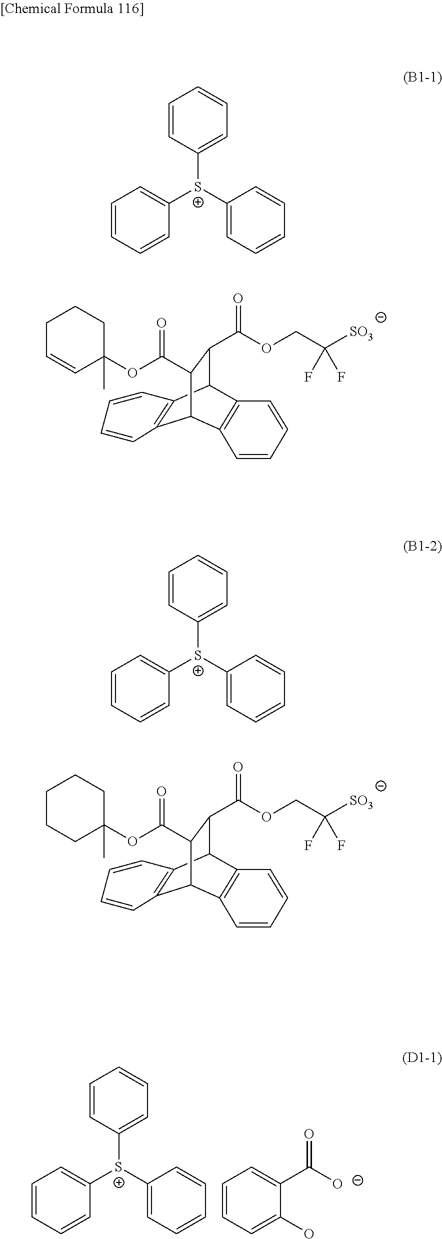

(S)-1: mixed solvent of propylene glycol monomethyl ether acetate/propylene glycol monomethyl ether=60/40 (mass ratio)

### <Resist Pattern Formation>

The resist composition of each Example was applied onto an 8-inch silicon substrate which had been subjected to a hexamethyldisilazane (HMDS) treatment using a spinner, the coated wafer was subjected to a pre-bake (PAB) treatment on a hot plate at a temperature of 110 °C for 60 seconds so that the coated wafer was dried to form a resist film having a film thickness of 50 nm.

Next, drawing (exposure) was carried out on the resist film by using an electron beam lithography apparatus JEOL-JBX-9300FS (manufactured by JEOL Ltd.), with the target size set to a 1:1 line and space pattern (hereinafter, referred to as “LS pattern”) of a line width of 50 nm, at an acceleration voltage of 100 kV. Thereafter, a post exposure bake (PEB) treatment was performed thereon at 100 °C for 60 seconds.

Subsequently, alkali development was performed at 23 °C for 60 seconds using a 2.38 mass % tetramethylammonium hydroxide (TMAH) aqueous solution “NMD-3” (trade name, manufactured by TOKYO OHKA KOGYO CO., LTD.). Thereafter, water rinsing was performed for 15 seconds using pure water.

As a result, a 1:1 LS pattern having a line width of 50 nm was formed.

### [Evaluation of Optimum Exposure Amount (Eop)]

An optimum exposure amount Bop (μC/cm²) at which an LS pattern with a target size was formed according to “Resist pattern formation” was determined. The results are listed in the columns of “Eop (μC/cm²)” in Table 2.

### [Evaluation of Reduction of Film Thickness (DL)]

The film thickness of the resist film immediately after the PAB treatment and the film thickness of the unexposed portion of the resist film after the water rinsing were measured in <Resist pattern formation> described above, and the amount of a decrease in the film thickness was measured. The results are listed in the columns of “DL (nm)” in Table 2.

TABLE 2

| | PAB | PEB | EOP | DL |
|---|---|---|---|---|
| | (°C) | (°C) | (μC/cm²) | (nm) |
| Example 1 | 110 | 100 | 80 | 4.0 |
| Example 2 | 110 | 100 | 82 | 4.6 |
| Example 3 | 110 | 100 | 83 | 4.3 |
| Example 4 | 110 | 100 | 83 | 4.0 |
| Example 5 | 110 | 100 | 84 | 3.9 |
| Example 6 | 110 | 100 | 85 | 4.0 |
| Example 7 | 110 | 100 | 87 | 4.3 |
| Example 8 | 110 | 100 | 82 | 4.0 |
| Example 9 | 110 | 100 | 84 | 4.0 |
| Example 10 | 110 | 100 | 84 | 4.2 |
| Example 11 | 110 | 100 | 77 | 3.9 |
| Example 12 | 110 | 100 | 78 | 3.9 |
| Example 13 | 110 | 100 | 84 | 4.2 |
| Example 14 | 110 | 100 | 86 | 4.3 |
| Example 15 | 110 | 100 | 79 | 3.9 |
| Example 16 | 110 | 100 | 80 | 4.0 |
| Comparative | 110 | 100 | 104 | 9.6 |
| Example 1 | | | | |
| Comparative | 110 | 100 | 113 | 9.9 |
| Example 2 | | | | |

As shown in the results listed in Table 2, it was confirmed that the resist compositions of Examples 1 to 16 achieved high sensitivity as compared with the resist compositions of Comparative Examples 1 and 2. In addition, it was also confirmed that a decrease in film thickness was further suppressed in the resist compositions of Examples 1 to 16 as compared with the resist compositions of Comparative Examples 1 and 2.

While preferred examples of the present invention have been described and illustrated above, it should be understood that these are exemplary of the present invention and are not to be considered as limiting. Additions, omissions, substitutions, and other modifications can be made without departing from the scope of the present invention. The present invention is not limited by the description above, but is limited only by the appended claims.

What is claimed is:

1. A resist composition which generates an acid upon light exposure and whose solubility in a developing solution is changed by an action of the acid, the resist composition comprising:
a base material component (A) whose solubility in a developing solution is changed by the action of the acid; and
a compound (B0) represented by General Formula (b0), 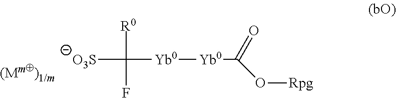
wherein in Formula (b0), M^{m+} represents an m-valent organic cation, m represents an integer of 1 or greater, R⁰ represents a fluorinated alkyl group having 1 to 5 carbon atoms or a fluorine atom, Vb⁰ represents an alkylene group, a fluorinated alkylene group, or a single bond, Yb⁰ represents a divalent linking group, Rog represents an acid dissociable group represented by General Formula (a0-pg), 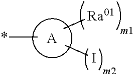
wherein in Formula (a0-pg), A represents a cyclic alkene having only one carbon-carbon unsaturated bond in a ring skeleton, Ra⁰¹ represents a monovalent hydrocarbon group having 1 to 20 carbon atoms, I represents an iodine atom, m1 represents an integer of 0 to 20, m2 represents an integer of 1 to 20, where m1+m2≤(a total number of carbon atoms constituting the ring skeleton of the cyclic alkene as A-1) is satisfied, in a case where m1 represents an integer of 2 or greater, a plurality of Ra⁰¹'s may be the same as or different from each other, and * represents a bonding site that is bonded to —O— (oxy group) in —Yb⁰—CO—O—.

2. The resist composition according to claim 1, wherein the compound (B0) is a compound represented by General Formula (b0-t1), 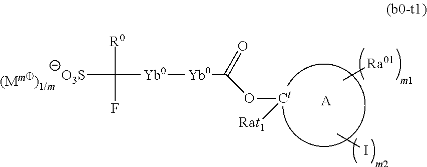
wherein in Formula (b0-t1), M^{m+} represents an m-valent organic cation, m represents an integer of 1 or greater, R⁰ represents a fluorinated alkyl group having 1 to 5 carbon atoms or a fluorine atom, Vb⁰ represents an alkylene group, a fluorinated alkylene group, or a single bond, Yb⁰ represents a divalent linking group, A represents a cyclic alkene having only one carbon-carbon unsaturated bond in a ring skeleton, C^{t} represents a tertiary carbon atom, Ra^{t1} represents a linear or branched alkyl group having 1 to 12 carbon atoms, Ra⁰¹ represents a monovalent hydrocarbon group having 1 to 20 carbon atoms, 1 represents an iodine atom, m1 represents an integer of 0 to 20, m2 represents an integer of 1 to 20, where m1+m2≤(the total number of carbon atoms constituting the ring skeleton of the cyclic alkene as A-1) is satisfied, and in a case where m1 represents an integer of 2 or greater, a plurality of Ra⁰¹'s may be the same as or different from each other.

3. The resist composition according to claim 1, wherein the compound (B0) is a compound represented by General Formula (b0-s1), 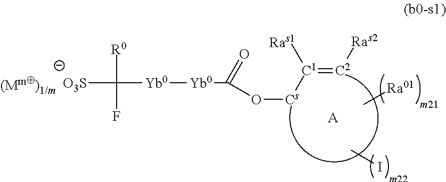
wherein in Formula (b0-s1), M^{m+} represents an m-valent organic cation, m represents an integer of 1 or greater, R⁰ represents a fluorinated alkyl group having 1 to 5 carbon atoms or a fluorine atom, Vb⁰ represents an alkylene group, a fluorinated alkylene group, or a single bond, Yb⁰ represents a divalent linking group, A represents a cyclic alkene having only one carbon-carbon unsaturated bond in a ring skeleton, C^{s} represents a secondary carbon atom, C¹ and C² each independently represent a carbon atom constituting the carbon-carbon unsaturated bond of the cyclic alkene as A, Ra^{s1} and Ra^{s2} each independently represent a hydrogen atom, an iodine atom, or a monovalent hydrocarbon group having 1 to 20 carbon atoms, Ra⁰¹ represents a monovalent hydrocarbon group having 1 to 20 carbon atoms, 1 represents an iodine atom, m21 represents an integer of 0 to 20, m22 represents an integer of 0 to 20, where m21+m22≤(the total number of carbon atoms constituting the ring skeleton of the cyclic alkene as A-3) is satisfied, at least one of Ra^{s1} or Ra^{s2} represents an iodine atom in a case where m22 represents 0, and a plurality of Ra⁰¹'s may be the same as or different from each other in a case where m21 represents an integer of 2 or greater.

4. The resist composition according to claim 1,
wherein, in General Formula (a0-pg), an iodine atom is bonded to at least one of carbon atoms constituting the carbon-carbon unsaturated bond in the ring skeleton of the cyclic alkene as A.

5. The resist composition according to claim 2, wherein the compound (B0) is a compound represented by General Formula (b0-t1-1), 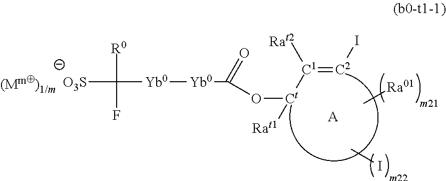
wherein in Formula (b0-t1-1), M^{m+} represents an m-valent organic cation, m represents an integer of 1 or greater, R⁰ represents a fluorinated alkyl group having 1 to 5 carbon atoms or a fluorine atom, Vb⁰ represents an alkylene group, a fluorinated alkylene group, or a single bond, Yb⁰ represents a divalent linking group, A represents a cyclic alkene having only one carbon-carbon unsaturated bond in a ring skeleton, C^{t} represents a tertiary carbon atom, C¹ and C² each independently represent a carbon atom constituting the carbon-carbon unsaturated bond of the cyclic alkene as A, Ra^{t1} represents a linear or branched alkyl group having 1 to 12 carbon atoms, Ra^{t2} represents a hydrogen atom or a monovalent hydrocarbon group having 1 to carbon atoms, Ra⁰¹ represents a monovalent hydrocarbon group having 1 to 20 carbon atoms, 1 represents an iodine atom, m11 represents an integer of 0 to 20, m12 represents an integer of 0 to 19, where m11+m12≤(the total number of carbon atoms constituting the ring skeleton of the cyclic alkene as A-3) is satisfied, and in a case where m11 represents an integer of 2 or greater, a plurality of Ra⁰¹'s may be the same as or different from each other.

6. The resist composition according to claim 3, wherein the compound (B0) is a compound represented by General Formula (b0-s1-1), 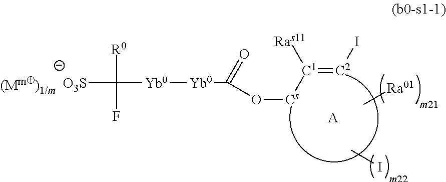
wherein in Formula (b0-s1-1), M^{m+} represents an m-valent organic cation, m represents an integer of 1 or greater, R⁰ represents a fluorinated alkyl group having 1 to 5 carbon atoms or a fluorine atom, Vb⁰ represents an alkylene group, a fluorinated alkylene group, or a single bond, Yb⁰ represents a divalent linking group, A represents a cyclic alkene having only one carbon-carbon unsaturated bond in a ring skeleton, C^{s} represents a secondary carbon atom, C¹ and C² each independently represent a carbon atom constituting the carbon-carbon unsaturated bond of the cyclic alkene as A, Ra^{s11} represents a hydrogen atom or a monovalent hydrocarbon group having 1 to 20 carbon atoms, Ra⁰¹ represents a monovalent hydrocarbon group having 1 to 20 carbon atoms, 1 represents an iodine atom, m21 represents an integer of 0 to 20, m22 represents an integer of 0 to 19, where m21+m22≤(the total number of carbon atoms constituting the ring skeleton of the cyclic alkene as A-3) is satisfied, and in a case where m21 represents an integer of 2 or greater, a plurality of Ra⁰¹'s may be the same as or different from each other.

7. The resist composition according to claim 3, wherein the compound (B0) is a compound represented by General Formula (b0-s1-2), 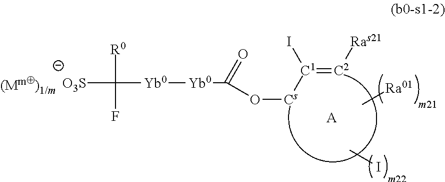
wherein in Formula (b0-s1-2), M^{m+} represents an m-valent organic cation, m represents an integer of 1 or greater, R⁰ represents a fluorinated alkyl group having 1 to 5 carbon atoms or a fluorine atom, Vb⁰ represents an alkylene group, a fluorinated alkylene group, or a single bond, Yb⁰ represents a divalent linking group, A represents a cyclic alkene having only one carbon-carbon unsaturated bond in a ring skeleton, C^{s} represents a secondary carbon atom, C¹ and C² each independently represent a carbon atom constituting the carbon-carbon unsaturated bond of the cyclic alkene as A, Ra^{s21} represents a hydrogen atom or a monovalent hydrocarbon group having 1 to 20 carbon atoms, Ra⁰¹ represents a monovalent hydrocarbon group having 1 to 20 carbon atoms, 1 represents an iodine atom, m21 represents an integer of 0 to 20, m22 represents an integer of 0 to 19, where m21+m22≤(the total number of carbon atoms constituting the ring skeleton of the cyclic alkene as A-3) is satisfied, and in a case where m21 represents an integer of 2 or greater, a plurality of Ra⁰¹'s may be the same as or different from each other.

8. The resist composition according to claim 3, wherein the compound (B0) is a compound represented by General Formula (b0-s1-3), 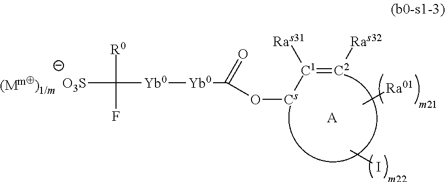
wherein in Formula (b0-s1-3), M^{m+} represents an m-valent organic cation, m represents an integer of 1 or greater, R⁰ represents a fluorinated alkyl group having 1 to 5 carbon atoms or a fluorine atom, Vb⁰ represents an alkylene group, a fluorinated alkylene group, or a single bond, Yb⁰ represents a divalent linking group, A represents a cyclic alkene having only one carbon-carbon unsaturated bond in a ring skeleton, C^{s} represents a secondary carbon atom, C¹ and C² each independently represent a carbon atom constituting the carbon-carbon unsaturated bond of the cyclic alkene as A, Ra^{s31} and Ra^{s32} each independently represent a hydrogen atom or a monovalent hydrocarbon group having 1 to 20 carbon atoms, Ra⁰¹ represents a monovalent hydrocarbon group having 1 to 20 carbon atoms, m31 represents an integer of 0 to 20, m32 represents an integer of 1 to 20, where m31+m32≤(the total number of carbon atoms constituting the ring skeleton of the cyclic alkene as A-3) is satisfied, and in a case where m31 represents an integer of 2 or greater, a plurality of Ra⁰¹'s may be the same as or different from each other.

9. The resist composition according to claim 1, further comprising:
a photodecomposable base that controls diffusion of the acid generated upon light exposure, is decomposed upon light exposure, and loses acid diffusion controllability.

10. A resist pattern formation method comprising:
forming a resist film on a support using the resist composition according to claim 1;
exposing the resist film to light; and
developing the resist film exposed to light to form a resist pattern.

11. The resist pattern formation method according to claim 10, wherein the resist film is exposed to an extreme ultraviolet ray or an electron beam in.

12. A compound which is represented by General Formula (b0), 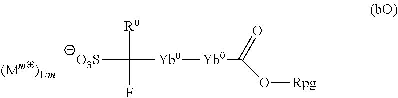
wherein in Formula (b0), M^{m+} represents an m-valent organic cation, m represents an integer of 1 or greater, R⁰ represents a fluorinated alkyl group having 1 to 5 carbon atoms or a fluorine atom, Vb⁰ represents an alkylene group, a fluorinated alkylene group, or a single bond, Yb⁰ represents a divalent linking group, Rpg represents an acid dissociable group represented by General Formula (a0-pg), 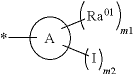
wherein in Formula (a0-pg), A represents a cyclic alkene having only one carbon-carbon unsaturated bond in a ring skeleton, Ra⁰¹ represents a monovalent hydrocarbon group having 1 to 20 carbon atoms, 1 represents an iodine atom, m1 represents an integer of 0 to 20, m2 represents an integer of 1 to 20, where m1+m2≤(a total number of carbon atoms constituting the ring skeleton of the cyclic alkene as A-1) is satisfied, in a case where m1 represents an integer of 2 or greater, a plurality of Ra⁰¹'s may be the same as or different from each other, and * represents a bonding site that is bonded to —O— (oxy group) in —Yb⁰—CO—O—.

13. The compound according to claim 12, wherein the compound is a compound represented by General Formula (b0-t1), 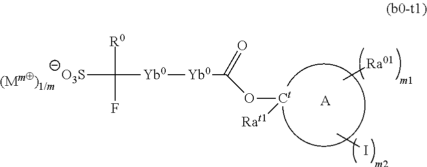
wherein in Formula (b0-t1), M^{m+} represents an m-valent organic cation, m represents an integer of 1 or greater, R⁰ represents a fluorinated alkyl group having 1 to 5 carbon atoms or a fluorine atom, Vb⁰ represents an alkylene group, a fluorinated alkylene group, or a single bond, Yb⁰ represents a divalent linking group, A represents a cyclic alkene having only one carbon-carbon unsaturated bond in a ring skeleton, C^{t} represents a tertiary carbon atom, Ra^{t1} represents a linear or branched alkyl group having 1 to 12 carbon atoms, Ra⁰¹ represents a monovalent hydrocarbon group having 1 to 20 carbon atoms, 1 represents an iodine atom, m1 represents an integer of 0 to 20, m2 represents an integer of 1 to 20, where m1+m2≤(the total number of carbon atoms constituting the ring skeleton of the cyclic alkene as A-1) is satisfied, and in a case where m1 represents an integer of 2 or greater, a plurality of Ra⁰¹'s may be the same as or different from each other.

14. The compound according to claim 12, wherein the compound (B0) is a compound represented by General Formula (b0-s1), 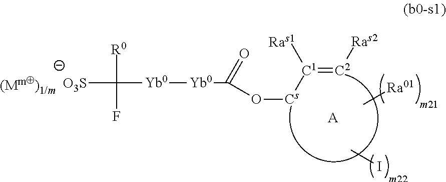
wherein in Formula (b0-s1), M^{m+} represents an m-valent organic cation, m represents an integer of 1 or greater, R⁰ represents a fluorinated alkyl group having 1 to 5 carbon atoms or a fluorine atom, Vb⁰ represents an alkylene group, a fluorinated alkylene group, or a single bond, Yb⁰ represents a divalent linking group, A represents a cyclic alkene having only one carbon-carbon unsaturated bond in a ring skeleton, C^{s} represents a secondary carbon atom, C¹ and C² each independently represent a carbon atom constituting the carbon-carbon unsaturated bond of the cyclic alkene as A, Ra^{s1} and Ra^{s2} each independently represent a hydrogen atom, an iodine atom, or a monovalent hydrocarbon group having 1 to 20 carbon atoms, Ra⁰¹ represents a monovalent hydrocarbon group having 1 to 20 carbon atoms, 1 represents an iodine atom, m21 represents an integer of 0 to 20, m22 represents an integer of 0 to 20, where m21+m22≤(the total number of carbon atoms constituting the ring skeleton of the cyclic alkene as A-3) is satisfied, at least one of Ra^{s1} or Ra^{s2} represents an iodine atom in a case where m22 represents 0, and a plurality of Ra⁰¹'s may be the same as or different from each other in a case where m21 represents an integer of 2 or greater.

15. The compound according to claim 12,
wherein, in General Formula (a0-pg), an iodine atom is bonded to at least one of carbon atoms constituting the carbon-carbon unsaturated bond in the ring skeleton of the cyclic alkene as A.

16. The compound according to claim 13,
wherein the compound is a compound represented by General Formula (b0-t1-1), 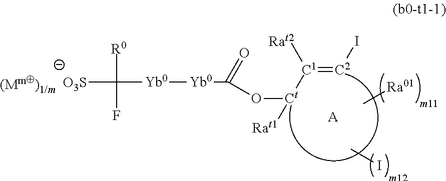
wherein in Formula (b0-t1-1), M^{m+} represents an m-valent organic cation, m represents an integer of 1 or greater, R⁰ represents a fluorinated alkyl group having 1 to 5 carbon atoms or a fluorine atom, Vb⁰ represents an alkylene group, a fluorinated alkylene group, or a single bond, Yb⁰ represents a divalent linking group, A represents a cyclic alkene having only one carbon-carbon unsaturated bond in a ring skeleton, C^{t} represents a tertiary carbon atom, C¹ and C² each independently represent a carbon atom constituting the carbon-carbon unsaturated bond of the cyclic alkene as A, Ra^{t1} represents a linear or branched alkyl group having 1 to 12 carbon atoms, Ra^{t2} represents a hydrogen atom or a monovalent hydrocarbon group having 1 to carbon atoms, Ra⁰¹ represents a monovalent hydrocarbon group having 1 to 20 carbon atoms, 1 represents an iodine atom, m11 represents an integer of 0 to 20, m12 represents an integer of 0 to 19, where m11+m12≤(the total number of carbon atoms constituting the ring skeleton of the cyclic alkene as A-3) is satisfied, and in a case where m11 represents an integer of 2 or greater, a plurality of Ra⁰¹'s may be the same as or different from each other.

17. The compound according to claim 14, wherein the compound is a compound represented by General Formula (b0-s1-1), 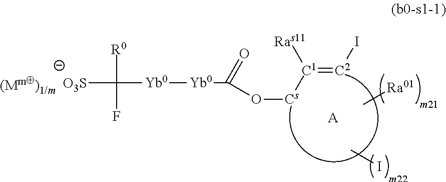
wherein in Formula (b0-s1-1), M^{m+} represents an m-valent organic cation, m represents an integer of 1 or greater, R⁰ represents a fluorinated alkyl group having 1 to 5 carbon atoms or a fluorine atom, Vb⁰ represents an alkylene group, a fluorinated alkylene group, or a single bond, Yb⁰ represents a divalent linking group, A represents a cyclic alkene having only one carbon-carbon unsaturated bond in a ring skeleton, C^{s} represents a secondary carbon atom, C¹ and C² each independently represent a carbon atom constituting the carbon-carbon unsaturated bond of the cyclic alkene as A, Ra^{s11} represents a hydrogen atom or a monovalent hydrocarbon group having 1 to 20 carbon atoms, Ra⁰¹ represents a monovalent hydrocarbon group having 1 to 20 carbon atoms, 1 represents an iodine atom, m21 represents an integer of 0 to 20, m22 represents an integer of 0 to 19, where m21+m22≤(the total number of carbon atoms constituting the ring skeleton of the cyclic alkene as A-3) is satisfied, and in a case where m21 represents an integer of 2 or greater, a plurality of Ra⁰¹'s may be the same as or different from each other.

18. The compound according to claim 14, wherein the compound is a compound represented by General Formula (b0-s1-2), 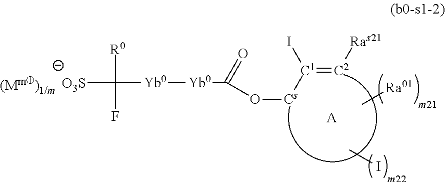
wherein in Formula (b0-s1-2), M^{m+} represents an m-valent organic cation, m represents an integer of 1 or greater, R⁰ represents a fluorinated alkyl group having 1 to 5 carbon atoms or a fluorine atom, Vb⁰ represents an alkylene group, a fluorinated alkylene group, or a single bond, Yb⁰ represents a divalent linking group, A represents a cyclic alkene having only one carbon-carbon unsaturated bond in a ring skeleton, C^{s} represents a secondary carbon atom, C¹ and C² each independently represent a carbon atom constituting the carbon-carbon unsaturated bond of the cyclic alkene as A, Ra^{s21} represents a hydrogen atom or a monovalent hydrocarbon group having 1 to 20 carbon atoms, Ra⁰¹ represents a monovalent hydrocarbon group having 1 to 20 carbon atoms, 1 represents an iodine atom, m21 represents an integer of 0 to 20, m22 represents an integer of 0 to 19, where m21+m22≤(the total number of carbon atoms constituting the ring skeleton of the cyclic alkene as A-3) is satisfied, and in a case where m21 represents an integer of 2 or greater, a plurality of Ra⁰¹'s may be the same as or different from each other.

19. The compound according to claim 14, wherein the compound is a compound represented by General Formula (b0-s1-3), 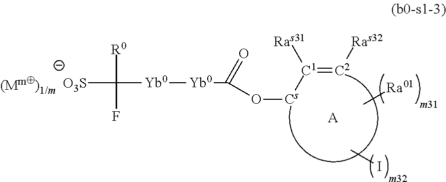
wherein in Formula (b0-s1-3), M^{m+} represents an m-valent organic cation, m represents an integer of 1 or greater, R⁰ represents a fluorinated alkyl group having 1 to 5 carbon atoms or a fluorine atom, Vb⁰ represents an alkylene group, a fluorinated alkylene group, or a single bond, Yb⁰ represents a divalent linking group, A represents a cyclic alkene having only one carbon-carbon unsaturated bond in a ring skeleton, C^{s} represents a secondary carbon atom, C¹ and C² each independently represent a carbon atom constituting the carbon-carbon unsaturated bond of the cyclic alkene as A, Ra^{s31} and Ra^{s32} each independently represent a hydrogen atom or a monovalent hydrocarbon group having 1 to 20 carbon atoms, Ra⁰¹ represents a monovalent hydrocarbon group having 1 to 20 carbon atoms, m31 represents an integer of 0 to 20, m32 represents an integer of 1 to 20, where m31+m32≤(the total number of carbon atoms constituting the ring skeleton of the cyclic alkene as A-3) is satisfied, and in a case where m31 represents an integer of 2 or greater, a plurality of Ra⁰¹'s may be the same as or different from each other.

20. A compound which is represented by General Formula (b0-p), 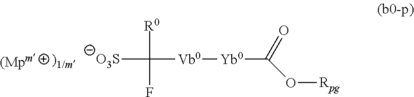
wherein in Formula (b0-p), Mp^{m′+} represents an organic cation or a metal cation, m′ represents an integer of 1 or greater, R⁰ represents a fluorinated alkyl group having 1 to 5 carbon atoms or a fluorine atom, Vb⁰ represents an alkylene group, a fluorinated alkylene group, or a single bond, Yb⁰ represents a divalent linking group, and Rpg represents an acid dissociable group represented by General Formula (a0-pg), 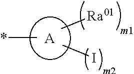
wherein in Formula (a0-pg), A represents a cyclic alkene having only one carbon-carbon unsaturated bond in a ring skeleton, Ra⁰¹ represents a monovalent hydrocarbon group having 1 to 20 carbon atoms, 1 represents an iodine atom, m1 represents an integer of 0 to 20, m2 represents an integer of 1 to 20, where m1+m2≤(a total number of carbon atoms constituting the ring skeleton of the cyclic alkene as A-1) is satisfied, in a case where m1 represents an integer of 2 or greater, a plurality of Ra⁰¹'s may be the same as or different from each other, and * represents a bonding site that is bonded to —O— (oxy group) in —Yb⁰—CO—O—.
